SEQUENCE LISTING (294 sequences)

SEQ ID NO: 1 - protein, 8 aa, Artificial Sequence
  Description of Artificial Sequence Synthetic peptide sequence motif
XXXXXXXX

SEQ ID NO: 2 - protein, 9 aa, Artificial Sequence
  Description of Artificial Sequence Synthetic peptide sequence motif
XXXXXXXXX

SEQ ID NO: 3 - protein, 10 aa, Artificial Sequence
  Description of Artificial Sequence Synthetic peptide sequence motif
XXXXXXXXXX

SEQ ID NO: 4 - protein, 11 aa, Artificial Sequence
  Description of Artificial Sequence Synthetic peptide sequence motif
XXXXXXXXXXX

SEQ ID NO: 5 - protein, 12 aa, Artificial Sequence
  Description of Artificial Sequence Synthetic peptide sequence motif
XXXXXXXXXXXX

SEQ ID NO: 6 - protein, 13 aa, Artificial Sequence
  Description of Artificial Sequence Synthetic peptide sequence motif
XXXXXXXXXXXXX

SEQ ID NO: 7 - protein, 8 aa, Artificial Sequence
  Description of Artificial Sequence Synthetic peptide sequence motif
XXXXXXXX

SEQ ID NO: 8 - protein, 9 aa, Artificial Sequence
  Description of Artificial Sequence Synthetic peptide sequence motif
XXXXXXXXX

SEQ ID NO: 9 - protein, 10 aa, Artificial Sequence
  Description of Artificial Sequence Synthetic peptide sequence motif
XXXXXXXXXX

SEQ ID NO: 10 - protein, 11 aa, Artificial Sequence
  Description of Artificial Sequence Synthetic peptide sequence motif
XXXXXXXXXXX

SEQ ID NO: 11 - protein, 12 aa, Artificial Sequence
  Description of Artificial Sequence Synthetic peptide sequence motif
XXXXXXXXXXXX

SEQ ID NO: 12 - protein, 7 aa, Artificial Sequence
  Description of Artificial Sequence Synthetic peptide sequence motif
XXXXXXX

SEQ ID NO: 13 - protein, 8 aa, Artificial Sequence
  Description of Artificial Sequence Synthetic peptide sequence motif
XXXXXXXX

SEQ ID NO: 14 - protein, 9 aa, Artificial Sequence
  Description of Artificial Sequence Synthetic peptide sequence motif
XXXXXXXXX

SEQ ID NO: 15 - protein, 10 aa, Artificial Sequence
  Description of Artificial Sequence Synthetic peptide sequence motif
XXXXXXXXXX

SEQ ID NO: 16 - protein, 11 aa, Artificial Sequence
  Description of Artificial Sequence Synthetic peptide sequence motif
XXXXXXXXXXX

SEQ ID NO: 17 - protein, 10 aa, Artificial Sequence
  Description of Artificial Sequence Synthetic peptide sequence motif
XXXXXXXXXX

SEQ ID NO: 18 - protein, 9 aa, Artificial Sequence
  Description of Artificial Sequence Synthetic peptide sequence motif
XXXXXXXXX

SEQ ID NO: 19 - protein, 74 aa, Homo sapiens [sequence omitted]

SEQ ID NO: 20 - protein, 129 aa, Homo sapiens
MEGLVFSRLSPEYYDPARAHLRDGEKSCPCGQEGPQGDLLTKTQELGRDYRTCLTIVQKLKKMVDKPTQRSVSNAATRVCRTGRSRWRDVCRNFMRRYQSRVIQGLVAGETAQQICEDLRLCIPSTGPL

SEQ ID NO: 21 - protein, 145 aa, Homo sapiens
MATWALLLLAAMLLGNPGLVFSRLSPEYYDLARAHLRDEEKSCPCLAQEGPQGDLLTKTQELGRDYRTCLTIVQKLKKMVDKPTQRSVSNAATRVCRTGRSRWRDVCRNFMRRYQSRVTQGLVAGETAQQICEDLRLCIPSTGPL

SEQ ID NO: 22 - protein, 172 aa, Homo sapiens
MATWALLLLAAMLLGNPGLEVSVSPKGKNTSGRESGFGWAIWMEGLVFSRLSPEYYDLARAHLRDEEKSCPCLAQEGPQGDLLTKTQELGRDYRTCLTIVQKLKKMVDKPTQRSVSNAATRVCRTGRSRWRDVCRNFMRRYQSRVIQGLVAGETAQQICEDLRLCIPSTGPL

SEQ ID NO: 23 - protein, 22 aa, Artificial Sequence
  Description of Artificial Sequence Synthetic peptide moiety
TRVSRTGRSRWRDWSRNFMRAA

SEQ ID NO: 24 - protein, 30 aa, Artificial Sequence
  Description of Artificial Sequence Synthetic peptide moiety
TRVSRTGRSRWRDWSRNFMRAARRRRRRRR

SEQ ID NO: 25 - protein, 30 aa, Artificial Sequence
  Description of Artificial Sequence Synthetic peptide moiety
TRVSRTGRSRWRDWSRNFMRAAXXXXXXXX

SEQ ID NO: 26 - protein, 30 aa, Artificial Sequence
  Description of Artificial Sequence Synthetic peptide moiety
TRVSRTGRSRWRDWSRNFMRAAKKKKKKKK

SEQ ID NO: 27 - protein, 26 aa, Artificial Sequence
  Description of Artificial Sequence Synthetic peptide moiety
TRVSRTGRSRWRDWSRNFMRAAKKKK

SEQ ID NO: 28 - protein, 27 aa, Artificial Sequence
  Description of Artificial Sequence Synthetic peptide moiety
TRVSRTGRSRWRDWSRNFMRAAKKKKK

SEQ ID NO: 29 - protein, 28 aa, Artificial Sequence
  Description of Artificial Sequence Synthetic peptide moiety
TRVSRTGRSRWRDWSRNFMRAAKKKKKK

SEQ ID NO: 30 - protein, 32 aa, Artificial Sequence
  Description of Artificial Sequence Synthetic peptide moiety
KKKKAATRVSRTGRSRWRDWSRNFMRAAKKKK

SEQ ID NO: 31 - protein, 26 aa, Artificial Sequence
  Description of Artificial Sequence Synthetic peptide moiety
TRVSRTGRSRWRDWSRNFMRAARRRR

SEQ ID NO: 32 - protein, 27 aa, Artificial Sequence
  Description of Artificial Sequence Synthetic peptide moiety
TRVSRTGRSRWRDWSRNFMRAARRRRR

SEQ ID NO: 33 - protein, 28 aa, Artificial Sequence
  Description of Artificial Sequence Synthetic peptide moiety
TRVSRTGRSRWRDWSRNFMRAARRRRRR

SEQ ID NO: 34 - protein, 32 aa, Artificial Sequence
  Description of Artificial Sequence Synthetic peptide moiety
RRRRAATRVSRTGRSRWRDWSRNFMRAARRRR

SEQ ID NO: 35 - protein, 22 aa, Artificial Sequence
  Description of Artificial Sequence Synthetic peptide moiety
TRVSRTGRSRWRDWSRNFMRRR

SEQ ID NO: 36 - protein, 22 aa, Artificial Sequence
  Description of Artificial Sequence Synthetic peptide moiety
TRVSRTGRSRWRDWSRNWMRRR

SEQ ID NO: 37 - protein, 22 aa, Artificial Sequence
  Description of Artificial Sequence Synthetic peptide moiety
ERVSRTGRSRWRDWSRNFMRRR

SEQ ID NO: 38 - protein, 22 aa, Artificial Sequence
  Description of Artificial Sequence Synthetic peptide moiety
RRVSRTGRSRWRDWSRNFMRRR

SEQ ID NO: 39 - protein, 22 aa, Artificial Sequence
  Description of Artificial Sequence Synthetic peptide moiety
AATRVSRTGRSRWRDWSRNFMR

SEQ ID NO: 40 - protein, 23 aa, Artificial Sequence
  Description of Artificial Sequence Synthetic peptide moiety
XTRVSRTGRSRWRDWSRNFMRAA

SEQ ID NO: 41 - protein, 22 aa, Artificial Sequence
  Description of Artificial Sequence Synthetic peptide moiety
TRVSRTGRSRWRDWSRNFMRLL

SEQ ID NO: 42 - protein, 20 aa, Artificial Sequence
  Description of Artificial Sequence Synthetic peptide moiety
TRVSRTGRSRWRDVSRNFMR

SEQ ID NO: 43 - protein, 47 aa, Artificial Sequence
  Description of Artificial Sequence Synthetic peptide moiety
TRVSRTGRSRWRDWSRNFMRAAXTRVSRTGRSRWRDWSRNFMRAAXK

SEQ ID NO: 44 - protein, 45 aa, Artificial Sequence
  Description of Artificial Sequence Synthetic peptide moiety
TRVSRTGRSRWRDWSRNFMRAAXAATRVSRTGRSRWRDWSRNFMR

SEQ ID NO: 45 - protein, 20 aa, Artificial Sequence
  Description of Artificial Sequence Synthetic peptide moiety
TRVXRTGRSRWRDWDRNFMR

SEQ ID NO: 46 - protein, 20 aa, Artificial Sequence
  Description of Artificial Sequence Synthetic peptide moiety
TRVDRTGRSRWRDWXRNFMR

SEQ ID NO: 47 - protein, 20 aa, Artificial Sequence
  Description of Artificial Sequence Synthetic peptide moiety
TRVXRTGRSRWRDVDRNFMR

SEQ ID NO: 48 - protein, 20 aa, Artificial Sequence
  Description of Artificial Sequence Synthetic peptide moiety
TRVDRTGRSRWRDVXRNFMR

SEQ ID NO: 49 - protein, 27 aa, Artificial Sequence
  Description of Artificial Sequence Synthetic peptide moiety
RSVSNAATRVSRTGRSRWRDWSRNFMR

SEQ ID NO: 50 - protein, 33 aa, Artificial Sequence
  Description of Artificial Sequence Synthetic peptide moiety
RDYRTSLTIVQKXTRVSRTGRSRWRDWSRNFMR

SEQ ID NO: 51 - protein, 22 aa, Artificial Sequence
  Description of Artificial Sequence Synthetic peptide moiety
TRVSRTGRSRWRDWSRNFMRLL

SEQ ID NO: 52 - protein, 22 aa, Artificial Sequence
  Description of Artificial Sequence Synthetic peptide moiety
TRVSRTGRSRWRDWSRNFMRAA

SEQ ID NO: 53 - protein, 30 aa, Artificial Sequence; identical to SEQ ID NO: 26
  Description of Artificial Sequence Synthetic peptide moiety

SEQ ID NO: 54 - protein, 30 aa, Artificial Sequence; identical to SEQ ID NO: 25
  Description of Artificial Sequence Synthetic peptide moiety

SEQ ID NO: 55 - protein, 20 aa, Artificial Sequence
  Description of Artificial Sequence Synthetic peptide moiety
TRVSRTGRSRWRDWSRNFMR

SEQ ID NO: 56 - protein, 28 aa, Artificial Sequence
  Description of Artificial Sequence Synthetic peptide moiety
TRVSRTGRSRWRDWSRNFMRKKKKKKKK

SEQ ID NO: 57 - protein, 28 aa, Artificial Sequence
  Description of Artificial Sequence Synthetic peptide moiety
TRVSRTGRSRWRDWSRNFMRXXXXXXXX

SEQ ID NO: 58 - protein, 30 aa, Artificial Sequence
  Description of Artificial Sequence Synthetic peptide moiety
KKKKKKKKAATRVSRTGRSRWRDWSRNFMR

SEQ ID NO: 59 - protein, 20 aa, Artificial Sequence
  Description of Artificial Sequence Synthetic peptide moiety
TRVARTGRSRWRDWARNFMR

SEQ ID NO: 60 - protein, 20 aa, Artificial Sequence
  Description of Artificial Sequence Synthetic peptide moiety
TRVARTGRSRWRDVARNFMR

SEQ ID NO: 61 - protein, 22 aa, Artificial Sequence
  Description of Artificial Sequence Synthetic peptide moiety
TRVARTGRSRWRDVARNFMRAA

SEQ ID NO: 62 - protein, 30 aa, Artificial Sequence
  Description of Artificial Sequence Synthetic peptide moiety
TRVARTGRSRWRDVARNFMRAARRRRRRRR

SEQ ID NO: 63 - protein, 30 aa, Artificial Sequence
  Description of Artificial Sequence Synthetic peptide moiety
TRVARTGRSRWRDVARNFMRAAXXXXXXXX

SEQ ID NO: 64 - protein, 30 aa, Artificial Sequence
  Description of Artificial Sequence Synthetic peptide moiety
TRVARTGRSRWRDVARNFMRAAKKKKKKKK

SEQ ID NO: 65 - protein, 22 aa, Artificial Sequence
  Description of Artificial Sequence Synthetic peptide moiety
TRVARTGRSRWRDVARNFMRRR

SEQ ID NO: 66 - protein, 22 aa, Artificial Sequence
  Description of Artificial Sequence Synthetic peptide moiety
TRVARTGRSRWRDVARNWMRRR

SEQ ID NO: 67 - protein, 22 aa, Artificial Sequence
  Description of Artificial Sequence Synthetic peptide moiety
ERVARTGRSRWRDVARNFMRRR

SEQ ID NO: 68 - protein, 23 aa, Artificial Sequence
  Description of Artificial Sequence Synthetic peptide moiety
AATRVARTGRSRWRDVARNFMRR

SEQ ID NO: 69 - protein, 28 aa, Artificial Sequence
  Description of Artificial Sequence Synthetic peptide moiety
RSVSNAATRVARTGRSRWRDVARNFMRR

SEQ ID NO: 70 - protein, 32 aa, Artificial Sequence
  Description of Artificial Sequence Synthetic peptide moiety
KKKKAATRVARTGRSRWRDVARNFMRAAKKKK

SEQ ID NO: 71 - protein, 32 aa, Artificial Sequence
  Description of Artificial Sequence Synthetic peptide moiety
RRRRAATRVARTGRSRWRDVARNFMRAARRRR

SEQ ID NO: 72 - protein, 23 aa, Artificial Sequence
  Description of Artificial Sequence Synthetic peptide moiety
XTRVARTGRSRWRDVARNFMRAA

SEQ ID NO: 73 - protein, 22 aa, Artificial Sequence
  Description of Artificial Sequence Synthetic peptide moiety
TRVARTGRSRWRDVARNFMRLL

SEQ ID NO: 74 - protein, 22 aa, Artificial Sequence
  Description of Artificial Sequence Synthetic peptide moiety
TRVARTGRSRWRDVARNFMRAA

SEQ ID NO: 75 - protein, 30 aa, Artificial Sequence; identical to SEQ ID NO: 64
  Description of Artificial Sequence Synthetic peptide moiety

SEQ ID NO: 76 - protein, 30 aa, Artificial Sequence; identical to SEQ ID NO: 63
  Description of Artificial Sequence Synthetic peptide moiety

SEQ ID NO: 77 - protein, 30 aa, Artificial Sequence
  Description of Artificial Sequence Synthetic peptide moiety
KKKKKKKKAATRVARTGRSRWRDVARNFMR

SEQ ID NO: 78 - protein, 20 aa, Artificial Sequence
  Description of Artificial Sequence Synthetic peptide moiety
TRVARTGRSRWRDWARNFMR

SEQ ID NO: 79 - protein, 20 aa, Artificial Sequence
  Description of Artificial Sequence Synthetic peptide moiety
TRVARTGRSRWRDVARNFMR

SEQ ID NO: 80 - protein, 28 aa, Artificial Sequence
  Description of Artificial Sequence Synthetic peptide moiety
TRVARTGRSRWRDVARNFMRKKKKKKKK

SEQ ID NO: 81 - protein, 28 aa, Artificial Sequence
  Description of Artificial Sequence Synthetic peptide moiety
TRVARTGRSRWRDVARNFMRXXXXXXXX

SEQ ID NO: 82 - protein, 20 aa, Artificial Sequence
  Description of Artificial Sequence Synthetic peptide moiety
TRVARTGRSRWRDVARNFAR

SEQ ID NO: 83 - protein, 20 aa, Artificial Sequence
  Description of Artificial Sequence Synthetic peptide moiety
TRVSRTGRSRWRDWSRNFAR

SEQ ID NO: 84 - protein, 21 aa, Artificial Sequence
  Description of Artificial Sequence Synthetic peptide moiety
TRVSRTGRSRWRDWSRNFMRC

SEQ ID NO: 85 - protein, 20 aa, Artificial Sequence
  Description of Artificial Sequence Synthetic peptide moiety
TRVSRTGRSRWRDWSRNFMR

SEQ ID NO: 86 - protein, 21 aa, Artificial Sequence
  Description of Artificial Sequence Synthetic peptide moiety
TRVSRTGRSRWRDWSRNFMRK

SEQ ID NO: 87 - protein, 21 aa, Artificial Sequence
  Description of Artificial Sequence Synthetic peptide moiety
TRVSRTGRSRWRDWSRNFMRK

SEQ ID NO: 88 - protein, 21 aa, Artificial Sequence
  Description of Artificial Sequence Synthetic peptide moiety
TRVSRTGRSRWRDWSRNFMRK

SEQ ID NO: 89 - protein, 20 aa, Artificial Sequence
  Description of Artificial Sequence Synthetic peptide moiety
TRVSRTGRSRWRDWSRNFMR

SEQ ID NO: 90 - protein, 22 aa, Artificial Sequence
  Description of Artificial Sequence Synthetic peptide moiety
TRVSRTGRSRWRDWSRNFMRLL

SEQ ID NO: 91 - protein, 20 aa, Artificial Sequence
  Description of Artificial Sequence Synthetic peptide moiety
TRVARTGRSRWRDVARNFMR

SEQ ID NO: 92 - protein, 20 aa, Artificial Sequence
  Description of Artificial Sequence Synthetic peptide moiety
TRVARTGRSRWRDWARNFMR

SEQ ID NO: 93 - protein, 21 aa, Artificial Sequence
  Description of Artificial Sequence Synthetic peptide moiety
TRVARTGRSRWRDVARNFMRK

SEQ ID NO: 94 - protein, 21 aa, Artificial Sequence
  Description of Artificial Sequence Synthetic peptide moiety
TRVARTGRSRWRDVARNFMRK

SEQ ID NO: 95 - protein, 21 aa, Artificial Sequence
  Description of Artificial Sequence Synthetic peptide moiety
TRVARTGRSRWRDVARNFMRK

SEQ ID NO: 96 - protein, 22 aa, Artificial Sequence
  Description of Artificial Sequence Synthetic peptide moiety
TRVARTGRSRWRDVARNFMRLL

SEQ ID NO: 97 - protein, 20 aa, Artificial Sequence
  Description of Artificial Sequence Synthetic peptide moiety
TRVARTGRSRWRDVARNFMR

SEQ ID NO: 98 - protein, 20 aa, Artificial Sequence
  Description of Artificial Sequence Synthetic peptide moiety
TRVSRTGRSRWRDWSRNFMR

SEQ ID NO: 99 - protein, 20 aa, Artificial Sequence
  Description of Artificial Sequence Synthetic peptide moiety
TRVARTGRSRWRDWARNFMR

SEQ ID NO: 100 - protein, 20 aa, Artificial Sequence
  Description of Artificial Sequence Synthetic peptide moiety
TRVARTGRSRWRDVARNFMR

SEQ ID NO: 101 - protein, 21 aa, Artificial Sequence
  Description of Artificial Sequence Synthetic peptide moiety
TRVSRTGRSRWRDWSRNFMRC

SEQ ID NO: 102 - protein, 23 aa, Artificial Sequence
  Description of Artificial Sequence Synthetic peptide moiety
TRVSRTGRSRWRDWSRNFMRAAK

SEQ ID NO: 103 - protein, 23 aa, Artificial Sequence
  Description of Artificial Sequence Synthetic peptide moiety
TRVSRTGRSRWRDWSRNFMRAAK

SEQ ID NO: 104 - protein, 23 aa, Artificial Sequence
  Description of Artificial Sequence Synthetic peptide moiety
TRVARTGRSRWRDVARNFMRAAK

SEQ ID NO: 105 - protein, 23 aa, Artificial Sequence
  Description of Artificial Sequence Synthetic peptide moiety
TRVARTGRSRWRDVARNFMRAAK

SEQ ID NO: 106 - protein, 23 aa, Artificial Sequence
  Description of Artificial Sequence Synthetic peptide moiety
TRVSRTGRSRWRDWSRNFMRAAK

SEQ ID NO: 107 - protein, 23 aa, Artificial Sequence
  Description of Artificial Sequence Synthetic peptide moiety
TRVSRTGRSRWRDWSRNFMRAAK

SEQ ID NO: 108 - protein, 23 aa, Artificial Sequence
  Description of Artificial Sequence Synthetic peptide moiety
TRVSRTGRSRWRDWSRNFMRAAK

SEQ ID NO: 109 - protein, 13 aa, Artificial Sequence
  Description of Artificial Sequence Synthetic peptide moiety
RSRWRDWSRNFMR

SEQ ID NO: 110 - protein, 13 aa, Artificial Sequence
  Description of Artificial Sequence Synthetic peptide moiety
RSRWRDVARNFMR

SEQ ID NO: 111 - protein, 13 aa, Artificial Sequence
  Description of Artificial Sequence Synthetic peptide moiety
RSRWRDWSRNFMR

SEQ ID NO: 112 - protein, 13 aa, Artificial Sequence
  Description of Artificial Sequence Synthetic peptide moiety
RSRWRDVARNFMR

SEQ ID NO: 113 - protein, 13 aa, Artificial Sequence
  Description of Artificial Sequence Synthetic peptide moiety
RSRWRDWSRNFMR

SEQ ID NO: 114 - protein, 13 aa, Artificial Sequence
  Description of Artificial Sequence Synthetic peptide moiety
RSRWRDVARNFMR

SEQ ID NO: 115 - protein, 13 aa, Artificial Sequence
  Description of Artificial Sequence Synthetic peptide moiety
RSRWRDVSRNFMR

SEQ ID NO: 116 - protein, 13 aa, Artificial Sequence
  Description of Artificial Sequence Synthetic peptide moiety
RSRWRDWARNFMR

SEQ ID NO: 117 - protein, 13 aa, Artificial Sequence
  Description of Artificial Sequence Synthetic peptide moiety
RSRWRDWSRNFMR

SEQ ID NO: 118 - protein, 13 aa, Artificial Sequence
  Description of Artificial Sequence Synthetic peptide moiety
RSRWRDVARNFMR

SEQ ID NO: 119 - protein, 13 aa, Artificial Sequence
  Description of Artificial Sequence Synthetic peptide moiety
RSRWRDVARNFAR

SEQ ID NO: 120 - protein, 12 aa, Artificial Sequence
  Description of Artificial Sequence Synthetic peptide moiety
RSRWRDVARNFM

SEQ ID NO: 121 - protein, 11 aa, Artificial Sequence
  Description of Artificial Sequence Synthetic peptide moiety
RSRWRDVARNF

SEQ ID NO: 122 - protein, 10 aa, Artificial Sequence
  Description of Artificial Sequence Synthetic peptide moiety
RSRWRDVARN

SEQ ID NO: 123 - protein, 9 aa, Artificial Sequence
  Description of Artificial Sequence Synthetic peptide moiety
RSRWRDVAR

SEQ ID NO: 124 - protein, 8 aa, Artificial Sequence
  Description of Artificial Sequence Synthetic peptide moiety
RSRWRDVA

SEQ ID NO: 125 - protein, 7 aa, Artificial Sequence
  Description of Artificial Sequence Synthetic peptide moiety
RSRWRDV

SEQ ID NO: 126 - protein, 7 aa, Artificial Sequence
  Description of Artificial Sequence Synthetic peptide moiety
SRWRDVA

SEQ ID NO: 127 - protein, 7 aa, Artificial Sequence
  Description of Artificial Sequence Synthetic peptide moiety
RWRDVAR

SEQ ID NO: 128 - protein, 7 aa, Artificial Sequence
  Description of Artificial Sequence Synthetic peptide moiety
WRDVARN

SEQ ID NO: 129 - protein, 7 aa, Artificial Sequence
  Description of Artificial Sequence Synthetic peptide moiety
RDVARNF

SEQ ID NO: 130 - protein, 7 aa, Artificial Sequence
  Description of Artificial Sequence Synthetic peptide moiety
DVARNFM

SEQ ID NO: 131 - protein, 7 aa, Artificial Sequence
  Description of Artificial Sequence Synthetic peptide moiety
VARNFMR

SEQ ID NO: 132 - protein, 8 aa, Artificial Sequence
  Description of Artificial Sequence Synthetic peptide moiety
DVARNFMR

SEQ ID NO: 133 - protein, 9 aa, Artificial Sequence
  Description of Artificial Sequence Synthetic peptide moiety
RDVARNFMR

SEQ ID NO: 134 - protein, 10 aa, Artificial Sequence
  Description of Artificial Sequence Synthetic peptide moiety
WRDVARNFMR

SEQ ID NO: 135 - protein, 11 aa, Artificial Sequence
  Description of Artificial Sequence Synthetic peptide moiety
RWRDVARNFMR

SEQ ID NO: 136 - protein, 12 aa, Artificial Sequence
  Description of Artificial Sequence Synthetic peptide moiety
SRWRDVARNFMR

SEQ ID NO: 137 - protein, 12 aa, Artificial Sequence
  Description of Artificial Sequence Synthetic peptide moiety
RSRWRDVARNFM

SEQ ID NO: 138 - protein, 11 aa, Artificial Sequence
  Description of Artificial Sequence Synthetic peptide moiety
RSRWRDVARNF

SEQ ID NO: 139 - protein, 10 aa, Artificial Sequence
  Description of Artificial Sequence Synthetic peptide moiety
RSRWRDVARN

SEQ ID NO: 140 - protein, 9 aa, Artificial Sequence
  Description of Artificial Sequence Synthetic peptide moiety
RSRWRDVAR

SEQ ID NO: 141 - protein, 8 aa, Artificial Sequence
  Description of Artificial Sequence Synthetic peptide moiety
RSRWRDVA

SEQ ID NO: 142 - protein, 7 aa, Artificial Sequence
  Description of Artificial Sequence Synthetic peptide moiety
RSRWRDV

SEQ ID NO: 143 - protein, 7 aa, Artificial Sequence
  Description of Artificial Sequence Synthetic peptide moiety
SRWRDVA

SEQ ID NO: 144 - protein, 7 aa, Artificial Sequence
  Description of Artificial Sequence Synthetic peptide moiety
RWRDVAR

SEQ ID NO: 145 - protein, 7 aa, Artificial Sequence
  Description of Artificial Sequence Synthetic peptide moiety
WRDVARN

SEQ ID NO: 146 - protein, 7 aa, Artificial Sequence
  Description of Artificial Sequence Synthetic peptide moiety
RDVARNF

SEQ ID NO: 147 - protein, 7 aa, Artificial Sequence
  Description of Artificial Sequence Synthetic peptide moiety
DVARNFM

SEQ ID NO: 148 - protein, 7 aa, Artificial Sequence
  Description of Artificial Sequence Synthetic peptide moiety
VARNFMR

SEQ ID NO: 149 - protein, 8 aa, Artificial Sequence
  Description of Artificial Sequence Synthetic peptide moiety
DVARNFMR

SEQ ID NO: 150 - protein, 9 aa, Artificial Sequence
  Description of Artificial Sequence Synthetic peptide moiety
RDVARNFMR

SEQ ID NO: 151 - protein, 10 aa, Artificial Sequence
  Description of Artificial Sequence Synthetic peptide moiety
WRDVARNFMR

SEQ ID NO: 152 - protein, 11 aa, Artificial Sequence
  Description of Artificial Sequence Synthetic peptide moiety
RWRDVARNFMR

SEQ ID NO: 153 - protein, 12 aa, Artificial Sequence
  Description of Artificial Sequence Synthetic peptide moiety
SRWRDVARNFMR

SEQ ID NO: 154 - protein, 13 aa, Artificial Sequence
  Description of Artificial Sequence Synthetic peptide moiety
RSRLRDLLRNLAR

SEQ ID NO: 155 - protein, 13 aa, Artificial Sequence
  Description of Artificial Sequence Synthetic peptide moiety
RSRWRDLLRNLMR

SEQ ID NO: 156 - protein, 13 aa, Artificial Sequence
  Description of Artificial Sequence Synthetic peptide moiety
RSRWRDVLRNFMR

SEQ ID NO: 157 - protein, 13 aa, Artificial Sequence
  Description of Artificial Sequence Synthetic peptide moiety
RSRWRDVARNFLR

SEQ ID NO: 158 - protein, 13 aa, Artificial Sequence
  Description of Artificial Sequence Synthetic peptide moiety
RSRWRDVLRNFLR

SEQ ID NO: 159 - protein, 15 aa, Artificial Sequence
  Description of Artificial Sequence Synthetic peptide moiety
RSRWRDVARNFMRLL

SEQ ID NO: 160 - protein, 15 aa, Artificial Sequence
  Description of Artificial Sequence Synthetic peptide moiety
RSRWRDVLRNFLRLL

SEQ ID NO: 161 - protein, 13 aa, Artificial Sequence
  Description of Artificial Sequence Synthetic peptide moiety
RSRWRDVARNWMR

SEQ ID NO: 162 - protein, 13 aa, Artificial Sequence
  Description of Artificial Sequence Synthetic peptide moiety
RSRWRDWARNFMR

SEQ ID NO: 163 - protein, 14 aa, Artificial Sequence
  Description of Artificial Sequence Synthetic peptide moiety
RSRWRDVARNFMRW

SEQ ID NO: 164 - protein, 15 aa, Artificial Sequence
  Description of Artificial Sequence Synthetic peptide moiety
RSRWRDVARNFMRWW

SEQ ID NO: 165 - protein, 13 aa, Artificial Sequence
  Description of Artificial Sequence Synthetic peptide moiety
RSRWRDWARNWMR

SEQ ID NO: 166 - protein, 14 aa, Artificial Sequence
  Description of Artificial Sequence Synthetic peptide moiety
RSRWRDWARNWMRW

SEQ ID NO: 167 - protein, 15 aa, Artificial Sequence
  Description of Artificial Sequence Synthetic peptide moiety
RSRWRDWARNWMRWW

SEQ ID NO: 168 - protein, 15 aa, Artificial Sequence
  Description of Artificial Sequence Synthetic peptide moiety
RSRWRDWARNWMRLL

SEQ ID NO: 169 - protein, 13 aa, Artificial Sequence
  Description of Artificial Sequence Synthetic peptide moiety
RSRWRDWWRNWMR

SEQ ID NO: 170 - protein, 15 aa, Artificial Sequence
  Description of Artificial Sequence Synthetic peptide moiety
RSRWRDWWRNWMRLL

SEQ ID NO: 171 - protein, 13 aa, Artificial Sequence
  Description of Artificial Sequence Synthetic peptide moiety
RSRXRDVARNFMR

SEQ ID NO: 172 - protein, 13 aa, Artificial Sequence
  Description of Artificial Sequence Synthetic peptide moiety
RSRWRDXARNFMR

SEQ ID NO: 173 - protein, 13 aa, Artificial Sequence
  Description of Artificial Sequence Synthetic peptide moiety
RSRWRDVXRNFMR

SEQ ID NO: 174 - protein, 13 aa, Artificial Sequence
  Description of Artificial Sequence Synthetic peptide moiety
RSRWRDVARNXMR

SEQ ID NO: 175 - protein, 13 aa, Artificial Sequence
  Description of Artificial Sequence Synthetic peptide moiety
RSRXRDXARNFMR

SEQ ID NO: 176 - protein, 13 aa, Artificial Sequence
  Description of Artificial Sequence Synthetic peptide moiety
RSRXRDVARNXMR

SEQ ID NO: 177 - protein, 13 aa, Artificial Sequence
  Description of Artificial Sequence Synthetic peptide moiety
RSRXRDXARNXMR

SEQ ID NO: 178 - protein, 13 aa, Artificial Sequence
  Description of Artificial Sequence Synthetic peptide moiety
RSRWRDXARNXMR

SEQ ID NO: 179 - protein, 15 aa, Artificial Sequence
  Description of Artificial Sequence Synthetic peptide moiety
RSRXRDXARNXMRXX

SEQ ID NO: 180 - protein, 13 aa, Artificial Sequence
  Description of Artificial Sequence Synthetic peptide moiety
RSRWRDVARNFMR

SEQ ID NO: 181 - protein, 13 aa, Artificial Sequence
  Description of Artificial Sequence Synthetic peptide moiety
XSXWXDVAXNFMX

SEQ ID NO: 182 - protein, 14 aa, Artificial Sequence
  Description of Artificial Sequence Synthetic peptide moiety
XSXXXDXAXNXMXX

SEQ ID NO: 183 - protein, 12 aa, Artificial Sequence
  Description of Artificial Sequence Synthetic peptide moiety
RSRWRDVARNFL

SEQ ID NO: 184 - protein, 12 aa, Artificial Sequence
  Description of Artificial Sequence Synthetic peptide moiety
SRWRDVARNFLR

SEQ ID NO: 185 - protein, 11 aa, Artificial Sequence
  Description of Artificial Sequence Synthetic peptide moiety
RWRDVARNFLR

SEQ ID NO: 186 - protein, 13 aa, Artificial Sequence
  Description of Artificial Sequence Synthetic peptide moiety
RSRWRDVARNFLR

SEQ ID NO: 187 - protein, 13 aa, Artificial Sequence
  Description of Artificial Sequence Synthetic peptide moiety
RSRWRDVARNFLR

SEQ ID NO: 188 - protein, 13 aa, Artificial Sequence
  Description of Artificial Sequence Synthetic peptide moiety
RSRWRDVARNFLR

SEQ ID NO: 189 - protein, 13 aa, Artificial Sequence
  Description of Artificial Sequence Synthetic peptide moiety
RSRWRDVARNFLR

SEQ ID NO: 190 - protein, 13 aa, Artificial Sequence
  Description of Artificial Sequence Synthetic peptide moiety
RSRWRDVARNFLR

SEQ ID NO: 191 - protein, 13 aa, Artificial Sequence
  Description of Artificial Sequence Synthetic peptide moiety
RSRWRDVARNFMR

SEQ ID NO: 192 - protein, 13 aa, Artificial Sequence
  Description of Artificial Sequence Synthetic peptide moiety
RSRWRDVARNFMR

SEQ ID NO: 193 - protein, 13 aa, Artificial Sequence
  Description of Artificial Sequence Synthetic peptide moiety
RSRWRDVARNFMR

SEQ ID NO: 194 - protein, 13 aa, Artificial Sequence
  Description of Artificial Sequence Synthetic peptide moiety
RSRWRDVARNFMR

SEQ ID NO: 195 - protein, 13 aa, Artificial Sequence
  Description of Artificial Sequence Synthetic peptide moiety
RSRWRDVARNFMR

SEQ ID NO: 196 - protein, 14 aa, Artificial Sequence
  Description of Artificial Sequence Synthetic peptide moiety
RSRWRDVARNFLRK

SEQ ID NO: 197 - protein, 14 aa, Artificial Sequence
  Description of Artificial Sequence Synthetic peptide moiety
RSRWRDVARNFLRK

SEQ ID NO: 198 - protein, 14 aa, Artificial Sequence
  Description of Artificial Sequence Synthetic peptide moiety
RSRWRDVARNFLRK

SEQ ID NO: 199 - protein, 14 aa, Artificial Sequence
  Description of Artificial Sequence Synthetic peptide moiety
RSRWRDVARNFLRK

SEQ ID NO: 200 - protein, 14 aa, Artificial Sequence
  Description of Artificial Sequence Synthetic peptide moiety
RSRWRDVARNFLRK

SEQ ID NO: 201 - protein, 14 aa, Artificial Sequence
  Description of Artificial Sequence Synthetic peptide moiety
RSRWRDVARNFMRK

SEQ ID NO: 202 - protein, 14 aa, Artificial Sequence
  Description of Artificial Sequence Synthetic peptide moiety
RSRWRDVARNFMRK

SEQ ID NO: 203 - protein, 14 aa, Artificial Sequence
  Description of Artificial Sequence Synthetic peptide moiety
RSRWRDVARNFMRK

SEQ ID NO: 204 - protein, 14 aa, Artificial Sequence
  Description of Artificial Sequence Synthetic peptide moiety
RSRWRDVARNFMRK

SEQ ID NO: 205 - protein, 14 aa, Artificial Sequence
  Description of Artificial Sequence Synthetic peptide moiety
RSRWRDVARNFMRK

SEQ ID NO: 206 - protein, 13 aa, Artificial Sequence
  Description of Artificial Sequence Synthetic peptide moiety
RSRWRDVARNFLR

SEQ ID NO: 207 - protein, 13 aa, Artificial Sequence
  Description of Artificial Sequence Synthetic peptide moiety
RSRWRDVARNFLR

SEQ ID NO: 208 - protein, 13 aa, Artificial Sequence
  Description of Artificial Sequence Synthetic peptide moiety
RSRWRDVARNFLR

SEQ ID NO: 209 - protein, 13 aa, Artificial Sequence
  Description of Artificial Sequence Synthetic peptide moiety
RSRWRDVARNFLR

SEQ ID NO: 210 - protein, 14 aa, Artificial Sequence
  Description of Artificial Sequence Synthetic peptide moiety
RSRWRDVARNFLRK

SEQ ID NO: 211 - protein, 14 aa, Artificial Sequence
  Description of Artificial Sequence Synthetic peptide moiety
RSRWRDVARNFLRK

SEQ ID NO: 212 - protein, 14 aa, Artificial Sequence
  Description of Artificial Sequence Synthetic peptide moiety
RSRWRDVARNFLRK

SEQ ID NO: 213 - protein, 14 aa, Artificial Sequence
  Description of Artificial Sequence Synthetic peptide moiety
RSRWRDVARNFLRK

SEQ ID NO: 214 - protein, 14 aa, Artificial Sequence
  Description of Artificial Sequence Synthetic peptide moiety
RSRWRDVARNFLRK

SEQ ID NO: 215 - protein, 14 aa, Artificial Sequence
  Description of Artificial Sequence Synthetic peptide moiety
RSRWRDVARNFMRK

SEQ ID NO: 216 - protein, 14 aa, Artificial Sequence
  Description of Artificial Sequence Synthetic peptide moiety
RSRWRDVARNFMRK

SEQ ID NO: 217 - protein, 14 aa, Artificial Sequence
  Description of Artificial Sequence Synthetic peptide moiety
RSRWRDVARNFMRK

SEQ ID NO: 218 - protein, 14 aa, Artificial Sequence
  Description of Artificial Sequence Synthetic peptide moiety
RSRWRDVARNFMRK

SEQ ID NO: 219 - protein, 14 aa, Artificial Sequence
  Description of Artificial Sequence Synthetic peptide moiety
RSRWRDVARNFMRK

SEQ ID NO: 220 - protein, 13 aa, Artificial Sequence
  Description of Artificial Sequence Synthetic peptide moiety
RSRWRDVARNFMR

SEQ ID NO: 221 - protein, 13 aa, Artificial Sequence
  Description of Artificial Sequence Synthetic peptide moiety
RSRWRDVARNFMR

SEQ ID NO: 222 - protein, 13 aa, Artificial Sequence
  Description of Artificial Sequence Synthetic peptide moiety
RSRWRDVARNFMR

SEQ ID NO: 223 - protein, 13 aa, Artificial Sequence
  Description of Artificial Sequence Synthetic peptide moiety
RSRWRDVARNFMR

SEQ ID NO: 224 - protein, 13 aa, Artificial Sequence
  Description of Artificial Sequence Synthetic peptide moiety
RSRWRDVARNFMR

SEQ ID NO: 225 - protein, 13 aa, Artificial Sequence
  Description of Artificial Sequence Synthetic peptide moiety
RSRWRDVARNFMR

SEQ ID NO: 226 - protein, 13 aa, Artificial Sequence
  Description of Artificial Sequence Synthetic peptide moiety
RSRWRDVARNFMR

SEQ ID NO: 227 - protein, 13 aa, Artificial Sequence
  Description of Artificial Sequence Synthetic peptide moiety
RSRWRDVARNFMR

SEQ ID NO: 228 - protein, 13 aa, Artificial Sequence
  Description of Artificial Sequence Synthetic peptide moiety
RSRWRDVARNFLR

SEQ ID NO: 229 - protein, 12 aa, Artificial Sequence
  Description of Artificial Sequence Synthetic peptide moiety
RSRWRDVARNFL

SEQ ID NO: 230 - protein, 12 aa, Artificial Sequence
  Description of Artificial Sequence Synthetic peptide moiety
SRWRDVARNFLR

SEQ ID NO: 231 - protein, 13 aa, Artificial Sequence
  Description of Artificial Sequence Synthetic peptide moiety
RSRWRDVARNFLR

SEQ ID NO: 232 - protein, 13 aa, Artificial Sequence
  Description of Artificial Sequence Synthetic peptide moiety
RWSRVDRANRFLR

SEQ ID NO: 233 - protein, 13 aa, Artificial Sequence
  Description of Artificial Sequence Synthetic peptide moiety
RWSRVDRANRFLR

SEQ ID NO: 234 - protein, 13 aa, Artificial Sequence
  Description of Artificial Sequence Synthetic peptide moiety
RWSRIDRINRFLR

SEQ ID NO: 235 - protein, 13 aa, Artificial Sequence
  Description of Artificial Sequence Synthetic peptide moiety
RARWRAVARAFAR

SEQ ID NO: 236 - protein, 13 aa, Artificial Sequence
  Description of Artificial Sequence Synthetic peptide moiety
RLRWRLVLRLFLR

SEQ ID NO: 237 - protein, 13 aa, Artificial Sequence
  Description of Artificial Sequence Synthetic peptide moiety
RKRWRDVARNFLR

SEQ ID NO: 238 - protein, 13 aa, Artificial Sequence
  Description of Artificial Sequence Synthetic peptide moiety
RKRWREVARNFLR

SEQ ID NO: 239 - protein, 13 aa, Artificial Sequence
  Description of Artificial Sequence Synthetic peptide moiety
RSRWREVARNFLR

SEQ ID NO: 240 - protein, 13 aa, Artificial Sequence
  Description of Artificial Sequence Synthetic peptide moiety
RRRWRRVARRFLR

SEQ ID NO: 241 - protein, 13 aa, Artificial Sequence
  Description of Artificial Sequence Synthetic peptide moiety
RKRWRKVARKFLR

SEQ ID NO: 242 - protein, 13 aa, Artificial Sequence
  Description of Artificial Sequence Synthetic peptide moiety
RARWRDVARNFLR

SEQ ID NO: 243 - protein, 13 aa, Artificial Sequence
  Description of Artificial Sequence Synthetic peptide moiety
RSRWRAVARNFLR

SEQ ID NO: 244 - protein, 13 aa, Artificial Sequence
  Description of Artificial Sequence Synthetic peptide moiety
RSRWRDVARAFLR

SEQ ID NO: 245 - protein, 13 aa, Artificial Sequence
  Description of Artificial Sequence Synthetic peptide moiety
RLRWREVARLFLR

SEQ ID NO: 246 - protein, 13 aa, Artificial Sequence
  Description of Artificial Sequence Synthetic peptide moiety
RSAWRDVARNFMR

SEQ ID NO: 247 - protein, 13 aa, Artificial Sequence
  Description of Artificial Sequence Synthetic peptide moiety
RSRWADVARNFMR

SEQ ID NO: 248 - protein, 13 aa, Artificial Sequence
  Description of Artificial Sequence Synthetic peptide moiety
RSRWRDVAANFMR

SEQ ID NO: 249 - protein, 13 aa, Artificial Sequence
  Description of Artificial Sequence Synthetic peptide moiety
RSRWRDVARNFMA

SEQ ID NO: 250 - protein, 13 aa, Artificial Sequence
  Description of Artificial Sequence Synthetic peptide moiety
RSRSRDVARNFMR

SEQ ID NO: 251 - protein, 13 aa, Artificial Sequence
  Description of Artificial Sequence Synthetic peptide moiety
RSRWRDSARNFMR

SEQ ID NO: 252 - protein, 13 aa, Artificial Sequence
  Description of Artificial Sequence Synthetic peptide moiety
RSRWRDVARNSMR

SEQ ID NO: 253 - protein, 13 aa, Artificial Sequence
  Description of Artificial Sequence Synthetic peptide moiety
RSRWRDVARNFSR

SEQ ID NO: 254 - protein, 13 aa, Artificial Sequence
  Description of Artificial Sequence Synthetic peptide moiety
ASRWRDVARNFMR

SEQ ID NO: 255 - protein, 13 aa, Artificial Sequence
  Description of Artificial Sequence Synthetic peptide moiety
RARWRDVARNFMR

SEQ ID NO: 256 - protein, 13 aa, Artificial Sequence
  Description of Artificial Sequence Synthetic peptide moiety
RSAWRDVARNFMR

SEQ ID NO: 257 - protein, 13 aa, Artificial Sequence
  Description of Artificial Sequence Synthetic peptide moiety
RSRARDVARNFMR

SEQ ID NO: 258 - protein, 13 aa, Artificial Sequence
  Description of Artificial Sequence Synthetic peptide moiety
RSRWADVARNFMR

SEQ ID NO: 259 - protein, 7 aa, Artificial Sequence
  Description of Artificial Sequence Synthetic peptide moiety
RWRDLAR

SEQ ID NO: 260 - protein, 7 aa, Artificial Sequence
  Description of Artificial Sequence Synthetic peptide moiety
RWRALAR

SEQ ID NO: 261 - protein, 7 aa, Artificial Sequence
  Description of Artificial Sequence Synthetic peptide moiety
RWRAVAR

SEQ ID NO: 262 - protein, 7 aa, Artificial Sequence
  Description of Artificial Sequence Synthetic peptide moiety
RWRDLAR

SEQ ID NO: 263 - protein, 7 aa, Artificial Sequence
  Description of Artificial Sequence Synthetic peptide moiety
RWRALAR

SEQ ID NO: 264 - protein, 7 aa, Artificial Sequence
  Description of Artificial Sequence Synthetic peptide moiety
RWRAVAR

SEQ ID NO: 265 - protein, 14 aa, Artificial Sequence
  Description of Artificial Sequence Synthetic peptide moiety
RSRWRDVARNFMRC

SEQ ID NO: 266 - protein, 13 aa, Artificial Sequence
  Description of Artificial Sequence Synthetic peptide moiety
RSRWRDVARNFMR

SEQ ID NO: 267 - protein, 13 aa, Artificial Sequence
  Description of Artificial Sequence Synthetic peptide moiety
RSRWRDVARDFMR

SEQ ID NO: 268 - protein, 19 aa, Artificial Sequence
  Description of Artificial Sequence Synthetic peptide moiety
TRVARTGRSRWRDWARNFM

SEQ ID NO: 269 - protein, 13 aa, Artificial Sequence
  Description of Artificial Sequence Synthetic peptide moiety
RSRWRDVARNFLR

SEQ ID NO: 270 - protein, 13 aa, Artificial Sequence
  Description of Artificial Sequence Synthetic peptide moiety
RSRWRDVARNFLR

SEQ ID NO: 271 - protein, 13 aa, Artificial Sequence
  Description of Artificial Sequence Synthetic peptide moiety
RSRWRDVARNFLR

SEQ ID NO: 272 - protein, 13 aa, Artificial Sequence
  Description of Artificial Sequence Synthetic peptide moiety
RSRWRDVARNFLR

SEQ ID NO: 273 - protein, 8 aa, Artificial Sequence
  Description of Artificial Sequence Synthetic peptide moiety
RWRDVARN

SEQ ID NO: 274 - protein, 8 aa, Artificial Sequence
  Description of Artificial Sequence Synthetic peptide moiety
SRWRDVAR

SEQ ID NO: 275 - protein, 9 aa, Artificial Sequence
  Description of Artificial Sequence Synthetic peptide moiety
SRWRDVARN

SEQ ID NO: 276 - protein, 9 aa, Artificial Sequence
  Description of Artificial Sequence Synthetic peptide moiety
RWRDVARNF

SEQ ID NO: 277 - protein, 10 aa, Artificial Sequence
  Description of Artificial Sequence Synthetic peptide moiety
SRWRDVARNF

SEQ ID NO: 278 - protein, 10 aa, Artificial Sequence
  Description of Artificial Sequence Synthetic peptide moiety
RWRDVARNFM

SEQ ID NO: 279 - protein, 7 aa, Artificial Sequence
  Description of Artificial Sequence Synthetic peptide
GDDDDDD

SEQ ID NO: 280 - protein, 21 aa, Artificial Sequence
  Description of Artificial Sequence Synthetic peptide moiety
TRVSRTGRSRWRDWSRNFMRK

SEQ ID NO: 281 - protein, 12 aa, Artificial Sequence
  Description of Artificial Sequence Synthetic peptide
RRQRRTSKLMKR

SEQ ID NO: 282 - protein, 8 aa, Artificial Sequence
  Description of Artificial Sequence Synthetic polylysine peptide
KKKKKKKK

SEQ ID NO: 283 - protein, 13 aa, Artificial Sequence
  Description of Artificial Sequence Synthetic peptide moiety
RSRWRDVARNFMR

SEQ ID NO: 284 - protein, 7 aa, Artificial Sequence
  Description of Artificial Sequence Synthetic peptide moiety
RWRDVAR

SEQ ID NO: 285 - protein, 8 aa, Artificial Sequence
  Description of Artificial Sequence Synthetic peptide moiety
RSRWRDVA

SEQ ID NO: 286 - protein, 9 aa, Artificial Sequence
  Description of Artificial Sequence Synthetic peptide moiety
RSRWRDVAR

SEQ ID NO: 287 - protein, 9 aa, Artificial Sequence
  Description of Artificial Sequence Synthetic peptide moiety
RDVARNFMR

SEQ ID NO: 288 - protein, 10 aa, Artificial Sequence
  Description of Artificial Sequence Synthetic peptide moiety
RSRWRDVARN

SEQ ID NO: 289 - protein, 10 aa, Artificial Sequence
  Description of Artificial Sequence Synthetic peptide moiety
WRDVARNFMR

SEQ ID NO: 290 - protein, 11 aa, Artificial Sequence
  Description of Artificial Sequence Synthetic peptide moiety
RSRWRDVARNF

SEQ ID NO: 291 - protein, 11 aa, Artificial Sequence
  Description of Artificial Sequence Synthetic peptide moiety
SRWRDVARNFM

SEQ ID NO: 292 - protein, 11 aa, Artificial Sequence
  Description of Artificial Sequence Synthetic peptide moiety
RWRDVARNFMR

SEQ ID NO: 293 - protein, 12 aa, Artificial Sequence
  Description of Artificial Sequence Synthetic peptide moiety
RSRWRDVARNFM

SEQ ID NO: 294 - protein, 12 aa, Artificial Sequence
  Description of Artificial Sequence Synthetic peptide moiety
SRWRDVARNFMR

(19) United States
(12) Patent Application Publication
(10) Pub. No.: US 2005/0282755 A1
(43) Pub. Date: Dec. 22, 2005

(54) Compositions having antimicrobial activity and uses thereof

(75) Inventors: Scott A. Hart, San Diego, CA (US); Karin Zeh, San Diego, CA (US); Thomas Machleidt, Madison, WI (US)

Correspondence Address: BIOTECHNOLOGY LAW GROUP, C/O PORTFOLIOIP, P.O. BOX 52050, MINNEAPOLIS, MN, 55402, US

(73) Assignee: Ansata Therapeutics, Inc., La Jolla, CA (US)

(21) Appl. No.: 11/078,469

(22) Filed: Mar. 11, 2005

Related U.S. Application Data
(60) Provisional application No. 60/618,948, filed on Oct. 15, 2004.
(60) Provisional application No. 60/554,526, filed on Mar. 18, 2004.

(51) Int. Cl. A61K 38/10

(52) U.S. Cl. 514/14

(57) ABSTRACT

Provided are compositions and methods useful for reducing microbial populations on and/or in skin, reducing skin inflammation, targeting skin substructures and components, and treating skin conditions such as acne. A composition often comprises an antimicrobial peptidyl moiety having an amino acid sequence conforming to a sequence motif provided herein, and sometimes derived from the polypeptide granulysin. The composition optionally comprises a lipophilic moiety that increases the hydrophobicity of the peptidyl moiety, which may target the composition to specific areas of skin in a subject to whom the composition is administered. Also featured are apparatus useful for testing peptide compositions for biological activity on and/or in skin.

### RELATED PATENT APPLICATIONS

This patent application claims the benefit of U.S. Provisional Patent Application No. 60/554,526 filed on Mar. 18, 2004 and Ser. No. 60/618,948 filed on Oct. 15, 2004, each of naming Scott A. Hart et al. as inventors and entitled “Compositions Having Antimicrobial Activity and Uses Thereof.” Each of these patent applications is incorporated herein by reference in its entirety, including all tables and drawings.

### FIELD OF THE INVENTION

The invention relates to antimicrobial peptide compositions and their uses. Uses include reducing microbial populations on and/or in skin, reducing skin inflammation, targeting skin substructures and components (e.g., sebum, a sebaceous gland, an open or closed comedone, an open or closed pore and/or a pilosebaceous unit), and treating a skin condition, such as acne for example.

### BACKGROUND

Mammalian skin is a multifunctional organ that protects the body and performs several specialized functions, such as breathing, perspiring, sensory information processing, and oil production. Oil production, essential to the protective features of the skin, is the release of an oily substance known as sebum from the sebaceous glands, which are large glands located at the base of a hair follicle. Sebum production permits the skin to moisturize and waterproof itself, thereby protecting itself from the environment.

Puberty often gives rise to increased sebum production, which in some cases is caused by increased levels of testosterone in males and females. Also, testosterone causes cells lining pores in the skin to release more keratin, an insoluble protein that is the primary constituent of hair and the epidermis. Excess sebum production is an important factor in the development of acne, which usually doesn't occur until puberty. Development of acne is dependent on multiple factors: plugging of the hair follicle with desquamated cells, increased sebum production, proliferation of P. acnes and the initiation of an inflammatory response. Plugging of the hair follicles leads to accumulation of sebum and formation of microcomedones. Continuous production of sebum and enlargement of a microcomedone results in a visible closed comedone (whitehead). In this environment, resident bacteria, predominantly P. acnes, convert triglycerides in sebum into glycerol and free fatty acids, which initiate an inflammatory response. Furthermore, the release of proinflammmtory and chemotactic factors from P. acnes exacerbates the inflammation, which eventually can result in follicular wall rupture and formation of an inflammatory acne lesion. The degree of the inflammatory response correlates well with the severity of clinical symptoms of acne.

This inflammation often results in pustules or pimples and results in skin conditions such as acne vulgaris. This condition is especially prevalent on the face, back, and shoulders, where a greater number of sebaceous glands exist. Acne conglobate, more commonly known as nodular or cystic acne, is a more severe form of acne. In the case of nodular acne, sebum accumulates in glands, mixes with dead cells, and eventually ruptures follicle walls, which typically leads to a deep cyst under the skin. Scarring often results from these deep cysts.

Acne is a skin disease that often scars those afflicted, and can afflict patients at young ages, typically in teen years, when their self-images are the most sensitive. Thus, acne not only affects a person's appearance, but sometimes has detrimental affects on the person's psychological, social, and occupational status. The scarring commonly is permanent even if the condition is treated with medications. Some patients experience symptoms well into their adult years.

### SUMMARY

Relatively short peptides having antimicrobial activity have been discovered. These peptides sometimes are linked to a lipophilic moiety, often are formulated in compositions comprising a pharmaceutically acceptable carrier, and often are utilized in antimicrobial methods, such as reducing a microbial population on and/or in skin; methods for reducing skin inflammation; methods for targeting one or more skin substructures or components (e.g., sebum, keratin, one or more sebaceous glands, one or more open pores and/or blocked pores, one or more open comedones and/or closed comedones, one or more pilosebaceous units); and methods for treating a skin condition such as acne, for example.

Thus, provided is a peptide composition having antimicrobial properties that comprises, consists essentially of, or consists of an amino acid sequence conforming to a sequence motif pattern in Table 1.

TABLE 1

| | | |
|---|---|---|
| | | SEQ |
| | | ID |
| Motif | Motif Sequence | NO: |

| | | |
|---|---|---|
| I | -B₁-X₁-B₂-Z₁-B₃-X₂-Z₂-X₃- | 1 |
| II | -B₁-X₁-B₂-Z₁-B₃-X₂-Z₂-X₃-B₄- | 2 |
| III | -B₁-X₁-B₂-Z₁-B₃-X₂-Z₂-X₃-B₄-X₄- | 3 |
| IV | -B₁-X₁-B₂-Z₁-B₃-X₂-Z₂-X₃-B₄-X₄-Z₃- | 4 |
| V | -B₁-X₁-B₂-Z₁-B₃-X₂-Z₂-X₃-B₄-X₄-Z₃-X₅- | 5 |
| VI | -B₁-X₁-B₂-Z₁-B₃-X₂-Z₂-X₃-B₄-X₄-Z₃-X₅-B₅- | 6 |
| VII | -X₁-B₂-Z₁-B₃-X₂-Z₂-X₃-B₄- | 7 |
| VIII | -X₁-B₂-Z₁-B₃-X₂-Z₂-X₃-B₄-X₄- | 8 |
| IX | -X₁-B₂-Z₁-B₃-X₂-Z₂-X₃-B₄-X₄-Z₃- | 9 |
| X | -X₁-B₂-Z₁-B₃-X₂-Z₂-X₃-B₄-X₄-Z₃-X₅- | 10 |
| XI | -X₁-B₂-Z₁-B₃-X₂-Z₂-X₃-B₄-X₄-Z₃-X₅-B₅- | 11 |
| XII | -B₂-Z₁-B₃-X₂-Z₂-X₃-B₄- | 12 |
| XIII | -B₂-Z₁-B₃-X₂-Z₂-X₃-B₄-X₄- | 13 |
| XIV | -B₂-Z₁-B₃-X₂-Z₂-X₃-B₄-X₄-Z₃- | 14 |
| XV | -B₂-Z₁-B₃-X₂-Z₂-X₃-B₄-X₄-Z₃-X₅- | 15 |
| XVI | -B₂-Z₁-B₃-X₂-Z₂-X₃-B₄-X₄-Z₃-X₅-B₅- | 16 |
| XVII | -Z₁-B₃-X₂-Z₂-X₃-B₄-X₄-Z₃-X₅-B₅- | 17 |
| XVIII | -B₃-X₂-Z₂-X₃-B₄-X₄-Z₃-X₅-B₅- | 18 |

In Table 1, B₁, B₂, B₃, B₄ and B₅ are independently selected from basic amino acids, Z₁, Z₂ and Z₃ are independently selected from hydrophobic amino acids, and X₁, X₂, X₃, X₄ and X₅ are independently selected from any amino acid. Amino acids in each peptide composition include, but are not limited to, D-amino acids, L-amino acids, natural amino acids, unnatural or non-classical amino acids, and/or alpha amino acid homologs (e.g., beta²-, beta³- and/or gamma-amino acids). In certain embodiments, the peptide comprises or consists of all D-amino acids, all L-amino acids, a mixture of D- and L-amino acids, all natural amino acids, all unnatural or non-classical amino acids, all alpha amino acid homologs, a mixture of natural amino acids and unnatural or non-classical amino acids, a mixture of natural amino acids and alpha amino acid homologs, and a mixture of unnatural or non-classical amino acids and alpha amino acid homologs. In specific embodiments, the amino acid sequence of the peptide comprises or consists of a subsequence of a native granulysin antimicrobial protein, or a variant amino acid sequence thereof. The amino acid sequence of the peptide composition often comprises, consists of, or consists essentially of an amino acid sequence conforming to one of motifs I to XVIII, and sometimes each end position of a motif designates the N-terminus and C-terminus of the amino acid sequence (e.g., the N-terminal boundary of the amino acid sequence may be formed by B₁ and the C-terminal boundary of the amino acid sequence may be formed by X₃ for a peptide composition amino acid sequence conforming to motif 1).

In some embodiments, the composition contains a peptide moiety comprising an amino acid sequence conforming to sequence motif I, II, III, IV, V, VI, VII, VIII, IX, X, XI, XII, XIII, XIV, XV, XVI, XVII or XVIII, where B₁, B₂, B₃, B₄ and B₅ are independently selected from basic amino acids; Z₁, Z₂ and Z₃ are independently selected from hydrophobic amino acids; X₁, X₄ and X₅ are independently selected from hydrophobic amino acids, neutral hydrophilic amino acids, and acidic amino acids; and X₂ and X₃ are independently selected from hydrophobic amino acids and acidic amino acids. In certain embodiments, X₁ sometimes is a hydrophobic amino acid or an acidic amino acid; X₁ sometimes is a hydrophobic amino acid; X₂ sometimes is a hydrophobic amino acid; X₂ sometimes is an acidic amino acid; X₃ sometimes is a hydrophobic amino acid; X₃ sometimes is an acidic amino acid; X₄ sometimes is a hydrophobic amino acid or an acidic amino acid; X₄ sometimes is an acidic amino acid; X₄ sometimes is a hydrophobic amino acid; X₅ sometimes is an acidic amino acid; X₅ sometimes is a hydrophobic amino acid; two or more or three or more of X₁, X₂, X₃, X₄ and X₅ sometimes are independently selected from hydrophobic amino acids; X₁, X₂, X₃, X₄ and X₅ sometimes are independently selected from hydrophobic amino acids; the hydrophobic amino acids sometimes are independently selected from alanine and leucine; the hydrophobic amino acids sometimes are alanine; the hydrophobic amino acids sometimes are leucine; and combinations of the forgoing. In some embodiments, the amino acid sequence does not include two, three, four, or five or more consecutive basic amino acids. In certain embodiments, the amino acid sequence does not include a cysteine. In some embodiments, the amino acid sequence does not include the following “R-Z” sequences: -Z-R-R-Z-Z-R-; -Z-R-R-Z-R-R-Z; Z-Z-R-R-Z-R-R-Z-; -R-Z-Z-R-R-R-Z-R-R-Z-; -R-Z-Z-R-R-Z-R-R-Z-; -R-R-R-Z-Z-R-R-Z-; where Z is a hydrophobic amino acid and R is a basic or neutral hydrophilic amino acid. In related embodiments, the amino acid sequence of the peptide moiety may include one of the “R-Z” sequences in the foregoing sentence, where the N-terminal or C-terminal amino acid in one of the “R-Z” sequences is the terminal amino acid in the peptide moiety amino acid sequence.

The peptide moiety in the composition sometimes is about 7 to about 40 amino acids, about 7 to about 25 amino acids, or about 7 to about 20 amino acids in length, sometimes is about 7 to about 19 amino acids, about 7 to about 15 amino acids, about 7 to about 14 amino acids, about 7 to about 13 amino acids, about 8 to about 15 amino acids, about 8 to about 14 amino acids, about 8 to about 13 amino acids, about 9 to about 15 amino acids, about 9 to about 14 amino acids, about 9 to about 13 amino acids, about 10 to about 15 amino acids, about 10 to about 14 amino acids, about 10 to about 13 amino acids, about 11 to about 15 amino acids, about 11 to about 14 amino acids, about 11 to about 13 amino acids or about 13 amino acids in length, and sometimes is about 7, 8, 9, 10, 11, 12, 13, 14, 15, 16, 17, 18, 19, 20, 21, 22, 23, 24, 25, 26, 27, 28, 29 or 30 amino acids in length. The peptide moiety often does not form a helix-turn-helix structure and sometimes does not substantially form a helical structure in an aqueous solution or in a pharmaceutical formulation (described hereafter). In some embodiments, all of the amino acids in the peptide moiety are L-isomer amino acids; all of the amino acids in the peptide moiety are D-isomer amino acids; or the peptide moiety is a mixture of L-isomer and D-isomer amino acids. The composition sometimes comprises a lipophilic molecule linked to the peptide moiety.

In some embodiments, the composition contains a peptide moiety about 13 to about 15 amino acids in length comprising the amino acid sequence RSRWRDVARNFMR or an amino acid variant thereof. In some embodiments, the composition contains a peptide moiety about 7 to about 15 amino acids in length comprising the amino acid sequence RWRDVAR or an amino acid variant thereof. The composition sometimes contains a peptide moiety about 8 to about 15 amino acids in length comprising the amino acid sequence RSRWRDVA, RWRDVARN, SRWRDVAR or an amino acid variant thereof. In some embodiments the composition contains a peptide moiety about 9 to about 15 amino acids in length comprising the amino acid sequence RSRWRDVAR, SRWRDVARN, RWRDVARNF, RDVARNFMR or an amino acid variant thereof. A composition sometimes contains a peptide moiety about 10 to about 15 amino acids in length comprising the amino acid sequence RSRWRDVARN, SRWRDVARNF, RWRDVARNFM, WRDVARNFMR or an amino acid variant thereof. In some embodiments, a composition contains a peptide moiety about 11 to about 15 amino acids in length comprising the amino acid sequence RSRWRDVARNF, SRWRDVARNFM, RWRDVARNFMR or an amino acid variant thereof. A composition sometimes comprises a peptide moiety about 12 to about 15 amino acids in length comprising the amino acid sequence RSRWRDVARNFM, SRWRDVARNFMR or an amino acid variant thereof. In amino acid variants, one or more R amino acids sometimes are independently substituted with another basic amino acid; the S sometimes is substituted with a hydrophobic amino acid, another neutral hydrophilic amino acid, or an acidic amino acid; the W sometimes is substituted with another hydrophobic amino acid; the D sometimes is substituted with a hydrophobic amino acid or another acidic amino acid; the V sometimes is substituted with another hydrophobic amino acid; the A sometimes is substituted with another hydrophobic amino acid or an acidic amino acid; the N sometimes is substituted with a hydrophobic amino acid, another neutral hydrophilic amino acid, or an acidic amino acid; the F sometimes is substituted with another hydrophobic amino acid; the M sometimes is substituted with another hydrophobic amino acid or an acidic amino acid; the S sometimes is substituted with a hydrophobic amino acid or an acidic amino acid; the S sometimes is substituted with an acidic amino acid; the S sometimes is substituted with a hydrophobic amino acid; the D sometimes is substituted with a hydrophobic amino acid; the D sometimes is substituted with another acidic amino acid; the A sometimes is substituted with another hydrophobic amino acid; the A sometimes is substituted with an acidic amino acid; the N sometimes is substituted with a hydrophobic amino acid or an acidic amino acid; the N sometimes is substituted with an acidic amino acid; the N sometimes is substituted with a hydrophobic amino acid; the M sometimes is substituted with an acidic amino acid; the M sometimes is substituted with a hydrophobic amino acid; two or more or three or more of the S, D, A, N and M sometimes are independently substituted with hydrophobic amino acids; the S, D, A, N and M sometimes are independently substituted with hydrophobic amino acids; the hydrophobic amino acids sometimes are independently selected from alanine and leucine; the hydrophobic amino acids sometimes are alanine; the hydrophobic amino acids sometimes are leucine; or combinations of the foregoing. In some embodiments, the peptide moiety consists essentially of or consists of the amino acid sequence RSRWRDVARNFMR. In some embodiments, the A in the peptide moiety is not substituted by cysteine. In some embodiments, the amino acid sequence does not include two, three, four, or five or more consecutive basic amino acids. In some embodiments, the amino acid sequence does not include a cysteine. In certain embodiments, the amino acid sequence does not include the following “R-Z” sequences: -Z-R-R-Z-Z-R- ; -Z-R-R-Z-R-R-Z; Z-Z--R-R-Z-R-R-Z-; -R-Z-Z-R-R-R-Z-R-R-Z-; -R-Z-Z-R-R-Z-R-R-Z-; -R-R-R-Z-Z-R-R-Z-; where Z is a hydrophobic amino acid and R is a basic or neutral hydrophilic amino acid. In related embodiments, the amino acid sequence of the peptide moiety may include one of the “R-Z” sequences in the preceding sentence, where the N-terminal or C-terminal amino acid in one of the “R-Z” sequences is the terminal amino acid in the peptide moiety amino acid sequence. The peptide moiety often does not form a helix-turn-helix structure, and sometimes does not substantially form a helical structure in an aqueous solution or in a pharmaceutical formulation. In some embodiments, all of the amino acids in the peptide moiety are L-isomer amino acids; all of the amino acids in the peptide moiety are D-isomer amino acids; or the peptide moiety is a mixture of L-isomer and D-isomer amino acids. The composition sometimes comprises a lipophilic molecule linked to the peptide moiety.

The peptide moiety includes an N-terminal moiety (Nₜₑᵣₘ-) or C-terminal moiety (-Cₜₑᵣₘ) already part of the terminus of the peptide as synthesized or produced, or is selected from any known group that can be linked to the terminus of a peptide and does not reduce antimicrobial activity to undetectable levels. The peptide sometimes is linked to a lipophilic molecule, directly or via a linker, where the lipophilic molecule has a hydrophobic character and often increases the overall hydrophobicity of the peptide. Without being bound by theory, the lipophilic molecule is expected to localize (e.g., accumulate) the peptide in a skin substructure or component (e.g., sebum and/or sebaceous gland). A lipophilic molecule sometimes is an N-terminal moiety or a C-terminal moiety, and sometimes is linked to a side chain in an amino acid within the peptide. The lipophilic molecule sometimes has a log p value (described below) of +1 to +6 and sometimes a log p value of +3 to +4.5, where log p values are a measure of hydrophobicity. The lipophilic molecule can be any molecule having a hydrophobic character that can be linked to a peptide, including but not limited to an acyl moiety, a fatty acid moiety, or a lauryl moiety, for example. The lipophilic moiety, such as an acyl moiety, sometimes is linked to the peptide directly by an amide linkage, and sometimes is linked to the peptide via a linker moiety.

In some embodiments, a composition comprises a peptide, which sometimes is referred to as a “peptide moiety” herein, that “consists of” or “consists essentially of” a particular amino acid sequence. Where a peptide “consists of” or “consists essentially of” a particular amino acid sequence, the peptide may include an amino moiety (e.g., NH₂— or NH₃⁺— moiety) or acetyl moiety at the N-terminus, and an amide moiety or a carboxyl moiety (e.g., —COO— or —COOH moiety) at the C-terminus. A composition comprising a peptide may include other molecules appended to the peptide, such as a lipophilic, acyl and/or fatty acid molecule, for example, appended to the N-terminus or C-terminus of the peptide. Where a peptide “consists essentially of” a particular amino acid sequence, the peptide may (1) consist of that sequence or (2) consist of a sequence that includes (a) one, two or three amino acid substitutions to the specified sequence (e.g., conservative amino acid substitutions (described hereafter)) and/or (b) one, two or three amino acid additions or deletions (i) at the N-terminus, (ii) at the C-terminus, (iii) at the N-terminus and C-terminus, or (iv) within the sequence, so long as the peptide moiety retains significant antimicrobial activity, such as an antimicrobial activity of 64 micrograms/milliliter or better in an assay described in Example 9. In embodiments where the peptide “consists essentially of” the amino acid sequence RSRWRDVARNFMR (e.g., L and/or D amino acids) and includes only one additional amino acid appended at the N-terminus and only one additional amino acid appended to the C-terminus, the amino acid appended at the N-terminus is not glycine and the amino acid appended at the C-terminus is not arginine; thus, any one of the other twenty naturally occurring amino acids (e.g., D or L isomers), or derivatives thereof, may be present as the one additional amino acid at each terminus in such embodiments (e.g., alanine, valine, leucine, isoleucine, phenylalanine, tyrosine, tryptophan, histidine, lysine, arginine, serine, threonine, cysteine, methionine, aspartate, glutamate, asparagine, glutamine, proline (D or L isoforms) or derivatives thereof may be appended as the one appended amino acid at the N-terminus, and alanine, valine, leucine, isoleucine, phenylalanine, tyrosine, tryptophan, histidine, lysine, glycine, serine, threonine, cysteine, methionine, aspartate, glutamate, asparagine, glutamine, proline (D or L isoforms) or derivatives thereof may be appended as the one appended amino acid at the C-terminus).

Peptides consisting of amino acids alone and peptides in combination with a lipophilic moiety or other modification collectively are referred to herein as “peptide compositions.” Specific peptide composition embodiments are disclosed in Table 3 hereafter. Also provided is a pharmaceutical composition comprising a peptide composition described herein with a pharmaceutically acceptable carrier. In certain embodiments, the pharmaceutical composition comprises one or more gel components useful for topical application to human skin, and sometimes the composition is a cream, ointment, lotion, cosmetic or wash, and sometimes is in a medicated pad, patch, strip or bandage (e.g., a peptide composition described herein is applied to or impregnated in a pad, patch, strip or bandage before or after the product is purchased by a consumer).

Featured also are methods for using a peptide composition or pharmaceutical composition described herein. One embodiment is a method for reducing a microbe population in a system, which comprises administering a composition to the system in an amount that reduces the microbe population, where the composition comprises a peptide composition disclosed herein. In certain embodiments, the microbe is selected from the group consisting of Salmonella, Staphylococcus, Propionibacterium, Escherichia, Pseudomonas, Pityrosporum, Candida and Trichophyton, and sometimes is selected from the group consisting of Salmonella dublin, Staphylococcus aureus, Propionibacterium acnes, Escherichia coli, Pseudomonas aeruginosa, Staphylococcus epidermidis, Pityrosporum ovate, Candida albicans and Trichophyton rubrum. In some embodiments, the system is skin, often human skin, and sometimes the composition is delivered by topical administration to the human skin.

Also featured is a method for reducing inflammation in a human tissue, which comprises administering a composition to the human tissue in an amount that reduces the inflammation, where the composition comprises a peptide composition disclosed herein. The tissue often is human skin and the composition often is delivered by topical administration to the human skin.

Featured also is a method for treating a skin condition such as acne, which comprises administering a composition to human skin in an amount that treats the skin condition, where the composition comprises a peptide composition disclosed herein. In certain embodiments, the skin condition is acne vulgaris and in other embodiments, the skin condition is acne conglobate. The composition often is delivered by topical administration to the human skin.

Also featured is a method for selectively delivering an antimicrobial composition to one or more skin substructures or components (e.g., one or more of those described above), which comprises administering a composition to the skin in an amount that selectively delivers the composition to the skin substructure or component, where the composition comprises an antimicrobial peptide linked to a lipophilic moiety. The composition often is delivered by topical administration to the skin, the skin sometimes is not integrated with a subject (i.e., the skin is removed from the subject), the skin often is integrated with a subject (i.e., the skin is not removed from the subject), and the skin often is human skin.

Featured also is an apparatus useful for mounting and contacting a skin sample with biological reagents. A skin sample from a subject often rounds after excision, making it difficult to manipulate the sample. The apparatus described herein, an embodiment of which is illustrated in FIGS. 1A-1D and described in further detail hereafter, overcomes this technical difficulty by conforming the skin sample to a flat surface. In certain embodiments, the skin sample is sandwiched between the plate shown in FIG. 1A and the plate shown in FIG. 1B in the assembly illustrated in FIG. 1C. Biological reagents, such as peptide compositions described herein and microbial isolates, then are contacted with the skin in the apparatus. The apparatus therefore is useful for determining whether a peptide composition exerts a biological function on and/or in skin (e.g., reducing a microbial population on and/or in skin mounted in the apparatus).

### BRIEF DESCRIPTION OF THE DRAWINGS

FIGS. 1A-1D show multichannel apparatus embodiments useful for mounting a skin sample and determining microbial populations on and/or in the sample. FIG. 1A depicts the top plate, FIG. 1B depicts the bottom plate, and FIG. 1C depicts an assembled top and bottom plate in an apparatus. FIG. 1D shows a side view of the apparatus as it is being assembled with a skin sample mounted between the two plates, and lists certain specifications for apparatus embodiments. FIG. 1A shows the cylindrical shape of channels in the top plate for the front row of channels, for both sample access channels and channels used for mounting fasteners, and FIG. 1B shows the partially cylindrical and partially conical shape of wells in the first row of the bottom plate.

### DETAILED DESCRIPTION

Described herein are peptide compositions having antimicrobial activity and a variety of uses. For example, the peptide compositions are useful for reducing microbial populations on and/or in skin, reducing inflammation in skin, inhibiting a bacterial lipase on and/or in skin, targeting skin substructures and/or components (e.g., one or more of those described above), and treating a skin condition such as acne (e.g., acne vulgaris, acne conglobate). Peptide compositions, pharmaceutical compositions and uses thereof are described in greater detail hereafter.

Peptide Compositions Having Antimicrobial Activity

A peptide composition comprises a peptide moiety having antimicrobial activity. In some embodiments, the peptide composition consists of one or more antimicrobial peptides described hereafter, and in other embodiments the peptide composition consists of an antimicrobial peptide moiety linked to a lipophilic molecule. Peptide compositions can be formulated with a pharmaceutically acceptable carrier in a pharmaceutical composition described hereafter.

A peptide moiety in a composition can include any amino acid sequence that imparts an antimicrobial activity. In some embodiments, the peptide moiety comprises or consists of a native subsequence of an antimicrobial protein, or a variant thereof. Examples of antimicrobial proteins and peptides are known (see e.g., Marshall & Arenas, Electronic J. Biotechnology ISSN: 0717-3458, vol 6 (2003) and documents cited therein), including but not limited to neuropeptides (e.g., peptide B and enkelytin); aspartic acid rich proteins and peptides (e.g., H-GDDDDDD-OH, dermcidin, maximin H5), aromatic dipeptides (e.g., N-beta-alanyl-5-S-glutathionyl-3,4-dihydroxyphenylalanine, p-hydroxycinnamaldehyde); peptides from oxygen binding proteins (e.g., hemocyanin, hemoglobin, lactoferrin); linear alpha-helix peptides (e.g., cecropins, clavanin, styelin, buforins, pleurocidin, moronecidin), proline rich peptides and proteins (e.g., drosocin, metchnikowins, pyrrhocoricin, metalnikowin); glycine rich peptides and proteins (e.g., diptericins, attacins, shepherin I and shepherin II, Ac-AMP1, Ac-AMP2); histidine rich peptides and proteins (e.g., histatin, shepherin I and shepherin II); tyrosine rich peptides and proteins (e.g., indolicidin, tritrpticin, lactoferrin B, lfcinB4-9 ); peptides and proteins having a single disulfide bridge (e.g., thanatin, brevinins, lanthionins); peptides and proteins having two disulfide bridges (e.g., tachyplesin II, androctonin, protegrin I); peptides and proteins having three disulfide bridges (e.g., alpha defensins, beta defensins, defensis, penaeidins); peptides and proteins having more than three disulfide bridges (e.g., tachycitin, drosomycin, gambicin, heliomicin); and plant derived peptides and proteins (e.g., defensin protein WT1, alfAFP defensin, So-D1-7, DmAMP1). In specific embodiments, the peptide moiety in the composition comprises or consists of a subsequence of a granulysin having antimicrobial activity, often a human granulysin, or a variant thereof having antimicrobial activity. Examples of human granulysin sequences are known (e.g., U.S. Pat. No. 6,485,928) and are disclosed hereafter: SEQ ID NO: 19 listed hereafter is a 9 kD form and proteolysis product of P519; SEQ ID NO: 20 listed hereafter is referred to as P519; SEQ ID NO: 21 listed hereafter is referred to as P520; SEQ ID NO: 22 listed hereafter is referred to as P522; amino acids 16-145 of SEQ ID NO: 21 is a mature form of P520 with a signal sequence cleaved; and amino acids 16-172 of SEQ ID NO: 22 is a mature form of P522 with a cleaved signal sequence.

| Gly Arg Asp Tyr Arg Thr Cys Leu Thr Ile Val Gln Lys Leu Lys Lys | SEQ ID NO: 19 |
|---|---|
| 1 5 10 15 | |
| Met Val Asp Lys Pro Thr Gln Arg Ser Val Ser Asn Ala Ala Thr Arg | |
| 20 25 30 | |
| Val Cys Arg Thr Gly Arg Ser Arg Trp Arg Asp Val Cys Arg Asn Phe | |
| 35 40 45 | |
| Met Arg Arg Tyr Gln Ser Arg Val Ile Gln Gly Leu Val Ala Gly Glu | |
| 50 55 60 | |
| Thr Ala Gln Gln Ile Cys Glu Asp Leu Arg | |
| 65 70 | |
| Met Glu Gly Leu Val Phe Ser Arg Leu Ser Pro Glu Tyr Tyr Asp Pro | SEQ ID NO: 20 |
| 1 5 10 15 | |
| Ala Arg Ala His Leu Arg Asp Gly Glu Lys Ser Cys Pro Cys Gly Gln | |
| 20 25 30 | |
| Glu Gly Pro Gln Gly Asp Leu Leu Thr Lys Thr Gln Glu Leu Gly Arg | |
| 35 40 45 | |
| Asp Tyr Arg Thr Cys Leu Thr Ile Val Gln Lys Leu Lys Lys Met Val | |
| 50 55 60 | |
| Asp Lys Pro Thr Gln Arg Ser Val Ser Asn Ala Ala Thr Arg Val Cys | |
| 65 70 75 80 | |
| Arg Thr Gly Arg Ser Arg Trp Arg Asp Val Cys Arg Asn Phe Met Arg | |
| 85 90 95 | |
| Arg Tyr Gln Ser Arg Val Ile Gln Gly Leu Val Ala Gly Glu Thr Ala | |
| 100 105 110 | |
| Gln Gln Ile Cys Glu Asp Leu Arg Leu Cys Ile Pro Ser Thr Gly Pro | |
| 115 120 125 | |
| Leu | |
| Met Ala Thr Trp Ala Leu Leu Leu Leu Ala Ala Met Leu Leu Gly Asn | SEQ ID NO: 21 |
| 1 5 10 15 | |
| Pro Gly Leu Val Phe Ser Arg Leu Ser Pro Glu Tyr Tyr Asp Leu Ala | |
| 20 25 30 | |
| Arg Ala His Leu Arg Asp Glu Glu Lys Ser Cys Pro Cys Leu Ala Gln | |
| 35 40 45 | |
| Glu Gly Pro Gln Gly Asp Leu Leu Thr Lys Thr Gln Glu Leu Gly Arg | |
| 50 55 60 | |
| Asp Tyr Arg Thr Cys Leu Thr Ile Val Gln Lys Leu Lys Lys Met Val | |
| 65 70 75 80 | |
| Asp Lys Pro Thr Gln Arg Ser Val Ser Asn Ala Ala Thr Arg Val Cys | |
| 85 90 95 | |
| Arg Thr Gly Arg Ser Arg Trp Arg Asp Val Cys Arg Asn Phe Met Arg | |
| 100 105 110 | |
| Arg Tyr Gln Ser Arg Val Thr Gln Gly Leu Val Ala Gly Glu Thr Ala | |
| 115 120 125 | |
| Gln Gln Ile Cys Glu Asp Leu Arg Leu Cys Ile Pro Ser Thr Gly Pro | |
| 130 135 140 | |
| Leu | |
| Met Ala Thr Trp Ala Leu Leu Leu Leu Ala Ala Met Leu Leu Gly Asn | SEQ ID NO: 22 |
| 1 5 10 15 | |
| Pro Gly Leu Glu Val Ser Val Ser Pro Lys Gly Lys Asn Thr Ser Gly | |
| 20 25 30 | |
| Arg Glu Ser Gly Phe Gly Trp Ala Ile Trp Met Glu Gly Leu Val Phe | |
| 35 40 45 | |
| Ser Arg Leu Ser Pro Glu Tyr Tyr Asp Leu Ala Arg Ala His Leu Arg | |
| 50 55 60 | |
| Asp Glu Glu Lys Ser Cys Pro Cys Leu Ala Gln Glu Gly Pro Gln Gly | |
| 65 70 75 80 | |
| Asp Leu Leu Thr Lys Thr Gln Glu Leu Gly Arg Asp Tyr Arg Thr Cys | |
| 85 90 95 | |
| Leu Thr Ile Val Gln Lys Leu Lys Lys Met Val Asp Lys Pro Thr Gln | |
| 100 105 110 | |
| Arg Ser Val Ser Asn Ala Ala Thr Arg Val Cys Arg Thr Gly Arg Ser | |
| 115 120 125 | |
| Arg Trp Arg Asp Val Cys Arg Asn Phe Met Arg Arg Tyr Gln Ser Arg | |
| 130 135 140 | |
| Val Ile Gln Gly Leu Val Ala Gly Glu Thr Ala Gln Gln Ile Cys Glu | |
| 145 150 155 160 | |
| Asp Leu Arg Leu Cys Ile Pro Ser Thr Gly Pro Leu | |
| 165 170 | |

In some embodiments, the peptide moiety comprises or consists of a full-length amino acid sequence of a native antimicrobial protein, or a variant thereof, and in other embodiments, the peptide moiety comprises or consists of a native amino acid subsequence of the antimicrobial protein having antimicrobial activity, or a variant thereof having antimicrobial activity. Any subsequence length can be screened using methods for determining antimicrobial activity, examples of which are described herein. In certain embodiments, the peptides screened for antimicrobial activity comprise or consist of an amino acid subsequence from a native antimicrobial protein that is 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 amino acids in length, and certain embodiments, the peptide composition amino acid sequence comprises or consists of a subsequence from SEQ ID NO: 19, or a variant sequence thereof. In specific embodiments, the peptide composition amino acid sequence comprises or consists of amino acids 31-50 or 38-50 in SEQ ID NO: 19, or a variant sequence thereof.

A peptide moiety in a composition is synthesized or prepared by known techniques. Peptides can be synthesized on a solid support or in solution (e.g., see Creighton, 1983, Proteins: Structures and Molecular Principles, W. H. Freeman and Co., N.Y.; Merrifield, R. B. J. Am. Chem. Soc. 85, 2149-2153, 1963; W. C. Chan and P. D. White, Fmoc Solid Phase Peptide Synthesis: a Practical Approach, Oxford University Pres, Oxford, 2000; Bodanszky, M. Principles of Peptide Synthesis, Second Ed., Springer, New York, 1993). Longer peptides may be generated using recombinant DNA techniques (see e.g., Sambrook, et al., 1989, Molecular Cloning, A Laboratory Manual, Vols. 1-3, Cold Spring Harbor Press, N.Y.).

In certain embodiments, a peptide moiety in a composition does not comprise or consist of a native amino acid sequence or subsequence of an antimicrobial protein, but comprises or consists of a variant sequence or subsequence having antimicrobial activity. A variant peptide moiety sometimes differs by one or more amino acid substitutions, insertions or deletions, such as 1, 2, 3, 4, 5, 6, 7, 8, 9 or 10 amino acid substitutions, insertions or deletions from the native sequence or subsequence, and sometimes is substantially identical to the native peptide sequence or subsequence.

The term “substantially identical” as used herein refers to peptides sharing one or more identical amino acid sequences. Included is an amino acid sequence that is 55% or more, 60% or more, 65% or more, 70% or more, 75% or more, 80% or more, 85% or more, 90% or more, or 95% or more (each often within a 1%, 2%, 3% or 4% variability) identical to another amino acid sequence. One test for determining whether two peptides are substantially identical is to determine the percent of identical amino acid sequences shared between the peptides.

Calculations of sequence identity can be performed as follows. Sequences are aligned for optimal comparison purposes (e.g. gaps can be introduced in one or both of a first and a second amino acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The amino acids at corresponding peptide positions then are compared among the two sequences. When a position in the first sequence is occupied by the same amino acid as the corresponding position in the second sequence, the amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining if two amino acid sequences are substantially identical is to assess whether they are encoded by polynucleotide sequences that will hybridize to one another under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC,0.1% SDS at 65° C. More often, stringency conditions are 0.5M sodium phosphate, 7% SDS at 65° C., followed by one or more washes at 0.2×SSC, 1% SDS at 65° C.

An amino acid sequence can be used as a “query sequence” to perform a search against public databases to identify other family members or related sequences, for example. Such searches can be performed using the NBLAST and XBLAST programs (version 2.0) of Altschul et al., J. Mol. Biol. 215: 403-10 (1990). BLAST amino acid searches can be performed with the XBLAST program, score=50, wordlength=3. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g. XBLAST and NBLAST) can be used (see http address www.ncbi.nlm.nih.gov).

A variant peptide moiety can depart from a native amino acid sequence in different manners. Amino acid substitutions may be made on the basis of similarity in polarity, charge, solubility, hydrophobicity, hydrophilicity, helix-forming properties and/or amphipathic properties and the resulting variants are screened for antimicrobial activity. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine. Conservative substitutions may be made, for example, according to Table 2. Amino acids in the same block in the second column and in the same line in the third column may be substituted for one another other in a conservative substitution. Certain conservative substitutions are substituting an amino acid in one row of the third column corresponding to a block in the second column with an amino acid from another row of the third column within the same block in the second column.

TABLE 2

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In certain embodiments homologous substitution may occur, which is a substitution or replacement of like amino acids, such as basic for basic (polar-charged), acidic for acidic (polar charged), polar for polar amino acids, neutral hydrophilic for neutral hydrophilic (polar uncharged) and hydrophobic for hydrophobic, for example. Non-homologous substitutions can be introduced to a native sequence, such as from one class of residue to another (e.g., a non-hydrophobic to a hydrophobic amino acid), or substituting a naturally occurring amino acid with an unnatural amino acid or non-classical amino acid replacements such as ornithine, diaminobutyric acid, norleucine, pyrylalanine, thienylalanine, naphthylalanine and phenylglycine. Other examples of non-naturally occurring amino acids and non-classical amino acid replacements are alpha and alpha-disubstituted amino acids, N-alkyl amino acids, lactic acid*, halide derivatives of natural amino acids such as trifluorotyrosine*, p-X-phenylalanine (where X is a halide such as F, Cl, Br, or I)*, allylglycine*, 7-aminoheptanoic acid*, methionine sulfone*, norleucine*, norvaline*, p-nitrophenylalanine*, hydroxyproline#, thioproline*, methyl derivatives of phenylalanine (Phe) such as 4-methyl-Phe*, pentamethyl-Phe*, Phe (4-amino)#, Tyr (methyl)*, Phe (4-isopropyl)*, Tic (1,2,3,4-tetrahydroisoquinoline-3-carboxyl acid)*, diaminopropionic acid, Phe (4-benzyl)*, 4-aminobutyric acid (gamma-Abu)*, 2-aminobutyric acid (alpha-Abu)*, 6-aminohexanoic acid (epsilon-Ahx)*, 2-aminoisobutyric acid (Aib)*, 3-aminopropionic acid*, norvaline*, hydroxyproline, sarcosine, citrulline, homocitrulline, cysteic acid, t-butylglycine*, t-butylalanine*, phenylglycine*, cyclohexylalanine*, fluoroamino acids, designer amino acids such as beta-methyl amino acids, and the like. The notation * indicates a derivative having hydrophobic characteristics and # indicates a derivative having hydrophilic characteristics. Amino acid substitutions sometimes are selected to enhance the hydrophobicity of the variant peptide, the amphipathic nature of a variant peptide, and to enhance or decrease the probability that a variant peptide forms an alpha-helical structure or substructure.

Variant amino acid sequences sometimes include suitable spacer groups inserted between any two amino acid residues of the sequence, such as alkyl groups (e.g., methyl, ethyl or propyl groups) or amino acid spacers (e.g., glycine or beta-alanine). Peptide moieties sometimes comprise or consist of peptoids. The term “peptoids” refers to variant amino acid structures where the alpha-carbon substituent group is linked to the backbone nitrogen atom rather than the alpha-carbon. Processes for preparing peptides in peptoid form are known (e.g., Simon et al., PNAS (1992) 89(20), 9367-9371 and Horwell, Trends Biotechnol. (1995) 13(4), 132-134).

As disclosed above, the peptide composition often includes an amino acid sequence that conforms with a sequence motif pattern in Table 1. In Table 1, B₁, B₂, B₃, B₄ and B₅ are independently selected from basic amino acids, Z₁, Z₂ and Z₃ are independently selected from hydrophobic amino acids, and X₁, X₂, X₃, X₄ and X₅ are independently selected from any amino acid.

Basic amino acids include, but are not limited to, arginine, homoarginine and all other homologs of arginine, lysine and its homologs (such as ornithine), histidine, diaminobutyric acid, citrulline and p-aminophenylalanine. In some embodiments B₁, B₂, B₃, B₄ and B₅ are identical (e.g., all are arginine or homoarginine), and often one or more are different basic amino acids (e.g., two are arginine and three are homoarginine). B₁, B₂, B₃, B₄ and B₅ sometimes are independently selected from the group consisting of arginine, homoarginine and all other homologs of arginine, lysine and its homologs (such as ornithine), histidine and diaminobutyric acid or a subset thereof; sometimes are independently selected from the group consisting of arginine, homoarginine and all other homologs of arginine, lysine and its homologs (such as ornithine), or a subset thereof; sometimes are independently selected from the group consisting of arginine, homoarginine, lysine and ornithine, or a subset thereof; sometimes are independently selected from the group consisting of arginine and homoarginine or a subset thereof; sometimes are independently selected from the group consisting of arginine and lysine, sometimes all are lysine, and sometimes all are arginine.

Hydrophobic amino acids include, but are not limited to, alanine, naphthylalanine, biphenylalanine, valine, leucine, isoleucine, phenylalanine, homophenylalanine, tryptophan, methionine, cyclohexylalanine, aminoisobutyric acid, norvaline, norleucine, tert-leucine, tetrahydroisoquinoline carboxylic acid, pipecolic acid, phenylglycine, cyclohexylglycine, dehydroleucine, 2,2-diethylglycine, 1-amino-1-cyclopentane carboxylic acid, 1-amino-1-cyclohexane carboxylic acid, aminobenzoic acid, aminonaphthyl carboxylic acid, 7-aminobutyric acid, difluorophenylalanine, fluorophenylalanine, nipecotic acid, aminobutyric acid, thienylalanine and t-butyl-glycine. Z₁, Z₂ and Z₃ sometimes are independently selected from the group consisting of alanine, naphthylalanine, biphenylalanine, valine, leucine, isoleucine, phenylalanine, homophenylalanine, tryptophan, methionine, cyclohexylalanine, aminoisobutyric acid, norvaline, norleucine, tert-leucine, phenylglycine, cyclohexylglycine, 2,2-diethylglycine, 1-amino-1-cyclopentane carboxylic acid, 1-amino-1-cyclohexane carboxylic acid, aminobenzoic acid, aminonaphthyl carboxylic acid, 7-aminobutyric acid, aminobutyric acid and t-butyl-glycine, or a subset thereof; sometimes are independently selected from the group consisting of alanine, naphthylalanine, biphenylalanine, valine, leucine, isoleucine, phenylalanine, homophenylalanine, tryptophan, methionine, cyclohexylalanine, aminoisobutyric acid, norvaline, norleucine, tert-leucine, phenylglycine, cyclohexylglycine, 2,2-diethylglycine and t-butyl-glycine or a subset thereof; sometimes are independently selected from the group consisting of alanine, naphthylalanine, biphenylalanine, valine, leucine, isoleucine, phenylalanine, homophenylalanine, tryptophan, norvaline, norleucine and tert-leucine or a subset thereof; sometimes are independently selected from the group consisting of alanine, naphthylalanine, valine, leucine, phenylalanine and tryptophan or a subset thereof; sometimes are independently selected from the group consisting of an amino acid with an aliphatic side chain (e.g., alanine, valine, leucine and isoleucine) and tryptophan; sometimes are independently selected from the group consisting of tryptophan, leucine, valine and napthylalanine; sometimes are independently selected from the group consisting of alanine, leucine and valine; sometimes are independently selected from the group consisting of leucine and alanine; sometimes are alanine; and sometimes are leucine.

In certain embodiments, X₁, X₂, X₃, X₄ and X₅ sometimes are independently selected from the group consisting of any amino acid in D-form or L-form, natural or unnatural, and homologs of alpha amino acids such as beta²-, beta³-, and beta^{2,3} amino acids and gamma amino acids; sometimes are selected from the group consisting of any amino acid in D-form or L-form, natural or unnatural, and beta³ amino acids; sometimes are selected from the group consisting of any amino acid, natural or unatural, in D- or L-form; sometimes are selected from the group consisting of any of the 20 naturally occurring alpha amino acids, ornithine and homoarginine, in D- or L-form; sometimes are independently selected from the group consisting of alanine, leucine, valine, tryptophan, phenylalanine, serine, glutamic acid, aspartic acid, lysine, ornithine, arginine and homoarginine in D- or L-form; sometimes are independently selected from the group consisting of alanine, leucine, arginine and lysine in D- or L-form; sometimes are independently selected from hydrophobic amino acids or a subset thereof (e.g., leucine, valine, alanine); sometimes are independently selected from basic amino acids or a subset thereof (e.g., lysine and arginine); sometimes are independently selected from acidic amino acids, including but not limited to aspartate and glutamate; and sometimes are independently selected from hydrophilic neutral amino acids, including but not limited to serine, threonine, methionine, cysteine, asparagine, glutamine, proline and glycine or a subset thereof (e.g., threonine, serine, glutamine and asparagine). X₁ sometimes is selected from the group consisting of serine, alanine, and leucine in D- or L-form; X₂ sometimes is selected from a hydrophilic amino acid, including but not limited to, serine, threonine, aspartate, glutamate, asparagine and glutamine, an acidic amino acid, including but not limited to aspartate and glutamate, each in D- or L-form; and X₃ sometimes is selected from the group consisting of alanine, leucine, isoleucine, phenylalanine, tryptophan, arginine, or lysine, each in D- or L-form.

In some embodiments, X₃ is Y₁ and X₅ is Y₂, where Y₁ and Y₂ are independently selected from the group consisting of alanine, leucine, tryptophan and methionine in D- or L-form. In certain embodiments, Y₁ is selected from the group consisting of alanine, leucine and tryptophan and in certain embodiments Y₂ is selected from the group consisting of alanine, leucine and methionine. In some embodiments X₃ and X₅ are independently selected from the group consisting of an amino acid with an aliphatic side chain (e.g., alanine, valine, leucine and isoleucine) and tryptophan, an in certain embodiments Z₁, Z₂, Z₃, X₃ and X₅ are independently selected from the group consisting of an amino acid with an aliphatic side chain (e.g., alanine, valine, leucine and isoleucine) and tryptophan.

For the motifs of Table 1, any combinations of the foregoing selections for each amino acid position are included herein. For example, in certain embodiments, the peptide composition conforms to the motif Nₜₑᵣₘ-B₁—X₁—B₂-Z₁-B₃—X₂-Z₂-X₃—B₄—X₄-Z₃-X₅—B₅—NH₂, where Nₜₑᵣₘ- is an acyl group (e.g., a lauryl moiety); B₁, B₂, B₃, B₄, and B₅ are independently selected from basic D-amino acids; Z, Z₂, Z₃, Z₄, and Z₅ are independently selected from D-amino acids with aliphatic side chains, Phe or Trp; and X₁, X₂, and X₃ are independently selected from D-amino acids. In some embodiments, the peptide composition conforms to the motif Nₜₑᵣₘ—B₁—X₁—B₂-Z₁-B₃—X₂-Z₂-X₃—B₄—X₄-Z₃-X₅—B₅—NH₂, wherein Nₜₑᵣₘ— is a free amine or acylated terminus; B₁, B₂, B₃, B₄, and B₅ are independently selected from basic D-amino acids; Z₁, Z₂, Z₃, Z₄, and Z₅ are independently selected from hydrophobic D-amino acids; and X₁, X₂, and X₃ are independently selected from D-amino acids. In certain embodiments, the peptide composition conforms to the motif Nₜₑᵣₘ—B₁—X₁—B₂-Z₁-B₃—X₂-Z₂-Y₁—B₄—X₄-Z₃-Y₂—B₅—NH₂, where Nₜₑᵣₘ— is an acyl group (e.g., a lauryl moiety); B₁, B₂, B₃, B₄, and B₅ are independently selected from the group consisting of arginine, homoarginine, lysine, and ornithine; Z₁, Z₂ and Z₃ are independently selected from the group consisting of tryptophan, leucine, valine, and naphthylalanine; Y₁ is selected from the group consisting of alanine, leucine and tryptophan; Y₂ is selected from the group consisting of alanine, leucine and methionine; and X₁, X₂, and X₃ are independently selected from D-amino acids. In some embodiments, the peptide composition conforms to the motif Nₜₑᵣₘ—B₁—X₁—B₂-Z₁-B₃—X₂-Z₂-Y₁—B₄—X₄-Z₃-Y₂—B₅—NH₂, where Nₜₑᵣₘ— is an acyl group (e.g., a lauryl moiety); B₁, B₂, B₃, B₄, and B₅ are independently selected from the group consisting of arginine, homoarginine, lysine, and ornithine; Z₁, Z₂ and Z₃ are independently selected from the group consisting of tryptophan, leucine, valine, and naphthylalanine; Y₁ is selected from the group consisting of alanine, leucine and tryptophan; Y₂ is selected from the group consisting of alanine, leucine and methionine; X₁ is independently selected from the group consisting of serine, alanine and leucine; X₂, is independently selected from the group consisting of aspartate, glutamate, alanine and leucine; and X₃ is independently selected from the group consisting of alanine, leucine, isoleucine, phenylalanine, tryptophan, arginine, or lysine. In certain embodiments, the peptide composition conforms to the motif Nₜₑᵣₘ—B₁—X₁-B₂-Z₁-B₃—X₂-Z₂-Y₁—B₄—X₄-Z₃-Y₂—B₅—NH₂, where Nₜₑᵣₘ— is an acyl group (e.g., a lauryl moiety); B₁, B₂, B₃, B₄, and B₅ are independently selected from the group consisting of arginine, homoarginine, lysine, and ornithine; Z₁, Z₂ and Z₃ are independently selected from the group consisting of tryptophan, leucine, valine, and naphthylalanine; Y₁ is selected from the group consisting of alanine, leucine and tryptophan; Y₂ is selected from the group consisting of alanine, leucine and methionine; X₁ is independently selected from the group consisting of serine, alanine and leucine; X₂, is independently selected from the group consisting of aspartate, gluatamate, serine, threonine, asparagine and glutamine; and X₃ is independently selected from the group consisting of alanine, leucine, isoleucine, phenylalanine, tryptophan, arginine, or lysine.

In certain embodiments, the peptide is linked to another molecule, such as another peptide for example, that enhances cell penetrance. A peptide that enhances cell penetrance is referred to herein as a “protein transduction domain (PTD)” peptide or “transduction peptide.” A cell penetrance enhancement sometimes is identified when a greater amount of the peptide composition is translocated across a cell membrane in a certain time frame when conjugated to a PTD as compared to peptide composition not conjugated to a PTD. A PTD can be conjugated to a peptide composition using known methods (e.g., U.S. patent application Ser. No. 60/524,152 filed Nov. 20, 2003). PTD peptides are known, and include amino acid subsequences from HIV-tat (e.g., U.S. Pat. No. 6,316,003), sequences from a phage display library (e.g., U.S. 20030104622) and sequences rich in amino acids having positively charged side chains (e.g., sequences having amino acids with guanidino-, amidino- and amino-containing side chains, such as RRQRRTSKLMKR, polyornithine (e.g., (ornithine)₈) and polylysine (e.g., (lysine)₈); see also e.g., U.S. Pat. No. 6,593,292). The PTD peptide sometimes is branched, and in an embodiment, the branched PTD is X₄K(Ahx-RRQRRTSKLMKR)₂, where X₄ is Cys or H₂N-GlyGly.

A peptide moiety in a composition sometimes is synthesized such that one or more of the bonds which link the amino acids are non-peptide bonds. These alternative non-peptide bonds (e.g., imino, ester, hydrazide, semicarbazide, azo, alkene, and cis- or trans-alkene bonds) are formed by known reactions. In some embodiments, a peptide moiety in a composition is synthesized with an altered steric configuration. For example, the D-isomer of one, two or more, or all amino acids in the peptide moiety sometimes is incorporated in a peptide rather than the usual L-isomer.

A variant peptide moiety in a composition sometimes comprises a N-terminal and/or C-terminal modification, i.e., a moiety different than or linked to a N-terminal amino group that is part of the N-terminal amino acid and different than or linked to a C-terminal carboxyl moiety that is part of the C-terminal amino acid. Examples of N-terminal modifications include but are not limited to a hydrophobic or lipophilic moiety (e.g., carbobenzoxyl, dansyl, t-butyloxycarbonyl or another lipophilic moiety described herein); an acetyl moiety; a 9-fluorenylmethoxy-carbonyl (Fmoc) moiety; beta-alanine moiety; or a macromolecular carrier moiety (e.g., a lipid fatty acid conjugate, polyethylene glycol or a carbohydrate). C-terminal modifications include but are not limited to an amido moiety; a hydrophobic or lipophilic moiety, such as a lipophilic moiety described herein; peptide esters (e.g., methyl, ethyl, t-butyl, and other hydrophobic esters); substituted amides (e.g., N-alkyl amides); a free carboxylic acid or carboxylate moiety of native peptides; or a macromolecular carrier group. Such modifications sometimes enhance stability and protease resistance of the peptide moiety in the composition, and sometimes enhances localization to a skin substructure when the peptide composition is administered to a subject.

Specific peptide composition embodiments are listed in the following Table 3.

TABLE 3

| | | |
|---|---|---|
| PEPTIDE | | SEQ ID |
| NUMBER | PEPTIDE SEQUENCE | NO |

| | | |
|---|---|---|
| 1 | Ac-TRVSRTGRSRWRDWSRNFMRAA-NH2 | 23 |
| 2 | Ac-TRVSRTGRSRWRDWSRNFMRAARRRRRRRR-NH2 | 24 |
| 3 | Ac-TRVSRTGRSRWRDWSRNFMRAAOOOOOOOO-NH2 | 25 |
| 4 | Ac-TRVSRTGRSRWRDWSRNFMRAAKKKKKKKK-NH2 | 26 |
| 5 | Ac-TRVSRTGRSRWRDWSRNFMRAAKKKK-NH2 | 27 |
| 6 | Ac-TRVSRTGRSRWRDWSRNFMRAAKKKKK-NH2 | 28 |
| 7 | Ac-TRVSRTGRSRWRDWSRNFMRAAKKKKKK-NH2 | 29 |
| 8 | Ac-KKKKAATRVSRTGRSRWRDWSRNFMRAAKKKK-NH2 | 30 |
| 9 | Ac-TRVSRTGRSRWRDWSRNFMRAARRRR-NH2 | 31 |
| 10 | Ac-TRVSRTGRSRWRDWSRNFMRAARRRRR-NH2 | 32 |
| 11 | Ac-TRVSRTGRSRWRDWSRNFMRAARRRRRR-NH2 | 33 |
| 12 | H2N-RRRRAATRVSRTGRSRWRDWSRNFMRAARRRR-NH2 | 34 |
| 13 | Ac-TRVSRTGRSRWRDWSRNFMRRR-NH2 | 35 |
| 14 | Ac-TRVSRTGRSRWRDWSRNWMRRR-NH2 | 36 |
| 15 | Ac-ERVSRTGRSRWRDWSRNFMRRR-NH2 | 37 |
| 16 | Ac-RRVSRTGRSRWRDWSRNFMRRR-NH2 | 38 |
| 17 | Ac-AATRVSRTGRSRWRDWSRNFMR-NH2 | 39 |
| 18 | betaAla-TRVSRTGRSRWRDWSRNFMRAA-NH2 | 40 |
| 19 | Ac-TRVSRTGRSRWRDWSRNFMRLL-NH2 | 41 |
| 20 | Ac-TRVSRTGRSRWRDVSRNFMR-NH2 | 42 |
| 21 | (TRVSRTGRSRWRDWSRNFMRAAZ)2-K Z = aminohexanoyl | 43 |
| 22 | XAAZAAX; X = TRVSRTGRSRWRDWSRNFMR, Z = aminohexanoyl | 44 |
| 23 | Ac-TRVZRTGRSRWRDWDRNFMR-NH2 | 45 |
| | Z = diaminopropionic acid − Z-D15 lactam bridge | |
| 24 | Ac-TRVDRTGRSRWRDWZRNFMR-NH2 | 46 |
| | Z = diaminopropionic acid − D4-Z lactam bridge | |
| 25 | Ac-TRVZRTGRSRWRDVDRNFMR-NH2 | 47 |
| | Z = diaminopropionic acid − Z-D15 lactam bridge | |
| 26 | Ac-TRVDRTGRSRWRDVZRNFMR-NH2 | 48 |
| | Z = diaminopropionic acid − D4-Z lactam bridge | |
| 27 | Ac-RSVSNAATRVSRTGRSRWRDWSRNFMR-NH2 | 49 |
| 28 | Ac-RDYRTSLTIVQKXTRVSRTGRSRWRDWSRNFMR-NH2 | 50 |
| | X = aminohexanoic acid | |
| 29 | Ac-trvsrtgrsrwrdwsrnfmrll-NH2 | 51 |
| 30 | Ac-trvsrtgrsrwrdwsrnfmraa-NH2 | 52 |
| 31 | Ac-trvsrtgrsrwrdwsrnfmraakkkkkkkk-NH2 | 53 |
| 32 | Ac-trvsrtgrsrwrdwsrnfmraaoooooooo-NH2 | 54 |
| 33 | Ac-trvsrtgrsrwrdwsrnfmr-NH2 | 55 |
| 34 | Ac-trvsrtgrsrwrdwsrnfmrkkkkkkkk-NH2 | 56 |
| 35 | Ac-trvsrtgrsrwrdwsrnfmroooooooo-NH2 | 57 |
| 36 | H2N-kkkkkkkkaatrvsrtgrsrwrdwsrnfmr-NH2 | 58 |
| 37 | Ac-TRVARTGRSRWRDWARNFMR-NH2 | 59 |
| 38 | Ac-TRVARTGRSRWRDVARNFMR-NH2 | 60 |
| 39 | Ac-TRVARTGRSRWRDVARNFMRAA-NH2 | 61 |
| 40 | Ac-TRVARTGRSRWRDVARNFMRAARRRRRRRR-NH2 | 62 |
| 41 | Ac-TRVARTGRSRWRDVARNFMRAAOOOOOOOO-NH2 | 63 |
| 42 | Ac-TRVARTGRSRWRDVARNFMRAAKKKKKKKK-NH2 | 64 |
| 43 | Ac-TRVARTGRSRWRDVARNFMRRR-NH2 | 65 |
| 44 | Ac-TRVARTGRSRWRDVARNWMRRR-NN2 | 66 |
| 45 | Ac-ERVARTGRSRWRDVARNFMRRR-NH2 | 67 |
| 46 | Ac-AATRVARTGRSRWRDVARNFMRR-NH2 | 68 |
| 47 | Ac-RSVSNAATRVARTGRSRWRDVARNFMRR-NH2 | 69 |
| 48 | Ac-KKKKAATRVARTGRSRWRDVARNFMRAAKKKK-NH2 | 70 |
| 49 | H2N-RRRRAATRVARTGRSRWRDVARNFMRAARRRR-NH2 | 71 |
| 50 | betaAla-TRVARTGRSRWRDVARNFMRAA-NH2 | 72 |
| 51 | Ac-TRVARTGRSRWRDVARNFMRLL-NH2 | 73 |
| 52 | Ac-trvartgrsrwrdvarnfmraa-NH2 | 74 |
| 53 | Ac-trvartgrsrwrdvarnfmraakkkkkkkk-NH2 | 75 |
| 54 | Ac-trvartgrsrwrdvarnfmraaoooooooo-NH2 | 76 |
| 55 | H2N-kkkkkkkkaatrvartgrsrwrdvarnfmr-NH2 | 77 |
| 56 | Ac-trvartgrsrwrdwarnfmr-NH2 | 78 |
| 57 | Ac-trvartgrsrwrdvarnfmr-NH2 | 79 |
| 58 | Ac-trvartgrsrwrdvarnfmrkkkkkkkk-NH2 | 80 |
| 59 | Ac-trvartgrsrwrdvarnfmroooooooo-NH2 | 81 |
| 60 | Ac-trvartgrsrwrdvarnfar-NH2 | 82 |
| 61 | Ac-trvsrtgrsrwrdwsrnfar-NH2 | 83 |
| 62 | Ac-trvsrtgrsrwrdwsrnfmrc-NH2 | 84 |
| 63 | Lauryl-TRVSRTGRSRWRDWSRNFMR-NH2 | 85 |
| 64 | Lauryl-TRVSRTGRSRWRDWSRNFMRK-NH2 | 86 |
| 65 | Ac-TRVSRTGRSRWRDWSRNFMRK(Lauryl)-NH2 | 87 |
| 66 | Lauryl-TRVSRTGRSRWRDWSRNFMRK(Lauryl)-NH2 | 88 |
| 67 | Stearyl-TRVSRTGRSRWRDWSRNFMR-NH2 | 89 |
| 68 | Lauryl-TRVSRTGRSRWRDWSRNFMRLL-NH2 | 90 |
| 69 | Lauryl-TRVARTGRSRWRDVARNFMR-NH2 | 91 |
| 70 | Lauryl-TRVARTGRSRWRDWARNFMR-NH2 | 92 |
| 71 | Lauryl-TRVARTGRSRWRDVARNFMRK-NH2 | 93 |
| 72 | Ac-TRVARTGRSRWRDVARNFMRK(Lauryl)-NH2 | 94 |
| 73 | Lauryl-TRVARTGRSRWRDVARNFMRK(Lauryl)-NH2 | 95 |
| 74 | Lauryl-TRVARTGRSRWRDVARNFMRLL-NH2 | 96 |
| 75 | Stearyl-TRVARTGRSRWRDVARNFMR-NH2 | 97 |
| 76 | Lauryl-trvsrtgrsrwrdwsrnfmr-NH2 | 98 |
| 77 | Lauryl-trvartgrsrwrdwarnfmr-NH2 | 99 |
| 78 | Lauryl-trvartgrsrwrdvarnfmr-NH2 | 100 |
| 79 | Lauryl-trvsrtgrsrwrdwsrnfmrc-NH2 | 101 |
| 80 | Lauryl-trvsrtgrsrwrdwsrnfmraaK-NH2 | 102 |
| 81 | Stearyl-trvsrtgrsrwrdwsrnfmraaK-NH2 | 103 |
| 82 | Lauryl-trvartgrsrwrdvarnfmraak-NH2 | 104 |
| 83 | Stearyl-trvartgrsrwrdvarnfmraak-NH2 | 105 |
| 84 | Ac-trvsrtgrsrwrdwsrnfmraaK(Ac)-NH2 | 106 |
| 85 | Lauryl-trvsrtgrsrwrdwsrnfmraaK(Ac)-NH2 | 107 |
| 86 | Stearyl-trvsrtgrsrwrdwsrnfmraaK(Ac)-NH2 | 108 |
| 87 | Lauryl-RSRWRDWSRNFMR-NH2 | 109 |
| 88 | Lauryl-RSRWRDVARNFMR-NH2 | 110 |
| 89 | Lauryl-rsrwrdwsrnfmr-NH2 | 111 |
| 90 | Lauryl-rsrwrdvarnfmr-NH2 | 112 |
| 91 | Ac-RSRWRDWSRNFMR-NH2 | 113 |
| 92 | Ac-RSRWRDVARNFMR-NH2 | 114 |
| 93 | Ac-RSRWRDVSRNFMR-NH2 | 115 |
| 94 | Ac-RSRWRDWARNFMR-NH2 | 116 |
| 95 | Ac-rsrwrdwsrnfmr-NH2 | 117 |
| 96 | Ac-rsrwrdvarnfmr-NH2 | 118 |
| 97 | Lauryl-RSRWRDVARNFAR-NH2 | 119 |
| 98 | Lauryl-RSRWRDVARNFM-NH2 | 120 |
| 99 | Lauryl-RSRWRDVARNF-NH2 | 121 |
| 100 | Lauryl-RSRWRDVARN-NH2 | 122 |
| 101 | Lauryl-RSRWRDVAR-NH2 | 123 |
| 102 | Lauryl-RSRWRDVA-NH2 | 124 |
| 103 | Lauryl-RSRWRDV-NH2 | 125 |
| 104 | Lauryl-SRWRDVA-NH2 | 126 |
| 105 | Lauryl-RWRDVAR-NH2 | 127 |
| 106 | Lauryl-WRDVARN-NH2 | 128 |
| 107 | Lauryl-RDVARNF-NH2 | 129 |
| 108 | Lauryl-DVARNFM-NH2 | 130 |
| 109 | Lauryl-VARNFMR-NH2 | 131 |
| 110 | Lauryl-DVARNFMR-NH2 | 132 |
| 111 | Lauryl-RDVARNFMR-NH2 | 133 |
| 112 | Lauryl-WRDVARNFMR-NH2 | 134 |
| 113 | Lauryl-RWRDVARNFMR-NH2 | 135 |
| 114 | Lauryl-SRWRDVARNFMR-NH2 | 136 |
| 115 | Lauryl-rsrwrdvarfm-NH2 | 137 |
| 116 | Lauryl-rsrwrdvarnf-NH2 | 138 |
| 117 | Lauryl-rsrwrdvarn-NH2 | 139 |
| 118 | Lauryl-rsrwrdvar-NH2 | 140 |
| 119 | Lauryl-rsrwrdva-NH2 | 141 |
| 120 | Lauryl-rsrwrdv-NH2 | 142 |
| 121 | Lauryl-srwrdva-NH2 | 143 |
| 122 | Lauryl-rwrdvar-NH2 | 144 |
| 123 | Lauryl-wrdvarn-NH2 | 145 |
| 124 | Lauryl-rdvarnf-NH2 | 146 |
| 125 | Lauryl-dvarfm-NH2 | 147 |
| 126 | Lauryl-varnfmr-NH2 | 148 |
| 127 | Lauryl-dvarnfmr-NH2 | 149 |
| 128 | Lauryl-rdvarnfmr-NH2 | 150 |
| 129 | Lauryl-wrdvarnfmr-NH2 | 151 |
| 130 | Lauryl-rwrdvarnfmr-NH2 | 152 |
| 131 | Lauryl-srwrdvarnfmr-NH2 | 153 |
| 132 | Lauryl-rsrlrdllrnlar-NH2 | 154 |
| 133 | Lauryl-rsrwrdllrnlmr-NH2 | 155 |
| 134 | Lauryl-RSRWRDVLRNFMR-NH2 | 156 |
| 135 | Lauryl-RSRWRDVARNFLR-NH2 | 157 |
| 136 | Lauryl-RSRWRDVLRNFLR-NH2 | 158 |
| 137 | Lauryl-RSRWRDVARNFMRLL-NH2 | 159 |
| 138 | Lauryl-RSRWRDVLRNFLRLL-NH2 | 160 |
| 139 | Lauryl-RSRWRDVARNWMR-NH2 | 161 |
| 140 | Lauryl-RSRWRDWARNFMR-NH2 | 162 |
| 141 | Lauryl-RSRWRDVARNFMRW-NH2 | 163 |
| 142 | Lauryl-RSRWRDVARNFMRWW-NH2 | 164 |
| 143 | Lauryl-RSRWRDWARNWMR-NH2 | 165 |
| 144 | Lauryl-RSRWRDWARNWMRW-NH2 | 166 |
| 145 | Lauryl-RSRWRDWARNWMRWW-NH2 | 167 |
| 146 | Lauryl-RSRWRDWARNWMRLL-NH2 | 168 |
| 147 | Lauryl-RSRWRDWWRNWMR-NH2 | 169 |
| 148 | Lauryl-RSRWRDWWRNWMRLL-NH2 | 170 |
| 149 | Lauryl-RSRZRDVARNFMR-NH2 Z = 1-naphthylalanine | 171 |
| 150 | Lauryl-RSRWRDZARNFMR-NH2 Z = 1-naphthylalanine | 172 |
| 151 | Lauryl-RSRWRDVZRNFMR-NH2 Z = 1-naphthylalanine | 173 |
| 152 | Lauryl-RSRWRDVARNZMR-NH2 Z = 1-naphthylalanirie 174 | |
| 153 | Lauryl-RSRZRDZARNFMR-NH2 Z = 1-naphthylalanine | 175 |
| 154 | Lauryl-RSRZRDVARNZMR-NH2 Z = 1-naphthylalanine | 176 |
| 155 | Lauryl-RSRZRDZARNZMR-NH2 Z = 1-naphthylalanine | 177 |
| 156 | Lauryl-RSRWRDZARNZMR-NH2 Z = 1-naphthylalanine | 178 |
| 157 | Lauryl-RSRZRDZARNZMRZZ-NH2 Z = 1-naphthylalanine | 179 |
| 158 | Lauryl-RsRwRdvaRnfmR-NH2 | 180 |
| 159 | Lauryl-xsxwxdvaxnfmx-NH2 x = D-homoarginine | 181 |
| 160 | Lauryl-XSXZXDZAXNZMXZ-NH2 | 182 |
| | X = homoarginine, Z = 1-naphthylalanine | |
| 161 | Lauryl-RSRWRDVARNFL-NH2 | 183 |
| 162 | Lauryl-SRWRDVARNFLR-NH2 | 184 |
| 163 | Lauryl-RWRDVARNFLR-NH2 | 185 |
| 168 | H2N-RSRWRDVARNFLR-Ethylamidohexanyl | 186 |
| 169 | H2N-RSRWRDVARNFLR-Ethylamidooctanyl | 187 |
| 170 | H2N-RSRWRDVARNFLR-Ethylamidodecanyl | 188 |
| 171 | H2N-RSRWRDVARNFLR-Ethylamidolauryl | 189 |
| 172 | H2N-RSRWRDVARNFLR-Ethylamidopalmitoyl | 190 |
| 173 | H2N-RSRWRDVARNFMR-Ethylamidohexanyl | 191 |
| 174 | H2N-RSRWRDVARNFMR-Ethylamidooctanyl | 192 |
| 175 | H2N-RSRWRDVARNFMR-Ethylamidodecanyl | 193 |
| 176 | H2N-RSRWRDVARNFMR-Ethylamidolauryl | 194 |
| 177 | H2N-RSRWRDVARNFMR-Ethylamidopalmitoyl | 195 |
| 178 | H2N-RSRWRDVARNFLRK(Hexanyl)-NH2 | 196 |
| 179 | H2N-RSRWRDVARNFLRK(Octanyl)-NH2 | 197 |
| 180 | H2N-RSRWRDVARNFLRK(Decanyl)-NH2 | 198 |
| 181 | H2N-RSRWRDVARNFLRK(Lauryl)-NH2 | 199 |
| 182 | H2N-RSRWRDVARNFLRK(Palmitoyl)-NH2 | 200 |
| 183 | H2N-RSRWRDVARNFMRK(Hexanyl)-NH2 | 201 |
| 184 | H2N-RSRWRDVARNFMRK(Octanyl)-NH2 | 202 |
| 185 | H2N-RSRWRDVARNFMRK(Decanyl)-NH2 | 203 |
| 186 | H2N-RSRWRDVARNFMRK(Lauryl)-NH2 | 204 |
| 187 | H2N-RSRWRDVARNFMRK(Palmitoyl)-NH2 | 205 |
| 188 | Hexyl-rsrwrdvarnflr-NH2 | 206 |
| 189 | Octyl-rsrwrdvarnflr-NH2 | 207 |
| 190 | Decanyl-rsrwrdvarnflr-NH2 | 208 |
| 191 | Palmitoyl-rsrwrdvarnflr-NH2 | 209 |
| 192 | H2N-rsrwrdvarnflrk(Hexanyl)-NH2 | 210 |
| 193 | H2N-rsrwrdvarnflrk(Octanyl)-NH2 | 211 |
| 194 | H2N-rsrwrdvarnflrk(Decanyl)-NH2 | 212 |
| 195 | H2N-rsrwrdvarnflrk(Lauryl)-NH2 | 213 |
| 196 | H2N-rsrwrdvarnflrk(Palmitoyl)-NH2 | 214 |
| 197 | H2N-rsrwrdvarnfmrk(Hexanyl)-NH2 | 215 |
| 198 | H2N-rsrwrdvarnfmrk(Octanyl)-NH2 | 216 |
| 199 | H2N-rsrwrdvarnfmrk(Decanyl)-NH2 | 217 |
| 200 | H2N-rsrwrdvarnfmrk(Lauryl)-NH2 | 218 |
| 201 | H2N-rsrwrdvarnfmrk(Palmitoyl)-NH2 | 219 |
| 202 | Hexyl-RSRWRDVARNFMR-NH2 | 220 |
| 203 | Octyl-RSRWRDVARNFMR-NH2 | 221 |
| 204 | Decanyl-RSRWRDVARNFMR-NH2 | 222 |
| 205 | Palmitoyl-RSRWRDVARNFMR-NH2 | 223 |
| 206 | Hexyl-rsrwrdvarnfmr-NH2 | 224 |
| 207 | Octyl-rsrwrdvarnfmr-NH2 | 225 |
| 208 | Decanyl-rsrwrdvarnfmr-NH2 | 226 |
| 209 | Palmitoyl-rsrwrdvarnfmr-NH2 | 227 |
| 210 | Lauryl-rsrwrdvarnflr-NH2 | 228 |
| 211 | Lauryl-rsrwrdvarnfl-NH2 | 229 |
| 212 | Lauryl-srwrdvarnflr-NH2 | 230 |
| 213 | Lauryl-RSRWRDVARNFLR-NH2, all amino acids are beta3 | 231 |
| 214 | Lauryl-RWSRVDRANRFLR-NH2, all amino acids are beta3 | 232 |
| 215 | H2N-RWSRVDRANRFLR-Ethylamidolauryl | 233 |
| | all amino acids are beta3 | |
| 216 | H2N-RWSRIDRINRFLR-Ethylamidolauryl | 234 |
| | all amino acids are beta3 | |
| 217 | Lauryl-rarwravarafar-NH2 | 235 |
| 218 | Lauryl-rlrwrlvlrlflr-NH2 | 236 |
| 219 | Lauryl-rkrwrdvarnflr-NH2 | 237 |
| 220 | Lauryl-rkrwrevarnflr-NH2 | 238 |
| 221 | Lauryl-rsrwrevarnflr-NH2 | 239 |
| 222 | Lauryl-rrrwrrvarrflr-NH2 | 240 |
| 223 | Lauryl-rkrwrkvarkflr-NH2 | 241 |
| 224 | Lauryl-rarwrdvarnflr-NH2 | 242 |
| 225 | Lauryl-rsrwravarnflr-NH2 | 243 |
| 226 | Lauryl-rsrwrdvaraflr-NH2 | 244 |
| 227 | Lauryl-rlrwrevarlflr-NH2 | 245 |
| 228 | Lauryl-rsawrdvarnfmr-NH2 | 246 |
| 229 | Lauryl-rsrwadvarnfmr-NH2 | 247 |
| 230 | Lauryl-rsrwrdvaanfmr-NH2 | 248 |
| 231 | Lauryl-rsrwrdvarnfma-NH2 | 249 |
| 232 | Lauryl-rsrsrdvarnfmr-NH2 | 250 |
| 233 | Lauryl-rsrwrdsarnfmr-NH2 | 251 |
| 234 | Lauryl-rsrwrdvarnsmr-NH2 | 252 |
| 235 | Lauryl-rsrwrdvarnfsr-NH2 | 253 |
| 236 | Lauryl-asrwrdvarnfmr-NH2 | 254 |
| 237 | Lauryl-rarwrdvarnfmr-NH2 | 255 |
| 238 | Lauryl-rsawrdvarnfmr-NH2 | 256 |
| 239 | Lauryl-rsrardvarnfmr-NH2 | 257 |
| 240 | Lauryl-rsrwadvarnfmr-NH2 | 258 |
| 241 | Lauryl-RWRDLAR-NH2 | 259 |
| 242 | Lauryl-RWRALAR-NH2 | 260 |
| 243 | Lauryl-RWRAVAR-NH2 | 261 |
| 244 | Lauryl-rwrdlar-NH2 | 262 |
| 245 | Lauryl-rwralar-NH2 | 263 |
| 246 | Lauryl-rwravar-NH2 | 264 |
| 247 | Lauryl-rsrwrdvarnfmrc-NH2 | 265 |
| 248 | Ahx-rsrwrdvarnfmr-NH2 | 266 |
| 249 | Lauryl-rsrwrdvardfmr-NH2 | 267 |
| 250 | Ac-TRVARTGRSRWRDWARNFM-NH2 | 268 |
| 251 | Hexyl-rsrwrdvarnflr-NH2 | 269 |
| 252 | Octyl-rsrwrdvarnflr-NH2 | 270 |
| 253 | Decanyl-rsrwrdvarnflr-NH2 | 271 |
| 254 | Palmityl-rsrwrdvarnflr-NH2 | 272 |

Peptide amino acid sequences are presented in one letter codes, where lowercase letters designate a D-isomer and uppercase letters designate an L-isomer. In the table, “Ac” is acetyl, “NH2” is amido, “H2N” is amino, “O” is ornithine. “X,” “Y,” and “Z” designations in the amino acid sequences of Table 3 are defined within the table and sometimes designate different amino acids than when the designations are utilized in sequence motifs described previously. Where “Z” is a designation for diamino propionic acid (DAP), DAP can be crosslinked to an aspartic acid moiety within a peptide. When a crosslink is used, numerical notations are used to indicate which individual amino acid is crosslinked (e.g., D4 and D15 refer to the Asp residue in the 4th and 15t^{h} positions as counted from the amino terminus, or left side, of the sequence). Peptide compositions not listed in Table 3 can be derived by combining features from peptide compositions listed explicitly in Table 3. For example, a portion of one peptide composition, such as a N-terminal modification moiety, C-terminal modification moiety, lipophilic moiety and/or peptide amino acid sequence, can be exchanged for a counterpart portion from another peptide composition in Table 3 (e.g., a N-terminal modification of one peptide composition can be exchanged for a N-terminal modification of another peptide composition, and an amino acid sequence of one peptide composition can be exchanged with the amino acid sequence of another peptide composition). Also, one or more D-amino acids may be exchanged for L-amino acids, or one or more L-amino acids may be exchanged for D-amino acids. A lipophilic moiety may be oriented at another portion of the peptide other than explicitly shown in Table 3, and may be substituted with a different lipophilic moiety, examples of which are described hereafter.

Lipophilic Molecules

As described above, a peptide moiety in a composition sometimes is linked to one or more lipophilic molecules (e.g., hydrophobic molecule) that increase the hydrophobicity of the peptide in the peptide composition. The hydrophobicity of a lipophilic molecule sometimes is expressed in terms of a log p value. Log p values are derived from octanol/water partitioning studies, in which molecules with higher hydrophobicity partition into octanol with higher frequency and are characterized as having a higher log p value. Log p values are published for a number of lipophilic molecules and log p values can be calculated using known partitioning processes (e.g., Chemical Reviews, Vol. 71, Issue 6, page 599, where entry 4493 shows lauric acid having a log p value of 4.2).

In some embodiments, the lipophilic molecule has a log p value of +1 to +6, and sometimes has a log p value of +3 to +4.5. Any lipophilic moiety can be linked to a peptide composition described above and tested for antimicrobial activity using known methods and those described hereafter. The lipophilic moiety sometimes is a C1-C20 alkyl, C2-C20 alkenyl, C2-C20 alkynyl, C3-C6 cycloalkyl, C1-C4 haloalkyl, C4-C12 cyclalkylalkyl, aryl, substituted aryl, or aryl(C1-C4) alkyl, for example. In some embodiments, two C6 alkyl moieties are linked to a peptide moiety in a composition. The lipophilic molecule sometimes is an acyl-containing moiety, which in some embodiments is a fatty acid moiety. Any acyl-containing moiety or fatty acid moiety can be utilized that results in a peptide composition having antimicrobial activity. Examples of fatty acid acyl-containing moieties are propyl (C3), butyl (C4), pentyl (C5), hexyl (C6), heptyl (C7), octyl (C8), nonyl (C9), decyl (C10), undecyl (C11), lauryl (C12), myristyl (C 14), palmityl (C16), stearyl (C18), arachidyl (C20), behenyl (C22) and lignoceryl moieties (C24), and each moiety can contain 0, 1, 2, 3, 4, 5, 6, 7 or 8 unsaturations (i.e., double bonds). In specific embodiments, the lipophilic moiety is a lauryl moiety.

The lipophilic moiety often is linked to the peptide by a covalent linkage and sometimes by a non-covalent linkage. The lipophilic moiety sometimes is linked to the peptide by an amide linkage, and the linkage sometimes is to the peptide N-terminus, the peptide C-terminus or a side chain of an amino acid within the peptide composition (e.g., a lysine or ornithine side chain). The lipophilic molecule sometimes is linked to the peptide via a non-amide linkage, which includes but is not limited to a carbon-carbon linkage. The lipophilic moiety is joined to the peptide composition using known methods, examples of which are described hereafter. For example, multiple methods of joining the lipophilic moiety to the N-terminus of a peptide are known, such as reacting an alkyl halide form of the lipophilic molecule with the N-terminus of the peptide, or joining the lipophilic molecule via known peptide synthetic procedures in which the peptide is on a solid support. For example, the lipophilic moiety sometimes is linked to the peptide as if it were another amino acid added to the peptide, such as in a method described in the Examples section hereafter. Linkages, linkers, and functional groups useful for covalently conjugating a peptide to a lipophilic moiety are described, for example, in U.S. Pat. Nos. 6,387,628 and 6,589,485. Methods for non-covalently linking a lipophilic moiety to a peptide also are known, including but not limited to, derivitizing the peptide or the lipophilic molecule with one or more biotin molecules and derivitizing the peptide or lipophilic molecule not linked to biotin to avidin or streptavidin, and then joining the derivitized peptide and lipophilic moiety.

When linked to a peptide composition, a lipophilic moiety sometimes localizes (e.g., selectively delivers or accumulates) the composition on and/or in skin substructures and components (e.g., one or more of those described above) as compared to a composition comprising a peptide moiety not linked to a lipophilic moiety. Determining whether the peptide composition is localized or selectively delivered to a skin substructure or component sometimes is determined using a process described hereafter.

Pharmaceutical Compositions

Provided herein are pharmaceutical compositions comprising peptide compositions described above and a pharmaceutically acceptable carrier. Any pharmaceutically acceptable carrier can be formulated with the peptide compositions so long as the peptide composition retains all or some antimicrobial activity. Determining whether the peptide composition retains antimicrobial activity when formulated with a carrier is performed using antimicrobial assays known in the art and disclosed herein. Examples of pharmaceutically acceptable carriers include but are not limited to a carrier, a diluent, an excipient, an auxiliary, a binder, a lubricant, a colorant, a disintegrant, a buffer, an isotonic agent, a preservative, an anesthetic, and the like which are used in a medical field. Pharmaceutical compositions comprising the peptide compositions may be manufactured by any known method, including but not limited to conventional mixing, dissolving, granulating, dragee-making, levigating, emulsifying, encapsulating, entrapping or lyophilizing processes.

The pharmaceutically acceptable carrier often is selected in part by the administration route for the composition. For example, routes of administration include but are not limited to topical administration, eye dropping, instillation, percutaneous administration, injection (e.g., subcutaneous, intracutaneous, intravenous, intraperitoneal), oral administration, inhalation, and the like. Also, the dosage form such as injectable preparations (e.g., solutions, suspensions, emulsions, solids to be dissolved), tablets, capsules, granules, powders, liquids, liposome inclusions, ointments, gels, washes, pads, patches, cosmetics, external powders, sprays, inhaling powders, eye drops, eye ointments, suppositories, pessaries, and the like often are selected in part on the administration method.

For topical administration, a peptide composition may be formulated as an ointment, cream, gel, lotion, paste, and the like. Examples of components in such compositions are discussed in U.S. Pat. Nos. 6,245,342; 6,139,850; 6,042,848; 6,333,042; 6,358,929; 6,455,076; 6,509,014; 6,558,695; 6,582,724; 6,602,856; and 6,630,572, for example.

Ointments often are semisolid preparations based on petrolatum or other petroleum derivatives. The specific ointment base to be used is one that will provide for optimum drug delivery, and often will provide other desired characteristics as well, e.g., emolliency or the like. As with other carriers or vehicles, an ointment base often is inert, stable, nonirritating and nonsensitizing. As explained in Remington: The Science and Practice of Pharmacy, 19th Ed. (Easton, Pa.: Mack Publishing Co., 1995), at pages 1399-1404, ointment bases sometimes are grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Water-soluble ointment bases sometimes are prepared from polyethylene glycols of varying molecular weight (e.g., Remington: The Science and Practice of Pharmacy for further information).

Creams often are viscous liquids or semisolid emulsions, and often are oil-in-water or water-in-oil. Cream bases are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

Gels often are semisolid, suspension-type systems. Single-phase gels contain organic macromolecules distributed substantially uniformly throughout the carrier liquid, which is typically aqueous, and sometimes also contain an alcohol and, optionally, an oil. Gelling agents sometimes are crosslinked acrylic acid polymers such as the “carbomer” family of polymers, e.g., carboxypolyalkylenes that may be obtained commercially under the Carbopol® trademark. Gelling agents sometimes are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, carboxymethylcellulose, carboxymethylcellulose sodium, and methylcellulose; gums such as tragacanth and xanthan gum; sodium alginate; and gelatin. Dispersing agents such as alcohol or glycerin can be added, or the gelling agent can be dispersed by trituration, mechanical mixing or stirring, or combinations thereof, to prepare a uniform gel. Further examples of gelling agents include but are not limited to a poloxamer, polyvinyl alcohol, methyl hydroxybenzoate, ethyl hydroxybenzoate, propyl hydroxybenzoate, butyl hydroxybenzoate, and the like.

Lotions are preparations often applied to the skin surface without friction, and are typically liquid or semiliquid preparations in which solid particles, including the active agent, are present in a water or alcohol base. Lotions often are suspensions of solids, and sometimes comprise a liquid oily emulsion of the oil-in-water type. Lotions often are utilized for treating large body areas, and facial areas, because of the ease of applying a fluid composition. Any insoluble matter in a lotion often is finely divided. Lotions typically contain suspending agents to produce dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes sometimes are divided between fatty pastes or those made from single-phase aqueous gels. The base in a fatty paste often is petrolatum or hydrophilic petrolatum or the like. Pastes made from single-phase aqueous gels sometimes incorporate carboxymethylcellulose or the like as a base.

Formulations sometimes are prepared with liposomes, micelles, and microspheres. Liposomes are microscopic vesicles having a lipid wall comprising a lipid bilayer, and can be used as drug delivery systems. Liposome formulations sometimes are utilized for poorly soluble or insoluble peptide compositions. Liposomal preparations include cationic (positively charged), anionic (negatively charged) and neutral preparations. Cationic liposomes are readily available. For example, N[1-2,3-dioleyloxy)propyl]-N,N,N-triethylammonium (DOTMA) liposomes are available under the tradename Lipofectin®. (GIBCO BRL, Grand Island, N.Y.). Anionic and neutral liposomes also are readily available, e.g., from Avanti Polar Lipids (Birmingham, Ala.), or can be readily prepared using available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with DOTMA in appropriate ratios. Methods for making liposomes using these materials are known.

Micelle formulations often comprise surfactant molecules arranged so that their polar headgroups form an outer spherical shell, while the hydrophobic, hydrocarbon chains are oriented towards the center of the sphere, forming a core. Micelles often form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl-8-dodecyl ether, polyoxyl-12-dodecyl ether, nonoxynol 10 and nonoxynol 30.

Like liposomes and micelles, microspheres often encapsulate a peptide composition in some formulations. They are generally although not necessarily formed from lipids, often charged lipids such as phospholipids. Preparation of lipidic microspheres is known and described in pertinent texts and literature.

Various additives sometimes are included in topical formulations. For example, a solvent, (e.g., an alcohol) sometimes is used to solubilize peptide compositions in the formulation. Other optional additives include opacifiers, antioxidants, fragrance, colorant, gelling agents, thickening agents, stabilizers, surfactants and the like. Other agents sometimes are added, such as antimicrobial agents, to prevent spoilage upon storage, i.e., to inhibit growth of microbes such as yeasts and molds in the formulation. Suitable antimicrobial agents sometimes are selected from the group consisting of the methyl and propyl esters of p-hydroxybenzoic acid (i.e., methyl and propyl paraben), sodium benzoate, sorbic acid, imidurea, and combinations thereof.

One or more permeation enhancers sometimes are included in the formulation. Permeation enhancers sometimes minimize the possibility of skin damage, irritation, and systemic toxicity. Examples of permeation enhancers include, but are not limited to, ethers such as diethylene glycol monoethyl ether (available commercially as Transcutol) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin (U.S. Pat. No. 4,783,450; alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as polyethylene glycol, and polyethylene glycol monolaurate (PEGML; see, e.g., U.S. Pat. No.4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly citric acid and succinic acid. Azone® and sulfoxides such as DMSO and C.sub.10 MSO may also be used. Penetration enhancers are discussed in Percutaneous Penetration Enhancers, eds. Smith et al. (CRC Press, 1995).

Formulations sometimes contain irritation-mitigating additives to minimize or eliminate the possibility of skin irritation or skin damage resulting from a formulated peptide composition or other components of the formulation. Examples of irritation-mitigating additives include, but are not limited to, alpha.-tocopherol; monoamine oxidase inhibitors, including phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. An irritant-mitigating additive, if present, sometimes is incorporated into the present formulations at a concentration effective to mitigate irritation or skin damage, sometimes representing less than about 20 percent by weight, and often less than about 5 percent by weight, of the formulation.

The concentration of the peptide composition in the formulation can vary, and will depend on a variety of factors, including the disease or condition to be treated, the nature and activity of the peptide composition, the desired effect, possible adverse reactions, the ability and speed that the peptide composition reaches its intended target, and other factors within the particular knowledge of the patient and physician.

The peptide composition structure in the formulation or before formulation sometimes is characterized as not having substantial alpha helical content in certain embodiments (i.e., substantially free or free of alpha helix structure). Methods for determining the helical content of a peptide composition are known, such as by utilizing circular dichroism spectrophotometric analysis for example. A peptide composition sometimes contains 50% or less alpha helical content, 40% or less alpha helical content, 30% or less alpha helical content, 20% or less alpha helical content, 10% or less alpha helical content, 5% or less alpha helical content, or 1% or less alpha helical content. Alpha helix content can be measured for a peptide composition in water, a solvent, or a combination thereof, such as water and acetonitrile (e.g., 20% acetonitrile), optionally including other agents, such as a buffer agent for example.

In some embodiments, the peptide composition structure in the formulation or before formulation is not substantially helix-turn-helix. The peptide structure in some embodiments may consist of a random, non-helical structure; a helix; a helix and a non-helical region at one end of the helix; or a helix with a non-helix region at each end of the helix.

In certain embodiments, the pharmaceutical composition is an ointment applied by topical administration. In an embodiment, the pharmaceutical composition comprises one or more of the following components: sorbitan monostearate, polyoxyethylene sorbitan monostearate, isopropyl palmitate, vaseline, liquid paraffin, cetanol, glycerol, magnesium stearate and water. In specific embodiments, the composition includes the following components: 10 mg of a peptide composition, 7 mg sorbitan monostearate, 7 mg polyoxyethylene sorbitan monostearate, 37 mg isopropyl palmitate, 37 mg vaseline, 37 mg liquid paraffin, 50 mg cetanol, 70 mg glycerol, 2 mg magnesium stearate and water in an amount to prepare 1 g of ointment.

In some embodiments, the pharmaceutical composition comprises one or more gel agents. Such pharmaceutical compositions often include a vehicle, including but not limited to purified water USP, alcohol USP (95%), or the like, and sometimes contain a preservative, such as methylparaben, propylparaben and the like. Such pharmaceutical compositions sometimes include a buffer, including but not limited to a phosphate buffer system (if compatible) for pH 7.0 to 7.4, sodium dihydrogen phosphate, disodium hydrogen phosphate, phosphoric acid and the like, or a potassium form of the forgoing. Examples of gel formulations comprising the peptide composition include but are not limited to the following: (1) xanthan gum, sodium chloride, potassium phosphate, sodium hydroxide, sodium methyl p-hydroxybenzoate, sodium propyl p-hydroxybenzoate and purified water; (2) methyl hydroxybenzoate 0.8 mg/g, propyl hydroxybenzoate 0.2 mg/g, disodium edetate, carbomer, propylene glycol, sodium hydroxide to adjust pH and purified water q.s. to 100% w/w; (3) hydroxyethylcellulose, propylene glycol, sodium citrate, methyl hydroxybenzoate, disodium edetate, propyl hydroxybenzoate, citric acid and purified water; (4) diethanolamine, oxyethylenated hydrogenated fatty acid esters, isopropanol, monoglycerides and diglycerides of fatty acids, carbomer, methyl p-hydroxybenzoate, propyl p-hydroxybenzoate, butylhydroxytoluene, aroma and purified water; and (5) ethyl p-hydroxybenzoate 0.1%,butyl p-hydroxybenzoate 0.10%, lauromacrogol 0.50%, cetanol 18.00%, white petrolatum 40.00%, distilled water 40.85%, 1-monomyristoyl-rac-glycerol 0.30%.

In certain embodiments, a peptide composition gel formulation comprises one or more of the following components: a solvent (e.g., ethanol); a humectant (e.g., propylene glycol, which can serve as a moisturizer (it is hygroscopic)); a penetration enhancer (e.g., isopropylmyristate); a stabilizer (e.g., EDTA); an antioxidant (e.g., a vitamin such as vitamin A and/or E, sialicylic acid) and an acid (e.g., HCl or H₂SO₄) or base (e.g., NaOH) to adjust the pH of the formulation. In certain embodiments, the peptide composition is formulated with (% wt/wt) 0.001% to 5% peptide composition (e.g., 3.0% or less, 2.5% or less, 2% or less, 1% or less, 0.5% or less, 0.1% or less, or 0.05% or less), 0% to 50% alcohol (e.g., about 20% ethanol), 0.01% to 5% hydroxypropylcellulose, 0% to 10% propylene glycol, 0% to 2% isopropyl myristate, 0% to about 1% EDTA disodium (e.g., about 0.01% to about 0.5%, or 0.5% or less, 0.25% or less, or 0.1% or less) and an amount of base, such as 1N NaOH to adjust the pH between 3 and 7. In an embodiment, the peptide composition is formulated with (% wt/wt) 0.1% to 2.5% peptide composition, 30% ethanol, 2% hydroxypropylcellulose, 5% propylene glycol, 0.5% isopropyl myristate, 0.01%, 0.1%, 0.25% or 0.5% EDTA disodium and an amount of a base, such as 1N NaOH, to adjust the pH to 4.5.

In other embodiments, the pharmaceutical composition is a tablet, which at times is ingested and sometimes is crushed and applied by topical administration. In addition to one or more of the peptide compositions or conjugates described herein as an active ingredient, the tablet sometimes comprises one or more of the following components: lactose, potato starch, crystalline cellulose and light silicic anhydride. In specific embodiments, the tablet comprises 100 mg of a peptide composition, 670 mg lactose, 150 mg potato starch, 60 mg crystalline cellulose and 50 mg light silicic anhydride. The components sometimes are mixed and after kneading with addition of a solution of 30 mg of hydroxypropylcellulose in methanol (10% by weight of hydroxypropylcellulose), the mixture sometimes is granulated. The mixture sometimes is extruded through a 0.8 mm-diameter screen to form granules. After drying, 15 mg of magnesium stearate sometimes is added and the mixture is tabulated in amounts of 200 mg each. In certain embodiments, the pharmaceutical composition is a capsule, which at times is ingested, and sometimes is crushed and the contents applied by topical administration. The capsule in certain embodiments includes a peptide composition described above in combination with other ingredients, such as lactose for example. In a specific embodiment, the capsule contents are 100 mg of a peptide composition and 80 mg lactose in a hard shell or soft gel capsule.

In some embodiments, topical administration of the pharmaceutical composition sometimes is coupled with delivering an electric current to the area the pharmaceutical composition is applied. For example, a pharmaceutical composition is applied topically to the skin of a subject and an electric current is applied to the area, and sometimes around the area, of the skin on which the pharmaceutical composition is deposited. The electric current sometimes is applied before, sometimes during, and often after administration of the pharmaceutical composition. Appropriate apparatus for generating current are known and specific aspects of currents useful for facilitating delivery of the pharmaceutical composition (e.g., current amplitude, voltage amplitude, electric field amplitude, electric field orientation, the frequency of reorienting the electric field if it is reoriented, whether pulses are utilized, and the number and duration of pulses) are known (e.g., U.S. Pat. Nos. 6,654,636; 6,009,345 and 5,704,908). The electric current may enhance delivery of the peptide composition to the skin according to any mechanism, such as electrophoresis and/or iontophoresis, for example.

For oral administration, the peptide compositions can be readily formulated by combining the active peptides or peptide analogues with pharmaceutically acceptable carriers well known in the art. Such carriers enable the peptide compositions of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, such as lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, and the like. Additionally, flavoring agents, preservatives, coloring agents and the like may be added.

Systemic formulations include those designed for administration by injection, e.g. subcutaneous, intravenous, intramuscular, intrathecal or intraperitoneal injection, as well as those designed for transdermal, transmucosal, oral or pulmonary administration. For injection, the peptide compositions of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Alternatively, the peptide compositions may be in powder form for constitution with a suitable vehicle, e.g., sterile pyrogen-free water, before use.

For transmucosal administration, penetrants appropriate to the barrier to be permeated are used in the formulation. Such penetrants are generally known in the art. For buccal administration, the peptide compositions may take the form of tablets, lozenges, and the like. formulated in conventional manner.

For administration by inhalation, the peptide compositions for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the peptide composition and a suitable powder base such as lactose or starch.

The peptide compositions also may be formulated in rectal or vaginal compositions such as suppositories or retention enemas, e.g, containing conventional suppository bases such as cocoa butter or other glycerides.

In addition to the formulations described previously, the peptide compositions also may be formulated as a depot preparation. Such long acting formulations may be administered by implantation (for example subcutaneously or intramuscularly) or by intramuscular injection. Thus, for example, the peptide compositions may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the peptide compositions may be delivered using a sustained-release system, such as semipermeable matrices of solid polymers containing the therapeutic agent. Various sustained-release materials have been established and are well known by those skilled in the art. Sustained-release capsules may, depending on their chemical nature, release the peptide compositions for a few weeks up to over 100 days. Depending on the chemical nature and the biological stability of the therapeutic reagent, additional strategies for protein stabilization may be employed.

As the peptide compositions may contain charged side chains or termini, they may be included in any of the above-described formulations as the free acids or bases or as pharmaceutically acceptable salts. Pharmaceutically acceptable salts are those salts which substantially retain the antimicrobial activity of the free bases and which are prepared by reaction with inorganic or organic (e.g., salicylate, tartrate) acids. Pharmaceutical salts tend to be more soluble in aqueous and other protic solvents than are the corresponding free base forms.

Pharmaceutical compositions sometimes are a combination of a peptide composition described above with one or more other agents that enhances the effectiveness of the composition. For example, a pharmaceutical composition for treating acne may include a peptide composition described herein in combination with a topical comedolytic (e.g., benzoyl peroxide, salicylic acid, tretinoin, azelaic acid, tretinoin, adapalene); topical antibiotic (erythromycin, clindamycin, genatmycin, metronidazole, sodium sulfacetamide); oral antibiotic (e.g., tetracycline, doxycycline, minocycline, erythromycin, amoxicillin, caphalexin); hormonal therapeutic or diuretic (e.g., low androgenic activity oral contraceptive, norgestimate, desogestrel, spironolactone); alpha hydroxy acid; an antioxidant (e.g., vitamin A, C and/or E; retinoid (e.g., retinol)); an anti-inflammatory agent; an analgesic; or combinations of the foregoing.

The peptide compositions generally are used in an amount effective to achieve the intended purpose (e.g., reduce microbial populations, reduce inflammation and treat acne). When used to treat or prevent acne, the composition is administered or applied in a therapeutically effective amount. A therapeutically effective amount is an amount effective to ameliorate or prevent the acne symptoms of the subject being treated. The therapeutically effective amount sometimes treats, prevents, reduces and/or ameliorates a symptom or cause of acne, such as pustule eruption; comedone development; papule development; excess sebum production, excess production of keratinocytes, outlet obstruction of sebaceous follicle; increased proliferation of P. acnes; inflammation; folliculitis; cellulitis; keloid development; acne conglobata symptoms such as development of nodules, cysts, abscesses and severe scarring; and hyperpigmentation (see e.g., Woodward, Topics in Advanced Practice Nursing eJournal 2 (2002) at http address www.medscape.com/viewarticle/430534). A therapeutically effective amount sometimes is determined in part by assays described herein. For example, a dose can be formulated and tested in skin assays to determine an IC.sub.50 value for reducing bacterial populations in the skin. Such information can be used to more accurately determine useful doses.

Dosage amount and interval may be adjusted individually to provide peptide composition levels sufficient to maintain a therapeutic effect. Patient dosages for topical administration range from about 0.01 mg/day to about 100 mg/day. Patient dosages for administration by injection or oral administration range from about 0.1 to 5 mg/kg/day, preferably from about 0.5 to 1 mg/kg/day. Therapeutically effective levels may be achieved by administering multiple doses each day.

The amount of peptide composition administered often is independent on the subject being treated, the severity of the affliction, the manner of administration and the judgment of the prescribing physician. The dosage may vary within this range depending upon the dosage form employed and the route of administration utilized. The exact formulation, route of administration and dosage can be chosen by the individual physician in view of the patient's condition. (see e.g., Fingl et al., 1996, In: The Pharmacological Basis of Therapeutics, 9.sup.th ed., Chapter 2, p. 29, Elliot M. Ross). The therapy may be repeated intermittently while symptoms are detectable or when they are not detectable. The therapy may be performed by administering the peptide composition in combination with one or more other agents that enhances the effectiveness of the composition for treating acne, examples of which are described above.

A therapeutically effective dose of the peptide compositions described herein will provide a therapeutic benefit without causing substantial toxicity. Toxicity of the peptide compositions described herein can be determined by standard pharmaceutical procedures in cell cultures or experimental animals, and assays described hereafter can be utilized to determine doses that yield a toxic effect. Sometimes, a therapeutically effective amount is guided by identifying a LD.sub.50 value, which is the dose lethal to 50% of the population, or a LD.sub.100, which is the dose lethal to 100% of the population. The dose ratio between toxic and therapeutic effect is the therapeutic index. Peptide compositions which exhibit high therapeutic indices are preferred. The data obtained from cell culture assays and animal studies can be used to formulate a dosage range that is not toxic for use in humans.

Methods of Using Antimicrobial Compositions

There are many uses for the peptide compositions and pharmaceutical compositions described herein (collectively referred to hereafter as “compositions”). For example, featured herein is a method for reducing a microbe population in a system, which comprises administering a composition to the system in an amount that reduces the microbe population. The composition often comprises an antimicrobial peptide described herein, which sometimes linked to a lipophilic moiety. In certain embodiments the microbe is a bacterium, a yeast, a fungus or a virus. In other embodiments, the population is a component of a microbe, such as lipopolysaccharide (LPS) or an endotoxin. Bacteria sometimes are Gram-negative (e.g., Escherichia coli, Klebsiella and Salmonella), sometimes are Gram-positive (e.g., Staphylococcus aureus), at times are drug resistant Gram-positive bacteria (e.g., methicillin-resistant Staphylococcus aureus (MRSA), methicillin-sensitive Staphylococcus aureus (MSSA) and vancomycin-resistant enterococci) and sometimes are drug resistant Gram-negative bacteria (e.g., multiple drug resistant Helicobacter, Shigella and Salmonella). In certain embodiments, the bacterium is selected from the group consisting of Salmonella, Staphylococcus, Propionibacterium, Escherichia, Pseudomonas, Pityrosporum, Candida and Trichophyton, and in specific embodiments, is selected from the group consisting of Salmonella dublin, Staphylococcus aureus, Propionibacterium acnes, Escherichia coli, Pseudomonas aeruginosa, Staphylococcus epidermidis, Pityrosporum ovale, Candida albicans and Trichophyton rubrum. In certain embodiments, the yeast is from Pityrosporum (e.g., Pityrosporum ovate) or Candida (e.g., Candida albicans), and in other embodiments the fungus is filamentous, such as a filamentous fungus from Trichophyton (e.g., Trichophyton rubrum).

Any known system that maintains a microbial population or allows growth of a microbial population can be utilized. The system sometimes is a solid matrix that contains a growth medium suitable for monitoring populations of the target microbe (e.g., a flask, petri dish or inoculation tube containing a solid, semi-solid or liquid growth medium suitable for maintaining one or more target microbes). In certain embodiments, the system is the skin of an animal, such as a human, another mammal (e.g., dogs, cats, and ungulates (e.g., cattle, sheep, and swine)), an avian (e.g., chickens and turkeys), a reptile, or a fish (e.g., salmon and trout), for example. In specific embodiments, the system is human skin, and often the composition is delivered by topical administration, or sometimes by injection. The skin sometimes is tested after it is removed from a subject (e.g., often on and/or in skin from a non-human animal), as performed in certain assays described hereafter, and often the composition is administered to skin not removed from the subject (i.e., the skin is integral with the subject).

Reduction of a microbial population is determined using a method for detecting microbes in a system, and such methods are known and are described hereafter. These methods sometimes include counting microbial colonies on a cell culture plate, and may include detecting DNA or RNA sequences specific to a microorganism. A microbial population is reduced when a system contacted with a composition includes fewer microbes than a system not contacted with the composition. The number of microbes in the system sometimes is determined or estimated, and often relative microbial populations are determined with reference to a system not contacted with a composition described herein. The population of one type of microbe sometimes is assessed, and sometimes populations of two or more microorganisms are assessed (e.g., the system medium may support the growth of more than one microbe and one or a few may predominate). In embodiments where a microbial population on and/or in skin is assessed, the population may be determined in a skin sample removed from a subject, in a substructure of skin (e.g., in a pore, a blocked pore and/or a sebaceous gland), or in a skin component (e.g., sebum and/or keratin). The population often is reduced if the number of microbes in a system contacted with the composition is 90% or less, 80% or less, 70% or less, 60% or less, 50% or less, 40% or less, 30% or less, 20% or less, 10% or less, 1% or less, 0.1% or less, 0.01% or less, or 0.001% or less than in a system not contacted with the composition.

In an embodiment, a reduction in microbial population is assessed in a method which comprises isolating sebum from the skin of a subject, contacting the sebum in a system with a composition described herein, and determining whether the composition reduces the microbial population. Determining whether the composition reduces the microbial population in the system often is determined by comparison with a microbial population in a system not contacted with the composition. The microbial population in some embodiments is Propionibacterium acnes with or without other microbes capable of growing under anaerobic conditions. In certain embodiments, the sebum is isolated using a cosmetic product (e.g., a removable strip), and sometimes the cosmetic product is contacted with the skin and the sebum is removed from the product for further processing. In some embodiments, the sebum is incubated in an anaerobic culture system, and often microbial populations (e.g., colonies) are determined.

Also featured is a method for reducing inflammation in a tissue of a subject, which comprises administering a composition to the tissue in an amount that reduces the inflammation, where the composition comprises an antimicrobial peptide described herein, optionally linked to a lipophilic moiety. The tissue often is skin, sometimes human skin, and the composition often is delivered by topical administration to the skin. In some embodiments, inflammation is assessed visually (e.g., the degree of acne lesions is scored visually using known techniques). A reduction in inflammation sometimes is assessed by determining whether a cell type and/or biological molecule associated with inflammation is modified (e.g., reduced macrophage, interleukin-1, tumor necrosis factor-alpha and/or gamma-interferon levels are associated with a reduction in inflammation), and sometimes is determined relative to a system not contacted with the composition. Also featured is a method of inhibiting a bacterial lipase in the skin of a subject, which comprises administering a composition to the skin in an amount that inhibits the bacterial lipase, where the composition comprises an antimicrobial peptide described herein, which sometimes is linked to a lipophilic moiety. Lipase inhibition sometimes is assessed by detecting a reduction in the conversion of lipids to free fatty acids, and sometimes is assessed by detecting a reduction in inflammation in the skin since free fatty acids produced by bacterial lipases often cause skin inflammation.

Featured also is a method for treating a medical condition or a microbe-causing complication of a medical condition, often a skin condition, which comprises administering a composition to skin of a subject in an amount that treats the condition, where the composition comprises an antimicrobial peptide described herein, which sometimes is linked to a lipophilic moiety. In some embodiments, the medical condition is rosacea, atopic dermatitis (e.g., eczema), a Candida infection (e.g., vaginal, diaper, intertrigo, balanitis, oral thrush), Tinea versicolor, Dermatophytosis (e.g., Tinea pedis (athlete's foot), Tinea unguium (nails), Onychomycosis (toe nail fungus), Tinea cruris (groin), Tinea capitus (scalp), Tinea corporis (nonhair-bearing skin: ringworm; scalp: kerion), Tinea barbae (beard-area)), seborrheic dermatitis, antibiotic-resistant skin infections, impetigo, ecthyma, erythrasma, burn wounds (e.g., reduction of infections, improved healing), diabetic foot/leg ulcers (e.g., reduction of infections, improved healing), prevention of central catheter-related blood stream infections, oral mucositis, warts (e.g., common, flat, plantar, genital), and molluscum contagiosum. In certain embodiments, the condition is acne, often acne vulgaris and sometimes acne conglobate. The composition often is delivered by topical administration to the skin, and the subject often is human. Also provided is a method for treating a medical condition or a microbe-causing complication of a medical condition, which comprises administering a composition comprising an antimicrobial peptide described herein, which sometimes is linked to a lipophilic moiety. Examples of administration include but are not limited to pulmonary, parenteral and intravenous administration. The medical condition can be any condition caused by a microbe (e.g., pneumonia, sepsis) or a microbe-causing complication of any medical condition not caused by a microbe or treatment thereof (e.g., a microbial complication of cystic fibrosis).

Also featured is a method for selectively delivering an antimicrobial composition to a skin substructure or component (e.g., sebum, keratin, one or more sebaceous glands, one or more open pores and/or blocked pores, one or more open comedones and/or closed comedones, one or more pilosebaceous units), which comprises administering a composition to the skin in an amount that selectively delivers the composition to the skin component or substructure, where the composition comprises an antimicrobial peptide described herein, which sometimes is linked to a lipophilic moiety. The composition often is delivered by topical administration to the skin, the skin sometimes is not integrated with a subject (i.e., the skin is removed from the subject), the skin often is integrated in a subject (i.e., the skin is not removed from the subject), and the skin often is human skin. Methods for determining whether components of the composition are delivered to skin substructures and components are known and a method is described hereafter.

In an embodiment, selective delivery of components of a composition is determined in a method which comprises administering a peptide composition to skin of a subject, where the peptide in the composition is linked to a detectable label, and determining whether the peptide composition is localized in a particular skin substructure or component. Selective delivery of a peptide composition to a skin substructure or component often is attained when a higher concentration of the peptide composition is present in the target skin structure or component as compared to other skin structures or components. In specific embodiments, the peptide composition concentration in sebum, a sebaceous gland and/or blocked pore is 2 times or more, 3 times or more, 4 times or more, 5 times or more, 6 times or more, 7 times or more, 8 times or more 9 times or more, 10 times or more, 20 times or more, 50 times or more, or 100 times or more greater than the concentration in another skin substructure or component. The peptide composition is linked to any useful detectable label, including but not limited to a radioactive isotope (e.g., ¹²⁵I, ¹³¹I, ³⁵S, ³²P, ³³P, ¹⁴C or ³H); a light scattering label (e.g., U.S. Pat. No. 6,214,560); a fluorophore (e.g., Anantha et al., Biochemistry 37: 2709-2714 (1998); Qu & Chaires, Methods Enzymol 321:353-69 (2000)), a chemiluminescent molecule; an enzymic or protein label (e.g., GFP or peroxidase); or other chromogenic label or dye (e.g., Texas Red). Any method for detecting the label can be utilized (e.g., a microscopy method using skin samples or detection method in solution using seburn samples) to determine if a peptide composition is localized to a particular skin substructure or component.

In certain embodiments, a peptide composition described herein is applied to a surface of a device to prevent microbial proliferation on that surface of the device. The device often is a medical device, which includes any material or device that is used on, in, or through a patient's body in the course of medical treatment (e.g., for a disease or injury). Medical devices include but are not limited to such items as medical implants, wound care devices, drug delivery devices, and body cavity and personal protection devices. The medical implants include but are not limited to urinary catheters, intravascular catheters, dialysis shunts, wound drain tubes, skin sutures, vascular grafts, implantable meshes, intraocular devices, heart valves, prosthetic devices (e.g., hip prosthetics) and the like. Wound care devices include but are not limited to general wound dressings, biologic graft materials, tape closures and dressings, and surgical incise drapes. Drug delivery devices include but are not limited to needles, drug delivery skin patches, drug delivery mucosal patches and medical sponges. Body cavity and personal protection devices include but are not limited to tampons, sponges, surgical and examination gloves, and toothbrushes. Birth control devices include but are not limited to intrauterine devices (IUDs), diaphragms, and condoms

Skin Mounting Apparatus

In embodiments where the system is a portion of skin removed from a subject, provided is an apparatus useful for mounting the skin after excision. As explained above, a skin sample from a subject often rounds after excision, making it difficult to manipulate the sample. The apparatus overcomes this technical difficulty by flattening the skin sample. Biological reagents are contacted with the skin mounted in the apparatus through channels in the top plate described in further detail below. Biological agents include microbial preparations and peptide compositions described herein. The apparatus can be utilized in processes involving skin, such as in methods for reducing a microbial population in or on skin described hereafter.

In general, the apparatus often comprises a pair of plates each having a flat surface, where the flat surface of one plate is mated to a flat surface on the other plate. The dimensions of the two surfaces having the largest surface area on each plate often are identical, and the mating surface of each plate typically has identical dimensions. Each plate has a thickness that yields a stiff surface (e.g., yielding plates that flex insubstantially when one end is fixed and a force is applied to the other end), and the thickness of the top plate sometimes is less than the thickness of the bottom plate. Similarly, the plates are constructed from any material that yields stiff plates. The plates often are constructed from a material that is readily cleaned and sterilized, where skin samples and biological reagents are readily removed from the plates with water and mild cleaning agents and the plates are not damaged when exposed to sterilizing ultraviolet irradiation conditions or sterilization conditions with 100% ethanol. The plates sometimes are constructed from a material that allows the plates to be sterilized under high temperatures and/or high pressures without becoming deformed (e.g., resistant to physical perturbations under autoclaving conditions). The plates often are constructed from a plastic, and in some embodiments, the plates are constructed from acrylic.

The top plate includes several circular openings, extending from the top surface of the plate to the bottom surface of the plate, forming channels between the circular openings at the top surface and circular openings at the bottom surface. The diameter of the circular openings on the top surface of the plate often is between 0.1 mm to 10 mm, and sometimes is between 3mm to 5 mm, between 6 mm to 8 mm, between 1.5 mm to 2 mm, 4.5 mm or 7 mm. The channel often is cylindrical, where the wall of the cylindrical opening is vertical with respect to the top surface of the top plate. In other embodiments, the channel is a tapered cylinder, sometimes with the circular opening at the top surface of the plate having a larger diameter than the diameter of the circular opening on the bottom surface of the plate. The top plate includes any number of circular openings and channels in any orientation. In certain embodiments, the channels are arranged in a square grid, sometimes in a five-by-five array (i.e., 25 channels) and sometimes in an eight-by-eight array (i.e., 64 channels). In certain embodiments, the top plate has the dimensions 10 cm by 10 cm by 1 cm thick.

The bottom plate also includes several circular openings that form wells suited to receive a medium for preserving the skin sample. An example of a suitable medium is Dulbecco's Modified Eagle Medium (D-MEM) with or without L-glutamine. The circular openings on the top surface of the bottom plate are of the same diameter of the circular openings on the bottom surface of the top plate and the circular openings on the bottom surface of the top plate and the top surface of the bottom plate are arranged to align when the top and bottom plates are mated. Thus, the bottom plate has the same number of wells as channels in the top plate and they are in the same spatial orientation (e.g., where the top plate has a five-by-five array of channels, the bottom plate has a five-by-five array of wells arranged in the same orientation). Each well often terminates within the bottom plate and does not extend through the plate to the bottom surface of the plate, unlike the channels in the top plate. Wells often terminate at a point located at about half the plate thickness. Wells can be any shape suitable for containing a liquid medium. In some embodiments, the well is partially cylindrical, where the cylindrical portion extends from the circular opening in the top surface of the bottom plate and having the same diameter as the circular opening, and is partially conical, where the conical portion extends from the bottom of the cylindrical portion and terminates within the plate. The end (i.e., tip) of the conical portion of the well in such embodiments often terminates around the middle of the bottom plate thickness (e.g., where the bottom plate is 2 cm thick, the tip of the conical portion often is located around 1 cm below the top surface of the bottom plate). In certain embodiments, the conical portion of the well is substituted with a rounded conical portion (i.e., the tip is not a point but a rounded surface), and in other embodiments, the conical portion is substituted with a cylindrical portion having a flat bottom (i.e., the bottom of the cylindrical well is parallel to the top surface and bottom surface of the bottom plate) or a cylindrical portion having a rounded bottom (e.g., shaped like a standard test tube). In certain embodiments, the bottom plate has the dimensions 10 cm by 10 cm by 2 cm thick.

The top and bottom plates are joined to one another using any suitable fastener that applies a pressure between the plates sufficient to avoid any leakage of liquid medium between the wells when a skin sample is mounted between the plates in the apparatus. Examples of fasteners include but are not limited to clamps and threaded screws. The fasteners are constructed from any suitable material capable of maintaining a pressure that avoids substantial leakage of liquid medium when a skin sample is mounted (e.g., a plastic or metal). Where the fastener is a screw, the head of the screw often is configured to allow fastening by a commercially available device, such as a screw driver of any convenient configuration (e.g., flat head, Phillips head or hexagonal head). In certain embodiments, the fastener is a threaded screw constructed from stainless steel. In embodiments in which threaded screws are utilized as fasteners, the top and bottom plates include channels of an appropriate diameter and shape to hold the screws at a pressure noted above. In such embodiments, the top plate often includes channels located at the periphery, extending from the top surface of the plate to the bottom surface of the plate, each channel running through the entire width of the top plate. In such embodiments, each channel is adapted to receive a screw, where the channel often is counter-threaded to receive the threads of each screw. The bottom plate includes the same number of channels as the top plate, where the channels extend through a partial thickness of the bottom plate and terminate within the plate thickness (e.g., often terminating at a location about half the thickness of the plate). The channels in the bottom plate are oriented to align with the channels in the top plate such that a screw driven through the top plate enters a channel in the bottom plate. The channels in the bottom plate also are adapted to receive each of the screws. In certain embodiments, a gasket constructed from a flexible or semi-flexible material (e.g., plastic or rubber) is oriented between the top surface of the top plate and the bottom surface of the fastener. In certain embodiments, the gasket is ring-shaped and a screw fastener is passed through it such that the gasket lies between the bottom of the screw head and the top surface of the top plate when the screw is fastened.

The fasteners often are applied with a pressure sufficient to avoid substantial leakage from well/channel pairs. Leakage sometimes is determined by observing fluid patterns on a skin sample or test membrane sample mounted in the device (e.g., by loading a dye in each channel and/or well pair) and observing any spreading beyond the circumference of the circular openings of the channel/well pairs. Insubstantial leakage often is leakage 1 mm to 2 mm or less beyond the circumference of each circular opening.

The skin sample excised from a subject is placed on one of the mating surfaces of the top or bottom plate, often the mating surface of the bottom plate, and the plates are assembled (i.e., mated) and joined using a fastener. The skin sample is from any subject, including a mouse or a human subject (e.g., a human cadaver). A single skin sample often is large enough to cover each well in the bottom plate of the apparatus, and in other embodiments, multiple skin samples are assembled in one apparatus.

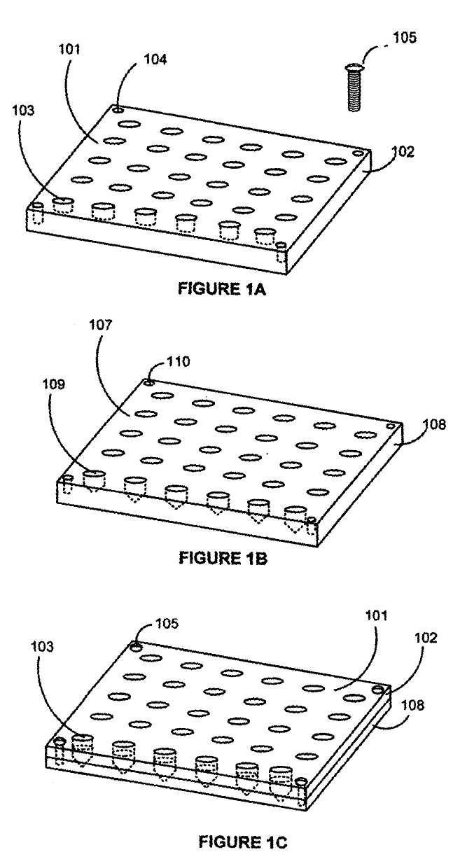

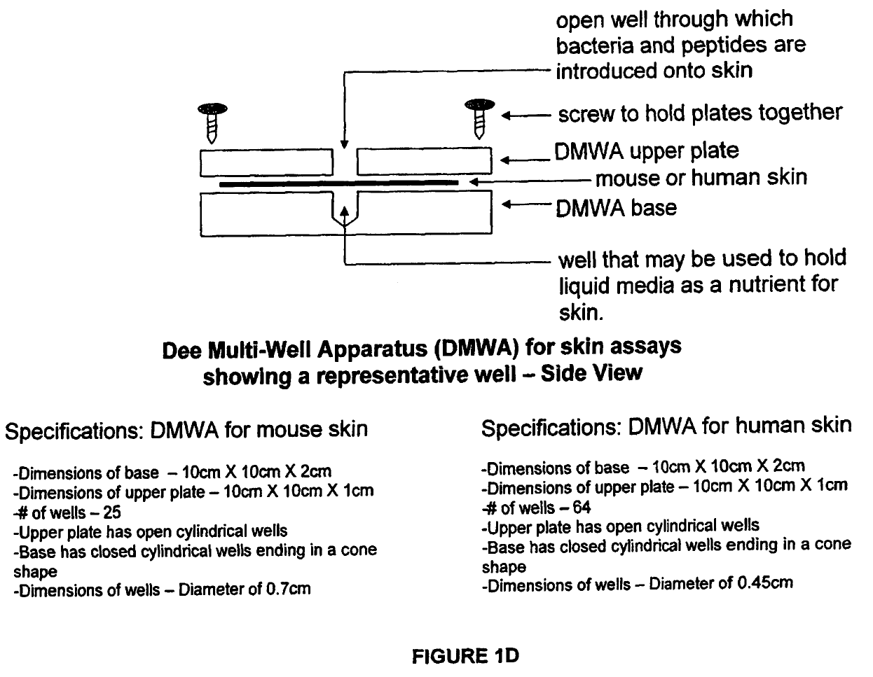

In specific apparatus embodiments illustrated in FIGS. 1A and 1B, the top plate shown in FIG. 1A has a top surface 101 and a plate thickness 102. The bottom surface has the same dimensions and surface area as the top surface in the top plate. Dimensions of the surface 101 often are 10 cm by 10 cm and the thickness 102 often is 1 cm. Cylindrical channels having a circular opening 103 emanate downward through the top surface 101 of the top plate and exit the bottom surface of the plate with a bottom circular opening having the same diameter as the top circular opening. Thirty cylindrical channels are arranged in a rectangular five-by-six array, and as described above, the apparatus can include other channel/well configurations, such as five-by-five and eight-by-eight arrays. The top plate often includes four cylindrical channels 104 that pass through the entire thickness 102 of the plate, one in each comer of the plate, and are adapted to receive screw fasteners 105 (e.g., the channels 104 are counter-threaded to engage threads on the screw fasteners 105).

A bottom plate embodiment shown in FIG. 1B has a top surface 107 and a plate thickness 108. The top surface of the bottom plate often is of the same dimensions and surface area as the bottom surface of the plate, and often is of the same dimensions and surface area as the top and bottom surfaces of the top plate. The top surface 107 of the bottom plate often is 10 cm by 10 cm and the thickness 108 often is 2 cm. The bottom plate includes wells terminating within the plate thickness 108, often at a point about half of the plate thickness 108 (e.g., terminating about 1 cm from the top surface of a bottom plate having a total thickness of 2 cm). As shown in the FIG. 1B, the shape of the well often is defined by a circular opening 109, is partially cylindrical as it extends downward from the circular opening in the top surface of the plate, and is partially conical as it extends from the cylindrical portion and terminates within the plate. The circular openings 109 of the wells are located in the bottom plate in the same configuration as the channels 103 in the top plate. The bottom plate includes cylindrical channels adapted to receive screw fasteners, having circular openings aligned with the circular openings in the bottom surface of the bottom plate, and terminating within the thickness 108 of the bottom plate. The channels adapted to receive screw fasteners often terminate at a point located about half the distance of the plate thickness 108.

FIG. 1C shows an embodiment of an assembled apparatus containing a skin sample (the skin sample is not shown). The top plate in FIG. 1A and the bottom plate in FIG. 1B are mated and fastened using screws 105, and the channels defined by the circular openings 103 in the top plate are aligned with the wells defined by the circular openings 109 in the top surface of the bottom plate.

FIG. 1D shows a representational side view of an apparatus embodiment as it is being assembled, and certain apparatus characteristics for mouse skin and human skin applications. FIG. 1D shows a representation of one channel and one well described above and provides a representation of skin mounted in the device.

Thus, featured is an apparatus which comprises a top plate and a bottom plate, one or more fasteners, and a skin sample, where the skin sample is mounted between the top plate and the bottom plate, the top plate comprises one or more channels each defined by a circular opening in the top and bottom surfaces of the top plate, the bottom plate comprises one or more wells each having a circular opening on the top surface aligned with a circular opening of a channel on the bottom surface of the top plate, and the top plate and bottom plate are joined by the one or more fasteners.

### EXAMPLES

The examples set forth below illustrate and not limit the invention.

### Example 1

### Synthesizing Peptide Compositions

The following methods were utilized to synthesize peptide compositions described herein. All Fmoc-protected alpha-amino acids and Rink amide resin were purchased from EMD Biosciences/Novabiochem (San Diego, Calif.). N-Methylpyrrolidinone (NMP), dimethylformamide (DMF), diisopropylethylamine (DIEA), piperidine, trifluoroacetic acid (TFA), CH3CN, and 2-(1H-benzotriazole-1-yl)-1,1,3,3-tetramethyluronium hexafluorophosphate (HBTU) were purchased from American Bioanalytical (Natick, Mass.). Acetic anhydride, CH₂Cl₂, triisopropylsilane (TIPS), ethanedithiol, thioanisole, phenol, alpha-cyanohydroxycinnamic acid (CHCA), and lauric acid were purchased from Sigma-Aldrich (Milwaukee, Wis.). Et₂O was purchased from Pharmco Products (Brookfield, Conn.). Purification was performed on a Varian PrepStar HPLC (dual Model 218 pump modules and Model 320 UV-Vis detector) with a Thomson reversed-phase preparative column (C18, Advantage 300 Angstrom, 5 micron, 20 mm×250 mm, 15 mL/min flow rate). Final analysis was performed on a Waters analytical HPLC system (Waters 2695 separations module and Waters 2996 photodiode array detector) with a Vydac analytical HPLC column (C18, 300 Angstrom, 5 micron, 4.6 mm×150 mm, 1 mL/min flow rate) using UV detection at 214 nm. Separations were performed using linear gradients of solvent B (95% CH₃CN in water, with 0.05% TFA) in solvent A (5% CH3CN in water, with 0.05% TFA). Mass spectra were acquired on a Bruker Autoflex MALDI-TOF mass spectrometer (Bruker Daltonics, Inc.) in CHCA matrix.

Peptides were synthesized via methodology standard in the field. Specifically, synthesis was achieved on a Rainin/PTI Symphony automated peptide synthesizer using standard synthesis cycles (double coupling, Ac₂O capping) with HBTh activation and standard Fmoc-based amino acid derivatives. Peptide cleavage was in a cocktail consisting of 90% TFA, and 2% each of water, triisopropylsilane, ethanedithiol, thioanisole, and phenol for 2.5 h. Crude peptides were purified to homogeneity and lyophilized to dryness to yield the peptide as a trifluoroacetate salt. In some cases, the peptide is passed through an ion exchange column to convert the peptide to an alternative salt form, such as the HCl salt. Peptide identity was confirmed by MALDI-TOF mass spectrometry.

Attachment of a lipid tail such as lauric acid to the peptide sometimes was achieved by direct coupling of the lipid (i.e., hexanoate, laurate, stearate) to the N-terminus of the peptide as the final step of the solid phase synthesis. Sometimes the lipid was attached via a lysine side chain that was suitably protected and deprotected (orthogonally to the standard amino acid side chain protecting groups and the peptide resin) during the solid phase synthesis. In the latter method of attachment, the lysine side chain could be anywhere in the peptide sequence. Lipids could also be attached via a cysteine side chain by disulfide and thioether bonds, or to an acidic side chain by employing an amine-derivative of a lipid molecule.

Peptides having D-amino acids were synthesized using standard methods, and a procedure for synthesizing a representative peptide (Peptide Number 76, SEQ ID 98), Lauryl-[D]Thr-[D]Arg-[D]Val-[D]Ser-[D]Arg-[D]Thr-Gly-[D]Arg-[D]Ser-[D]Arg-[D]Trp-[D]Arg-[D]Asp-[D]Trp-[D]Ser-[D]Arg-[D]Asn-[D]Phe-[D]Met-[D]Arg-NH₂ is described. Peptide assembly was achieved via a standard method at a scale of 50 micromoles. In addition to standard washes with NMP, repeated cycles included the following steps: Fmoc deprotection was achieved with four successive six min washes of 20% piperidine in DMF, chain elongation was performed via two 25 min couplings in NMP with five equivalents (250 micromoles) of HBTU-activated Fmoc-amino acid relative to initial resin loading (Rink amide resin, 0.43 mmole/g, 116 mg, 50 micromoles), and unreacted peptide chains were then capped with a five min wash of 10% Ac2O/10% DIEA in NMP. Side chain protecting groups on the Fmoc-amino acids were utilized as follows: tert-Butyl for Ser/Thr/Asp, Pbf for Arg, Boc for Trp, and trityl for Asn. Upon completion of chain assembly, the N-terminal Fmoc protecting group was removed and lauric acid was coupled via the HBTU-activated derivative in an identical manner as the Fmoc-amino acids. After final washes with NMP, CH₂Cl₂, and MeOH, the resin was dried under a stream of nitrogen. A cocktail containing TFA (2.25 milliliters), ethanedithiol (0.05 milliliters), TIPS (0.05 milliliters), thioanisole (0.05 milliliters), water (0.05 milliliters), and phenol (50 mg) was added to the dry resin and the reaction was agitated intermittently by nitrogen flow, as performed in the standard protocol on the Rainin Symphony instrument. The TFA cocktail containing crude, deprotected peptide was poured into a 50 mL polypropylene tube containing 35 mL Et₂O at −80 degrees C. to precipitate the crude peptide. The tube was centrifuged at 3000 rpm for 5 minutes and the supernatant was decanted away from the crude peptide pellet. Et2O (−80 degrees C.) was added to the tube to a volume of 35 mL and the tube was vigorously shaken to distribute the crude peptide. The tube was again centrifuged and the supernatant decanted. A stream of nitrogen was applied to the wet peptide pellet to remove excess Et₂O until cracks appeared on the surface. The crude peptide was dissolved in HPLC buffer A and purified via a linear 0-100% gradient of B in A over 30 min. Fractions identified by MALDI-TOF MS to contain the product were pooled, lyophilized, and repurified to yield the final product as a single peak by analytical HPLC. The material was lyophilized to dryness and used in experiments.

Another representative peptide(Peptide Number 65, SEQ ID 87), Ac-[D]Thr-[D]Arg-[D]Val-[D]Ser-[D]Arg-[D]Thr-Gly-[D]Arg-[D]Ser-[D]Arg-[D]Trp-[D]Arg-[D]Asp-[D]Trp-[D]Ser-[D]Arg-[D]Asn-[D]Phe-[D]Met-[D]Arg-[D]-Lys(epsilon-Lauryl)-NH2, was synthesized using the following procedure. Peptide assembly was achieved via a standard method at a scale of 50 micromoles. In addition to standard washes with NMP, repeated cycles included the following steps: Fmoc deprotection was achieved with four successive six min washes of 20% piperidine in DMF, chain elongation was performed via two 25 min couplings in NMP with five equivalents (250 micromoles) of HBTU-activated Fmoc-amino acid relative to initial resin loading (Rink amide resin, 0.43 mmole/g, 116 mg, 50 micromoles), and unreacted peptide chains were then capped with a five min wash of 10% Ac₂O/10% DIEA in NMP. Sidechain protecting groups on the Fmoc-amino acids were utilized as follows: tert-Butyl for Ser/Thr/Asp, Pbf for Arg, Boc for Trp, and trityl for Asn, with the single exception of the C-terminal Lys. The C-terminal Lys residue was added to the resin as the Fmoc-D-Lysine(Mtt)-OH derivative. The orthogonal protecting group [Mtt, or (4-methyl)-triphenylmethyl] was removed from the fully protected peptide-resin using 1% TFA, 5% TIPS in CH₂Cl₂. The TFA solution was washed over the resin for approximately 15-20 minutes via gravity filtration. The eluate was initially bright yellow, and progressively turned lighter as the removal of Mtt proceeded. Once the eluate was colorless, the resin was washed with CH₂Cl₂ and NMP, resulting in a protected peptide-resin with a free Lys side chain at the C-terminus. Lauric acid was coupled directly to the free amine side chain of this Lys via the HBTU-activated derivative in an identical manner as the Fmoc-amino acids. After final washes with NMP, CH₂Cl₂, and MeOH, the resin was dried under a stream of nitrogen. A cocktail containing TFA (2.25 milliliters), ethanedithiol (0.05 milliliters), TIPS (0.05 milliliters), thioanisole (0.05 milliliters), water (0.05 milliliters), and phenol (50 milligrams) was added to the dry resin and the reaction was stirred 3 h. Filtered TFA cocktail containing crude, deprotected peptide was poured into a 50 milliliters polypropylene tube containing 35 milliliters Et₂O at −80 degrees C. to precipitate the crude peptide. The tube was centrifuged at 3000 rpm for 5 minutes and the supernatant was decanted away from the crude peptide pellet. Et₂O (−80 degrees C.) was added to the tube to a volume of 40 milliliters and the tube was vigorously shaken to distribute the crude peptide. The tube was again centrifuged and the supernatant decanted. A stream of nitrogen was applied to the wet peptide pellet to remove excess Et₂O until cracks appeared on the surface. The crude peptide was dissolved in HPLC buffer A and purified via a linear 0-100% gradient of B in A over 30 min. Fractions identified by MALDI-TOF MS to contain the product were pooled and lyophilized to yield the final product as a single peak by analytical HPLC. The material was lyophilized to dryness and used in experiments.

The following procedure for attaching a lipophilic moiety or other chemical moiety to the C-terminal backbone of an antimicrobial peptide sometimes is utilized. A Mmt protecting group is removed from the Universal NovaTag™ resin (EMD, Novabiochem, San Diego, Calif.) using 1% TFA and 5% triisopropylsilane in dichloromethane (10 successive washes of 2 minutes each). The molecule intended for C-terminal backbone attachment (a carboxylic acid, alkyl halide or equivalent, or other molecule suitable for attachment to a primary amine) is coupled to the free amine resin by normal peptide synthesis methods (for coupling acids to amines) or by a suitable method compatible with the molecule of interest. Peptide elongation begins with initial removal of the Fmoc group and coupling of the first (C-terminal) amino acid; standard solid phase peptide synthesis techniques allow elongation of the desired peptide chain. When peptide elongation is complete and any relevant N-terminal modification is carried out, the peptide is removed from the resin using 90% TFA with 2% each of triisopropylsilane, ethanedithiol, thioanisole, water, and phenol. The resulting peptide is a fusion between the peptide (N-terminal) and the derivative of interest at the C-terminus, separated by an intervening ethyl moiety.

### Example 2

### Microbial Inhibition Assays for Determining Activity of Peptide Compositions

The following assays were conducted to determine antimicrobial activities for specific peptide compositions. These assays are routinely performed to assess antimicrobial activity of other peptide compositions.

Materials

S. dublin (Lane), S. aureus (Rosenbach), P. acnes, ATCC 6919, E. coli K12 TOP10, and E. coli K12 55099 (protease neg.) bacterial strains were tested in the microbial inhibition assays described hereafter. The S. dublin (Lane), and S. aureus (Rosenbach) strains are available from ATCC (Staphylococcus aureus subsp. aureus Rosenbach (Number: 13150) and Salmonella choleraesuis serotype dublin (Number: 39184)). The strain of P. acnes was purchased from ATCC (strain #6919) (Manassas, Va.). The E. coli K12 strains TOP 10 and 55099 were purchased from Invitrogen (Carlsbad, Calif.) and ATCC respectively. HeLa cells were purchased from ATCC (CCL2). Tryptic soy broth (TSB), Brucella broth, reinforced Clostridial broth and agar were purchased from Becton & Dickinson (distributed by VWR, West Chester, Pa.). All plastic consumables were purchased from VWR (West Chester, Pa.). Chemicals were purchased from Sigma Aldrich (St. Louis, Mo.). DMEM, antibiotics and cell culture supplements were purchased from Invitrogen (Carlsbad, Calif.), fetal calf serum (FCS) was purchased from Hyclone (Logan, Utah).

S. aureus and S. dublin were grown on TSB agar plates for 16 hours at 37° C. For propagation in liquid culture individual colonies were inoculated in 3 microliters of TSB broth for 16 hours at 37° C. under constant shaking. P. acnes was grown on Brucella broth blood agar plates (supplemented with 5% defibrinated sheep blood, 5 microgram/microliter hemin and 0.5 microgram/microliter vitamin K) under anaerobic conditions (GasPak system, Becton & Dickinson) for 96 hours at 37° C. For propagation in liquid culture individual colonies were inoculated in 3 microliters of reinforced Clostridial broth under anaerobic conditions for 72 hours at 37° C. HeLa cells were grown in DMEM substituted with 2 mM L-glutamine, 0.1 mM non-essential amino acids, 1 mM sodium pyruvate and 10% FCS. Cells were maintained in a humidified incubator at 37° C. and 5% CO₂.

Broth Microdilution Assay for Determination of MIC of Antimicrobial Peptide Compositions

The minimum inhibitory concentration (MIC) of peptide compositions for S. aureus, S. dublin and E. coli was determined in a broth microdilution assay. Two-fold serial dilutions of each peptide (volume 50 microliters) were prepared in 96-well plates with incubation medium (20% TSB, 50 mM Na₂CO₃, 1 mM MgCl₂). Each dilution series contained control wells (bacteria without peptide). A total of 50 microliters adjusted inoculum (105 bacteria) was added to each well. The microwell plates were then incubated in a humidified environment for 16 hours at 37° C. The MIC for each peptide for each microorganism was determined by three methods. The growth of bacteria was directly determined by measuring the absorption at 600 nm using a Versamax microplate reader (Molecular Devices). To confirm these results 10 microliters of a 1:100 dilution of each well was spotted onto a TSB agar plate. After incubation for 16 hours at 37° C. bacterial growth was evaluated and documented (Alpha Innotech gel documentation system).

The MIC is expressed as a 3 log or greater reduction in bacterial growth over a growth period of 16 hours in the presence of peptide compared to the negative control.

TABLE 4A
Minimum inhibitory concentration (MIC) of Granulysin
peptides for different bacteria species [MIC in micromolar units]

| Peptide in | | | E. coli | E. coli |
|---|---|---|---|---|
| Table 3 | S. aureus | S. dublin | Top10 | 55099 |
| 30 | 8 | 4 | 4 | 2 |
| 32 | 2 | 2 | 2 | 1 |
| 64 | 2 | 8 | 2 | 1 |

Broth Microdilution Assay for Determination of P. acnes MBC of Peptide Compositions

The minimum bactericidal concentration (MBC) of peptide compositions for P. acnes was determined in a microdilution assay. Two-fold serial dilutions for each peptide (volume 25 microliters) were prepared in a round bottom microwell plate with incubation medium (1% TSB, 10 mM NaH₂PO₄). Each dilution series included control wells containing bacteria without peptide. P. acnes was cultivated as described above. Three microliters of the culture was washed twice with incubation medium. The bacterial pellet was resuspended in incubation medium and adjusted to 2×10⁷ CFU/microliter. A total of 25 microliters adjusted inoculum (5×10⁵ bacteria) was added to each well. The samples were incubated for 2.5 hours at 37° C. To determine the bactericidal activity of the peptides, 40 microliters of a 1:100 dilution of the sample was plated onto a Brucella blood agar plate. The plate was incubated for 72 hours under anaerobic conditions at 37° C. The appearance of bacterial colonies was evaluated and documented. The MBC was determined as a 3 log or greater reduction in the number of P. acnes colony forming units per milliliter (CFU/milliliter) after a treatment with a peptide composition for a period of 2.5 hours compared to the negative control.

TABLE 4B
Minimum bactericidal concentration (MBC)
of Granulysin peptides for P. acnes

| | MBC |
|---|---|
| peptide | [micromolar units] |
| 30 | 8 |
| 32 | 2 |
| 64 | 8 |

Other microbial strains can be utilized in the assays described above, such as, P. aeruginosa, S. epidermidis, P. ovale, C. albicans, and T rubrum. P. aeruginosa is cultured in nutrient broth and agar, S. epidermidis is cultured in nutrient broth and agar; C. albicans is cultured in YM broth and agar; P. ovale is cultured in Emmons' modification of Sabouraud's agar and T. rubrum is cultured in Emmons' modification of Sabouraud's agar. Peptide compositions were tested for antimicrobial activity against P. aeruginosa, S. epidermidis and C. albicans in assays presented in Example 9.

### Example 3

### Human Sebum Assay for Determining Activity of Antimicrobial Peptides

Sebum was harvested from healthy human volunteers using Biore® deep cleansing pore strips. The seburn plugs were collected in a microtube with a tweezer. The seburn was pelleted by centrifugation at 14000 rpm for 1 minute. The seburn was resusupended in 1% TSB, 10 mM Na₂HPO₄ into fine particulate suspension. The assay was performed in a total volume of 50 microliters. Twenty-five microliters of seburn suspension were added to 25 microliters of assay buffer (20% reinforced Clostridium broth, 100 mM sodium carbonate) with or without test peptide composition. The samples were incubated for 150 minutes at 37° C. To determine the number of viable bacteria, a serial dilution was prepared (10 fold dilutions to 1/100000). A 40 microliter sample of each dilution was spotted onto a Brucella blood agar plate. After incubation for 72 hours bacterial growth was documented (Alpha Innotech documentation system). The number of CFU/milliliter was determined by counting bacteria colonies. The efficacy of a peptide is indicated by the reduction of CFU/milliliter compared to the untreated control.

TABLE 5
Bactericidal activity of peptide compositions for
indigenous bacteria in human sebum

| | Treatment (peptide composition from Table 3) | | |
|---|---|---|---|
| | | 30 | 64 |
| | none | (250 Micromolar) | (250 Micromolar) |
| cfu/ml | 8500000 | 1000 | 4100 |

### Example 4

### Hemolysis Assay for Determining Toxicity of Peptide Compositions

Hemoglobin-release assays with human red blood cells (RBC) were performed to determine the hemolytic activity of granulysin peptides. Human RBCs (San Diego Blood Bank, San Diego, Calif.) were washed three times in PBS buffer (8 mM Na₂HPO₄, 1.5 mM KH₂PO₄, 140 mM NaCl and 2.7 mM KCl, pH 7.4), resuspended in PBS to the concentration of 5% (v/v). The RBC were then exposed to different peptide compositions at various concentrations (100-6.25 micromolar) and incubated at 37° C. for 60 minutes in a total volume of 120 microliters. Every experiment included a negative control without peptide and maximum control treated with 5% TX-100 for complete hemoglobin release. Samples were centrifuged for 1 minute at 13,000 g to remove debris and intact cells. The hemoglobin-containing supernatant was removed and transferred to a microwell plate. Hemoglobin release was quantified at A540 using a Versamax plate reader (Molecular Devices). The degree of hemolysis is expressed as percentage of hemolysis compared to the untreated control, using the following formula:
% hemolysis=(sample−neg. control)/(max. control−neg. control)×100.

TABLE 6
Hemolytic effect of Granulysin peptides on human red
blood cells [in % of untreated control]

| | |
|---|---|
| concentration | Peptide composition in Table 3 |

| [Micromolar] | 30 | 32 | 64 | 85 |
|---|---|---|---|---|
| 100 | 0% | 2% | 81% | 83% |
| 50 | n.d. | n.d. | 46% | 53% |
| 25 | n.d. | n.d. | 29% | 36% |
| 12.5 | n.d. | n.d. | 15% | 26% |
| 6.25 | n.d. | n.d. | 14% | 16% |

### Example 5

### In Vitro Cytotoxicity Assay for Mammalian Cells

Toxicity of peptide compositions for mammalian cells was determined by using a metabolic viability assay. For this assay 4×10⁵ HeLa cells were seeded per well into a microwell plate (50 microliters volume). Jurkat cells also were utilized in separate experiments. The cells were incubated for 16 hours at 37° C. under mammalian cell culture conditions. Two fold serial dilutions for each peptide were prepared and added to the cells (final volume 100 microliters) and incubated for 16 hours at 37° C. For each dilution series control wells containing cells without peptide were included. The metabolic activity and viability of the cells were determined by using a commercially available assay (CellTiter96 assay, Promega Corp.). According to manufacturer's protocol 20 microliters of CellTiter reagent was added to each well and incubated at 37° C. for 1-4 hours. The CellTiter assay is a commercial version of the MTT assay, which measures the conversion of a tetrazolium peptide composition into a colored formazan salt in metabolically active cells. The formation of the formazan product can be measured at 470 nm and is generally accepted as a measure of cellular viability. The cells were incubated at 37° C. and monitored for color development. The conversion of CellTiter reagent was measured at 470 nm using a Versamax plate reader (Molecular Devices). The viability of peptide treated cells is expressed as IC50 (i.e., concentration that leads to a 50% reduction of viability relative to the untreated control). Table 7A reports IC50 values for HeLa cells cultured in OptiMEM or 10% FBS when contacted with peptide compositions from Table 3, and Table 7B reports IC50 values for HeLa cells or Jurkat cells contacted with peptide compositions from Table 3.

TABLE 7A

| | |
|---|---|
| Peptide | |
| Composition | IC50 (micromolar) |

| from Table 3 | OptiMEM | 10% FBS |
|---|---|---|
| 30 | >50 | >100 |
| 32 | 1.1 | 3-10 |
| 64 | 3-10 | 10-30 |
| 65 | 3-5 | 10-30 |
| 66 | 3-10 | 10-30 |

TABLE 7B

| | |
|---|---|
| Peptide | |
| Composition | IC50 (micromolar) |

| from Table 3 | Jurkat | HeLa |
|---|---|---|
| 1 | >100 | >100 |
| 30 | >100 | >100 |
| 33 | 67.5 | >100 |
| 76 | 3.3 | 5.49 |
| 90 | 5-15 | 5-15 |

### Example 6

### Assay for Detecting Anti-inflammation Activity of Peptide Compositions

An assay that detected reductions in IL-12 released in whole human blood cells (PBMCs) was utilized to detect reductions in inflammation elicited by peptide compositions described herein. The assay utilized the following materials and reagents: whole human blood cells; P. acnes; peptides 55, 67 and 93 from Table 3; IL-12p70 ELISA kit (eBiosciences); RPMI 1640 growth medium; PBS; and a 96-well microtiter plate. PBMCs were prepared from whole human blood cells according to standard procedures and cells were resuspended in RPMI 1640 and 10% FCS. Cells were seeded at 250,000 cells per well in a 96-well microtiter plate and incubated for 90 minutes. Unattached cells then were removed and cells adhering to the plate were washed three times with PBS. 100 microliters of medium was added to the cells in each well and the cells were incubated over night at 37° C. The next day P. acnes lysates were prepared by treating cells with 10 microliters of P. acnes in the presence of peptides 55, 67 and 93 from Table 3 at final concentrations of 12.5 micrograms per milliliter, 6.25 micrograms per milliliter, 3.1 micrograms per milliliter, 1.56 micrograms per milliliter and 0.78 micrograms per milliliter in a volume of 100 microliters per well. Controls included untreated cells, P. acnes alone or a peptide alone. The cells were incubated over night at 37° C., and cell supernatants were collected and stored at −80° C. for use in an IL-12 ELISA assay (eBiosciences). A 1:1 dilution of supernatant was utilized to perform the IL-12 ELISA following the protocol provided by the manufacturer.

Results of the assay demonstrated that peptides 67 and 93 from Table 3 showed a dose response of reduction of IL-12 release in PBMs stimulated by P. acnes. Peptides 93,76 and 55 were characterized in the assay as having an IC50 value of 0.8, 1.12 and greater than 20 micromolar, respectively.

### Example 7

### Mouse Skin Assay for Determining Activity of Peptide Compositions

P. acnes was grown in Clostridial broth under anaerobic conditions at 37° C. S. aureus was grown in tryptic soy broth (TSB) overnight. Prior to using the bacteria, the culture was subjected to centrifugation at 4000 rpm, washed with 2× carbonate buffer (100 mM NaCO₃; 2 mM MgCl₂) and then resuspended in Clostridial broth (for P. acnes) or 2× carbonate buffer with 10% TSB (for S. aureus). Balb/c mice were euthanized by an overdose of anesthetic, skinned and the skins swabbed with 70% EtOH to remove indigenous bacterial skin flora. Skin was placed between acrylic plates of a specialized multiwell plate (FIGS. 1A-1D). Ten microliters of bacteria was placed on the skin, incubated for 1 hour and at appropriate time points 500 micromolar to 1 micromolar peptide composition were added to the wells containing the bacteria. Bacteria were harvested with 100 microliters of Clostridial broth or TSB, were serially diluted and 20 microliters of the dilutions spotted onto blood agar or TSB plates to assess percent recovery and/or survival. Percent recovery was calculated by determining the ratio of recovered bacteria to an initial titer.

TABLE 8
Bactericidal peptide composition for P. acnes on mouse skin

| | P. acnes + peptide | |
|---|---|---|
| | composition | |
| Incubation time | 35 in Table 3 | P. acnes untreated |
| 120 min | 4.5 × 10⁴ cfu/ml | 0.9 × 10⁸ cfu/ml |

### Example 8

### Assay for Skin Penetration and Sebum Targeting of Peptide Compositions

To observe tissue penetration, peptide compositions are synthesized with a fluorogenic molecule, such as fluorescein, attached to the side chain of a C-terminal lysine residue. A stock solution of the peptide composition is prepared by dissolving it in water. A methylcellulose gel stock is prepared by dissolving methylcellulose powder in 1.8% sodium chloride solution (2x saline). The composition of the stock gel is usually 2% methylcellulose in 2× saline. Equal amounts of peptide composition stock and methylcellulose gel stock are mixed together to form a homogeneous gel containing 0.5 micromolar peptide in 1% gel. This gel is applied to the skin on the back of a mouse under anesthesia. One hour after the application of the peptide gel, excess peptide gel is removed by a wet lab tissue paper. The mouse is sacrificed. Skin samples are cut, frozen in freezing medium, such as OCT (Sakura Findtechnical, Japan), and sectioned into 10 micrometer thick sections with a cryostat. The sections are mounted on a glass slide and observed under a fluorescence microscope. The relative amount of fluorescence sometimes is quantified with a CCD camera with defined settings.

### Example 9

### Additional Microbial Inhibition Concentration (MIC) Assays for Determining Activity of Peptide Compositions

One method for determining the MIC for antimicrobial peptides in aerobic organisms (Staphylococcus aureus ATCC 29213, Salmonella dublin (Lane), Pseudomonas aeruginosa ATCC 27853, Escherichia coli ATCC 25922 and Staphylococcus epidermidis ATCC 12228) was performed according to the procedures from the National Committee for Clinical Laboratory Standards (NCCLS) document M7-A6. Cation-adjusted Mueller Hinton broth was used in the broth microdilution method. In brief, a log-phase bacterial suspension was used to inoculate wells of a 96-well plate so that the final inoculum for each well contained 5×10⁵ to 1×10⁶ CFU/milliliter. Each antimicrobial peptide was serially (2-fold) diluted for a final concentration range between 64 micrograms/milliliter and 1 micrograms/milliliter in a total volume of 100 microliters. Plates were incubated overnight at 37° C. The lowest concentration of peptide that contained no visible growth as evidenced by a lack of turbidity when compared to the control (no peptide) was determined as the MIC. Unless otherwise noted, MIC values listed in Table 9 utilized this method of MIC determination.

An additional method that was used to determine the MIC in aerobic organisms for selected antimicrobial peptides was a non-standard broth microdilution method that used an assay buffer composed of 20% tryptic soy broth (TSB), 50 mM Na₂CO₃ pH7.4 and 1 mM MgCl₂. In brief, a log-phase bacterial suspension was used to inoculate wells of a 96-well plate so that the final inoculum for each well contained 5×10⁵ to 1×10⁶ CFU/milliliter. Antimicrobial peptide was serially (2-fold) diluted for a final concentration range between 100 microgram/milliliter and 1.56 microgram/milliliter in a total volume of 100 milliliters. Plates were incubated overnight at 37° C. The lowest concentration of peptide that contained no visible growth as evidenced by a lack of turbidity when compared to the control (no peptide) was determined as the MIC. MIC values that utilized this method of MIC determination are indicated in Table 9 with a superscript letter “b.”

One method used for determining the MIC for antimicrobial peptides in anaerobic organisms (Propionibacterium acnes ATCC 6919) was the agar dilution method referenced in the NCCLS document M11-A6. Molten Brucella agar supplemented with laked sheep blood, hemin and vitamin K, was cooled to 50° C. and antimicrobial peptide was added for a final concentration range of 64 microgram/milliliter to 1 microgram/milliliter and poured into sterile petri dishes. A bacterial suspension was prepared to allow for a final inoculum on the plate of 1.5×10⁸ CFU/milliliter. After the suspension absorbed into the agar surface, the plates were incubated for 48-96 hours at 37° C. in a GasPak anaerobic chamber. The lowest concentration of peptide that contained no colony growth on the agar plate was determined as the MIC. MIC values that utilized this method of MIC determination are indicated in Table 9 with a superscript letter “c.”

A second method that was used to determine the MIC in P. acnes for selected antimicrobial agents was a non-standard broth microdilution method that used an assay buffer composed of reinforced Clostridial broth. Briefly, a log-phase bacterial culture of P. acnes was used to inoculate wells of a 96-well plate so that the final inoculum for each well contained 5×10⁵ to 1×10⁶ CFU/milliliter. Antimicrobial peptide was serially (2-fold) diluted for a final concentration range between 64 microgram/milliliter and 1 microgram/milliliter or 100 microgram/milliliter and 1.56 microgram/milliliter in a total volume of 100 microliters. Plates were incubated for 48-72 hours at 37° C. in a GasPak anaerobic chamber. The lowest concentration of peptide that contained no visible growth as evidenced by a lack of turbidity when compared to the control (no peptide) was determined as the MIC. MIC values that utilized this method of MIC determination are indicated in Table 9 with a subscript letter “d.”

The method for determining the MIC for antimicrobial peptides in yeasts (Candida albicans ATCC 18804) was performed according to the procedures in the NCCLS document M27-A2. Briefly, RPMI 1640 supplemented with glutamine and phenol red as a pH indicator was used in the broth microdilution method. A 96-well plate was inoculated so that the final inoculum of yeast for each well contained between 0.5×10³ and 2.5×10³ CFU/milliliter. Antimicrobial peptide was serially diluted (2-fold) for a final concentration range between 64 microgram/milliliter and 1 microgram/milliliter in a total volume of 100 microliters. Plates were incubated for 24 to 48 hours at 37° C. The lowest concentration of peptide that contained no visible growth was determined as the MIC. MIC values that utilized this method of MIC determination are indicated in Table 9 with a subscript letter “e.” “Peptide Number” corresponds to peptide compositions and corresponding designations in Table 3.

TABLE 9
MIC Values for Antimicrobial Peptides (ug/mL)

| PEPTIDE | | | | | | | |
|---|---|---|---|---|---|---|---|
| NUMBER | S. aureus | S. dublin | P. acnes | S. epidermidis | P. aeruginosa | E. coli | C. albicans |
| 3 | 6b | 6b | 6d | >64 | >64 | >64 | ND |
| 5 | >64 | >64 | ND | ND | ND | ND | ND |
| 6 | >64 | >64 | ND | ND | ND | ND | ND |
| 7 | >64 | >64 | ND | ND | ND | ND | ND |
| 8 | >64 | >64 | ND | ND | ND | ND | ND |
| 9 | >64 | >64 | ND | ND | ND | ND | ND |
| 10 | >64 | 64 | ND | ND | ND | ND | ND |
| 11 | 64 | 32 | ND | ND | ND | ND | ND |
| 12 | 16 | 32 | >64c | ND | ND | ND | >64e |
| 13 | 25b | 25b | 25d | ND | ND | ND | ND |
| 18 | >64 | >64 | ND | ND | ND | ND | ND |
| 19 | >64 | 32 | ND | ND | ND | ND | ND |
| 28 | >64 | >64 | ND | ND | ND | ND | ND |
| 31 | 3b | 6b | 12.5d | 8 | >64 | >64 | ND |
| 32 | 3b | 3b | 3d | ND | ND | ND | ND |
| 33 | >64 | 64 | 12.5d | 32 | >64 | >64 | 16e |
| 34 | >64 | >64 | 6d | ND | ND | ND | ND |
| 35 | >64 | >64 | 6d | ND | ND | ND | ND |
| 36 | 6b | 6b | 25d | ND | ND | ND | ND |
| 37 | 25b | 25b | 100d | >64 | >64 | >64 | 64e |
| 38 | >64 | >64 | 25d | ND | ND | ND | 16e |
| 56 | >64 | 8 | 6d | 2 | >64 | 4 | 64e |
| 61 | >64 | >64 | 12.5d | ND | ND | ND | ND |
| 63 | 8 | 16 | 12.5d | 2 | 64 | 32 | 16e |
| 64 | 8 | 16 | >64c | 1 | >64 | 64 | 32e |
| 65 | 3b | 6b | 25d | ND | ND | ND | ND |
| 66 | 25b | 25b | 100d | ND | ND | ND | ND |
| 67 | 64 | 64 | ND | ND | ND | ND | ND |
| 68 | 32 | 16 | ND | 8 | >64 | 64 | ND |
| 69 | 4 | 16 | 12.5d | ND | ND | ND | 16e |
| 70 | 8 | 32 | 64c | 1 | 8 | 4 | 8e |
| 76 | 2 | 16 | 64c | 1 | 32 | 4 | 16e |
| 77 | 16 | 16 | 12.5d | ND | ND | ND | 8e |
| 78 | 8 | 8 | 32c | 1 | 8 | 8 | 8e |
| 80 | 16 | 16 | >64c | 2 | >64 | 32 | ND |
| 81 | 64 | 32 | ND | 64 | >64 | 64 | ND |
| 84 | ND | ND | ND | 32 | >64 | >64 | ND |
| 85 | ND | ND | ND | 2 | >64 | 8 | ND |
| 86 | ND | ND | ND | 32 | >64 | >64 | ND |
| 87 | 4 | 8 | >64c | ND | ND | ND | 16e |
| 88 | 3b | 3b | 25d | ND | ND | ND | 32e |
| 89 | 4 | 4 | 25d | ND | ND | ND | 32e |
| 90 | 4 | 4 | 8c | 4 | 3b | 4 | 16e |
| 91 | >64 | >64 | 25d | ND | ND | ND | 16e |
| 92 | >64 | >64 | 25d | ND | ND | ND | 8e |
| 93 | >64 | >64 | ND | >64 | >64 | >64 | ND |
| 94 | >64 | >64 | ND | 16 | >64 | >64 | ND |
| 95 | 12.5b | 6b | 12.5d | ND | ND | ND | 16e |
| 96 | >64 | >64 | 12.5d | 12.5b | 50b | ND | 8e |
| 97 | 16 | 32 | 64c | 2 | 32 | 32 | 8e |
| 98 | 4 | 16 | >64c | 8 | 64 | 16 | 16e |
| 99 | 4 | >64 | >64c | 4 | >64 | 32 | 32e |
| 100 | 8 | >64 | >64c | 16 | >64 | >64 | 32e |
| 101 | 4 | >64 | >64c | 2 | 32 | 32 | >64e |
| 102 | 4 | >64 | >64c | 8 | 64 | >64 | 64e |
| 103 | >32 | >64 | ND | 64 | >64 | >64 | >64e |
| 104 | 4 | >64 | >64c | >64 | >64 | >64 | >64e |
| 105 | 16 | >32 | 32c | 8 | 32 | 32 | ND |
| 106 | 8 | >64 | >64c | 16 | >64 | >64 | ND |
| 107 | >64 | >64 | ND | ND | ND | ND | ND |
| 112 | >64 | >64 | ND | ND | ND | ND | >64e |
| 113 | 4 | 16 | >64c | 2 | >64 | 8 | ND |
| 114 | 4 | 4 | ND | ND | ND | ND | ND |
| 115 | 8 | 16 | 4c | 4 | 4 | 8 | 16e |
| 116 | 8 | 8 | 8c | 4 | 8 | 8 | 32e |
| 117 | 32 | >64 | ND | 16 | 64 | >64 | 16e |
| 118 | 8 | >64 | 16c | 8 | 32 | 64 | 32e |
| 119 | 32 | 64 | ND | 16 | 32 | 64 | 16e |
| 120 | 16 | >64 | 64c | 16 | 64 | >64 | 64e |
| 121 | >64 | >64 | ND | >64 | >64 | >64 | >64e |
| 122 | 8 | 32 | 32c | 8 | 16 | 64 | 32e |
| 123 | >64 | >64 | ND | 64 | >64 | >64 | 64e |
| 124 | >64 | >64 | ND | >64 | >64 | >64 | 64e |
| 125 | >64 | >64 | ND | >64 | >64 | >64 | >64e |
| 126 | >64 | >64 | ND | >64 | >64 | >64 | 8e |
| 128 | 16 | 32 | 16c | 8 | 32 | 32 | 32e |
| 129 | >64 | >64 | ND | 32 | >64 | >64 | >64e |
| 130 | 4 | 16 | 8c | 2 | 8 | 8 | 16e |
| 131 | 4 | 8 | 2c | 2 | 8 | 8 | 16e |
| 132 | 4 | 4 | 8c | 2 | 32 | 4 | 4e |
| 133 | 4 | 8 | 2c | 2 | 32 | 16 | 2e |
| 134 | 4 | 2 | 2c | 1 | 32 | 4 | 1e |
| 135 | 2 | 2 | ND | 1 | 4 | 4 | 2e |
| 136 | 4 | 8 | 8c | 1 | 32 | 8 | 1e |
| 138 | >64 | >64 | ND | >64 | >64 | >64 | 2e |
| 139 | 4 | 8 | 16c | 2 | 8 | 8 | 8e |
| 140 | 2 | 4 | 16c | 2 | 8 | 4 | 2e |
| 141 | 16 | 16 | 32c | 4 | >64 | 16 | 2e |
| 142 | 64 | >64 | ND | 8 | >64 | 64 | 4e |
| 143 | 4 | 4 | 2c | 2 | 16 | 4 | 4e |
| 144 | 32 | 64 | ND | 8 | >64 | >64 | 32e |
| 145 | >64 | >64 | ND | 16 | >64 | >64 | 2e |
| 146 | 64 | 64 | ND | 4 | >64 | >64 | 2e |
| 147 | 64 | 64 | ND | 2 | >64 | 64 | 2e |
| 148 | >64 | >64 | ND | >64 | >64 | >64 | 4e |
| 149 | 16 | 32 | 32c | 4 | >64 | 64 | ND |
| 150 | 8 | 32 | >64c | 4 | >64 | 64 | ND |
| 151 | 32 | 8 | ND | 4 | >64 | 64 | ND |
| 153 | 8 | 64 | ND | 8 | 64 | 32 | ND |
| 154 | 4 | 8 | ND | 8 | >64 | 64 | ND |
| 155 | 64 | 16 | ND | 16 | >64 | >64 | ND |
| 156 | 32 | 8 | ND | 16 | >64 | 64 | ND |
| 158 | 8 | 16 | 8c | 2 | 4 | 16 | 32e |
| 159 | 8 | 64 | ND | 8 | 32 | 16 | ND |
| 161 | 8 | >64 | ND | 4 | >64 | 16 | ND |
| 162 | 4 | 16 | ND | 8 | >64 | 32 | ND |
| 163 | 4 | 8 | >64c | 8 | >64 | 16 | ND |
| 168 | 16 | 16 | 64c | 1 | 32 | 2 | 32e |
| 169 | 4 | 8 | 64c | 1 | 4 | 2 | ND |
| 170 | 2 | 2 | ND | ND | ND | ND | ND |
| 171 | 8 | 8 | ND | ND | ND | ND | ND |
| 172 | >64 | >64 | ND | 64 | >64 | >64 | ND |
| 173 | 16 | 32 | >64c | 2 | 16 | 8 | 64e |
| 174 | 8 | 16 | 64c | 1 | 4 | 2 | 32e |
| 175 | 4 | 4 | 32c | 1 | 2 | 4 | 8e |
| 176 | 8 | 16 | 64c | 1 | 32 | 8 | 4e |
| 178 | >64 | 16 | ND | 2 | 16 | 8 | ND |
| 179 | 32 | 64 | ND | 8 | 64 | 32 | ND |
| 180 | 8 | 16 | 64c | 1 | 4 | 4 | 16e |
| 181 | 4 | 16 | 64c | 1 | 4 | 4 | 8e |
| 182 | 32 | 32 | ND | 4 | 64 | >64 | ND |
| 183 | >64 | >64 | ND | ND | ND | ND | ND |
| 184 | 64 | 16 | ND | 2 | >64 | 16 | ND |
| 185 | 4 | 16 | 32c | 1 | 16 | 8 | 32e |
| 186 | 4 | 16 | 16c | 1 | 4 | 4 | 16e |
| 188 | >64 | >64 | ND | 16 | >64 | 64 | ND |
| 189 | 32 | 8 | ND | 4 | 32 | 16 | ND |
| 190 | 8 | ND | 4 | 2 | 8 | 4 | ND |
| 191 | 64 | 8 | ND | 4 | >64 | 64 | ND |
| 192 | >64 | >64 | ND | ND | ND | ND | ND |
| 193 | 16 | 16 | 8c | 2 | 16 | 8 | 32e |
| 194 | 2 | 4 | 4c | 1 | 2 | 2 | 16e |
| 195 | 2 | 4 | 8c | 1 | 4 | 4 | 8e |
| 196 | 64 | 64 | ND | 32 | >64 | >64 | ND |
| 197 | >64 | >64 | ND | ND | ND | ND | ND |
| 198 | 16 | 32 | 16c | 4 | 64 | 16 | 64e |
| 199 | 2 | 8 | 8c | 1 | 4 | 4 | 32e |
| 200 | 2 | 8 | 8c | 1 | 4 | 4 | 8e |
| 201 | 16 | 16 | 16c | 2 | >64 | 32 | 2e |
| 202 | >64 | >64 | >64c | ND | ND | ND | ND |
| 203 | 32 | >64 | ND | ND | ND | ND | ND |
| 204 | 8 | 32 | >64c | 2 | 32 | 8 | ND |
| 206 | >64 | >64 | ND | 32 | >64 | 64 | ND |
| 207 | 16 | 64 | 16c | 4 | 32 | 32 | ND |
| 208 | 4 | 8 | 2c | >64 | >64 | >64 | ND |
| 209 | 16 | 32 | 32c | 8 | >64 | >64 | 8e |
| 210 | 4 | 4 | 8c | 2 | 16 | 8 | ND |
| 211 | 4 | 4 | 4c | 2 | 64 | 8 | ND |
| 212 | 4 | 4 | 2c | 2 | 64 | 8 | ND |
| 214 | 16 | 16 | >64c | 4 | >64 | 32 | 4e |
| 216 | 8 | 16 | ND | ND | ND | ND | ND |
| 217 | 1 | 2 | ND | 2 | 8 | 16 | ND |
| 218 | >64 | >64 | ND | ND | ND | ND | ND |
| 219 | 4 | 4 | 16c | 2 | 4 | 8 | ND |
| 220 | 2 | 2 | 16c | 1 | 8 | 4 | ND |
| 221 | 4 | 8 | 32c | 2 | 8 | 8 | 2e |
| 222 | 4 | >64 | >64c | 1 | 8 | 16 | 2e |
| 223 | 4 | 32 | >64c | 2 | 8 | 16 | 4e |
| 224 | 2 | 4 | 32c | 2 | 16 | 8 | ND |
| 225 | 1 | 1 | 8c | 2 | 16 | 16 | ND |
| 226 | 8 | 16 | 32c | 4 | >64 | 16 | ND |
| 227 | 32 | 32 | >64c | 32 | >64 | 64 | ND |
| 228 | 64 | 64 | ND | 16 | >64 | >64 | ND |
| 229 | 8 | 8 | ND | 4 | 8 | 8 | ND |
| 230 | >64 | >64 | ND | 4 | >64 | >64 | ND |
| 231 | 32 | >64 | ND | 4 | 32 | >64 | ND |
| 232 | >64 | >64 | ND | 16 | >64 | >64 | ND |
| 233 | 16 | >64 | 16c | 16 | 64 | 16 | ND |
| 234 | 64 | 64 | ND | 16 | >64 | >64 | ND |
| 235 | 16 | 32 | ND | 8 | 64 | 16 | ND |
| 236 | 32 | >64 | ND | 16 | >64 | 64 | ND |
| 237 | 4 | 4 | 16c | 1 | 8 | 4 | 16e |
| 238 | 8 | 8 | 32c | 8 | 64 | 8 | 16e |
| 239 | 16 | 64 | 8c | 4 | 32 | 16 | 64e |
| 240 | 2 | 4 | 16c | 2 | 16 | 4 | 16e |
| 241 | 4 | 16 | 64c | 8 | 32 | 32 | ND |
| 242 | 2 | 32 | 64c | 2 | 8 | 16 | ND |
| 243 | 4 | >64 | >64c | 2 | >64 | 32 | ND |
| 244 | 32 | >64 | ND | 64 | >64 | 64 | ND |
| 245 | 1 | 16 | 8c | 2 | 8 | 8 | ND |
| 246 | 4 | >64 | 32c | 2 | >64 | 32 | ND |
| 247 | 16 | 64 | >64c | 32 | >64 | 64 | 64e |
| 249 | 2 | 4 | 8c | 2 | 8 | 4 | 8e |
| 251 | >64 | >64 | ND | ND | ND | ND | 64e |
| 252 | 16 | 64 | 16c | 2 | 16 | 16 | 64e |
| 253 | 2 | 8 | 2c | 1 | 4 | 2 | 16e |
| 254 | 32 | >64 | ND | 4 | 64 | >64 | ND |

ND = Not Determined

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

1. A composition which comprises a peptide consisting of the amino acid sequence RSRWRDVARNFMR (SEQ ID NO: 283).

2. The composition of claim 1, wherein all of the amino acids in the peptide are L-isomer amino acids.

3. The composition of claim 1, wherein all of the amino acids in the peptide are D-isomer amino acids.

4. The composition of claim 1, wherein the peptide is a mixture of L-isomer and D-isomer amino acids.

5. The composition of claim 1, wherein the peptide is linked to a lipophilic molecule.

6. The composition of claim 5, wherein the lipophilic molecule has a log p value of +1 to +6.

7. The composition of claim 6, wherein the lipophilic molecule has a log p value of +3 to +4.5.

8. The composition of claim 5, wherein the lipophilic molecule is an acyl molecule.

9. The composition of claim 8, wherein the acyl molecule is a lauryl fatty acid molecule.

10. The composition of claim 8, wherein the acyl molecule is linked to the peptide by an amide linkage.

11. The composition of claim 8, wherein the acyl molecule is linked to the peptide at the N-terminus.

12. The composition of claim 8, wherein the acyl molecule is linked to the peptide at the C-terminus.

14. A pharmaceutical composition comprising a composition of claim 1 and a pharmaceutically acceptable carrier.

15. A method for reducing a microbe population, which comprises administering a composition comprising a peptide consisting of the amino acid sequence RSRWRDVARNFMR (SEQ ID NO: 283) in an amount that reduces the microbe population.

16. The method of claim 15, wherein the microbe is selected from the group consisting of Salmonella, Staphylococcus, Propionibacterium, Escherichia, Pseudomonas, Staphylococcus, Pityrosporum, Candida and Trichophyton.

17. The method of claim 16, wherein the microbe is selected from the group consisting of Salmonella dublin, Staphylococcus aureus, Propionibacterium acnes, Escherichia coli, Pseudomonas aeruginosa, Staphylococcus epidermidis, Pityrosporum ovale, Candida albicans and Trichophyton rubrum.

18. The method of claim 17, wherein the microbe is Propionibacterium acnes.

19. The method of claim 15, wherein the composition is administered to human skin.

20. The method of claim 19, wherein the composition is administered topically to the human skin.
